(19) United States
(12) Patent Application Publication
(10) Pub. No.: US 2019/0077774 A1
(43) Pub. Date: Mar. 14, 2019

(54) PEROXISOME PROLIFERATOR-ACTIVATED RECEPTOR AGONISTS

(71) Applicant: City of Hope, Duarte, CA (US)

(72) Inventors: Donna D. YU, Arcadia, CA (US); Barry M. Forman, Irvine, CA (US); Hongzhi Li, Arcadia, CA (US)

(21) Appl. No.: 16/039,001

(22) Filed: Jul. 18, 2018

Related U.S. Application Data
(60) Provisional application No. 62/539,072, filed on Jul. 31, 2017.

(51) Int. Cl. C07D 249/12; A61P 3/10; A61P 3/06; A61P 3/04; A61P 1/16

(52) U.S. Cl. CPC C07D 249/12; A61P 3/10; A61P 1/16; A61P 3/04; A61P 3/06

(57) ABSTRACT

Disclosed herein, inter alia, are compositions and methods useful for treating liver diseases and metabolic diseases.

### CROSS-REFERENCES TO RELATED APPLICATIONS

This application claims the benefit of U.S. Provisional Application No. 62/539,072, filed on Jul. 31, 2017, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-668001US Sequence Listing_ST25.txt, created Jul. 16, 2018, 1,091 bytes, machine format IBM-PC, MS Windows operating system, is hereby incorporated by reference.

### BACKGROUND

Non-alcoholic fatty liver disease (NAFLD) is a leading cause of chronic liver disease worldwide, especially owing to its close relationship with metabolic features, such as type 2 diabetes mellitus (T2DM), dyslipidemia and obesity. NAFLD presents a clinical spectrum, ranging from simple steatosis to its progressive form, known as non-alcoholic steatohepatitis (NASH), caused by an elevation in oxidative stress, which might further lead to hepatic fibrogenesis. NASH is expected to become the leading cause of liver transplants by 2020, and no drugs have been approved to treat it. The growing incidence of metabolic disease has led to an intense interest in identifying new molecular targets and pharmacologic agents to treat and/or prevent these metabolic disorders.

Type 2 diabetes in particular has become much more common, along with the obesity epidemic. Notably, obesity and type-2 diabetes are key risk factors associated with abnormal regulation of hepatic glucose production leading to Nonalcoholic steatohepatitis (NASH), an extreme form of nonalcoholic fatty liver disease (NAFLD), defined as the presence of hepatic steatosis with inflammation and hepatocyte injury. NASH is thus becoming a major health issue in close association with the worldwide epidemic of obesity and diabetes.

Numerous approaches have been examined for the potential treatment of NAFLD/NASH. Despite this massive effort, there is no drugs have been approved by the FDA for treatment of NASH. Thus, there is a critical need for new therapies that target earlier stages of NASH, and can be used as a prophylactic measure. Much attention has been paid recently to the functions of nuclear receptors PPARs for their potential roles as therapeutic targets implicated in the etiology of metabolic disorders.

Peroxisome proliferator-activated receptors (PPARs) are ligand-activated transcription factors in the nuclear hormone receptor superfamily for fatty acids that regulates glucose and lipid homeostasis.¹ Three subtypes of PPARs, namely, PPARα, PPARγ, and PPARδ, have been identified in various species, including humans. Each PPAR subtype seems to be expressed in its own tissue-specific manner and plays a pivotal role in lipid and lipoprotein homeostasis. Among the three isotypes, PPARα and PPARγ have been the most widely studied, as they have important roles in regulating glucose, lipids and cholesterol metabolism as well as in the fatty acid β-oxidation and homeostasis. PPARα was the first isoform to be identified as a regulator of gene expression in lipid metabolism. Its mechanism of action remained unknown until the discovery that fibrates activate and bind to PPARα.² PPARα has received much attention recently because it has important roles in regulation of metabolic homeostasis, inflammation, as well as the growth and differentiation of cells. Mice lacking PPARα have inhibition of cellular fatty acid turnover, resulting in massive hepatic and cardiac lipid accumulation. The metabolic abnormalities are due to altered expression of a range of metabolic enzymes that play a role for PPARα in lipid homeostasis.

In studies aimed at identifying the role of PPARα in the liver and related metabolic diseases, a variety of labs recently found that PPARα plays a key role in the regulation of lipid metabolism in human liver. PPARα mRNA and protein is highly expressed in normal mouse and human liver but was negatively correlated with the presence of NASH severity, visceral adiposity and insulin resistance and positively with adiponectin.³ Histological improvement is associated with an increase in expression of PPARα and its target genes. These findings suggest that loss of PPARα expression may contribute to the development of NASH and PPARα is a potential therapeutic target in NAFLD/NASH.⁴ Disclosed herein, inter alia, are solutions to these and other problems in the art.

### SUMMARY

In an aspect is provided a compound having the formula:

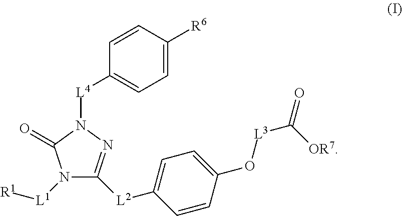

L¹ is a bond, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is unsubstituted C₁-C₆ alkylene.

L³ is substituted or unsubstituted C₁-C₆ alkylene.

L⁴ is unsubstituted C₁-C₄ alkylene.

R¹ is substituted or unsubstituted cycloalkyl, substituted or unsubstituted heterocycloalkyl, substituted or unsubstituted aryl, or substituted or unsubstituted heteroaryl.

R⁶ is unsubstituted C₁-C₄ alkyl.

R⁷ is hydrogen or, together with the oxygen to which it is attached, forms a prodrug moiety.

In an aspect is provided a pharmaceutical composition including a compound described herein, or pharmaceutically acceptable salt thereof, and a pharmaceutically acceptable excipient.

In an aspect is provided a method of treating non-alcoholic fatty liver disease (NAFLD), the method including administering to a subject in need thereof an effective amount of a compound described herein.

In an aspect is provided a method of treating non-alcoholic steatohepatitis (NASH), the method including administering to a subject in need thereof an effective amount of a compound described herein.

In an aspect is provided a method of treating a disease associated with peroxisome proliferator-activated receptor activity including administering to a subject in need thereof an effective amount of a compound described herein.

In an aspect is provided a method of treating obesity, the method including administering to a subject in need thereof an effective amount of a compound described herein.

In an aspect is provided a method of treating a hormone disorder, the method including administering to a subject in need thereof an effective amount of a compound described herein.

In an aspect is provided a method of treating dyslipidemia, the method including administering to a subject in need thereof an effective amount of a compound described herein.

In an aspect is provided a method of treating a lipid disorder, the method including administering to a subject in need thereof an effective amount of a compound described herein.

In an aspect is provided a method of treating a metabolic disorder, the method including administering to a subject in need thereof an effective amount of a compound described herein.

In an aspect is provided a method of treating syndrome X (metabolic syndrome), the method including administering to a subject in need thereof an effective amount of a compound described herein.

In an aspect is provided a method of treating diabetes, the method including administering to a subject in need thereof an effective amount of a compound described herein.

In an aspect is provided a method of treating type 1 diabetes, the method including administering to a subject in need thereof an effective amount of a compound described herein.

In an aspect is provided a method of treating type 2 diabetes, the method including administering to a subject in need thereof an effective amount of a compound described herein.

In an aspect is provided a method of treating fibrosis (e.g., liver fibrosis), the method including administering to a subject in need thereof an effective amount of a compound described herein.

In an aspect is provided a method of increase peroxisome proliferator-activated receptor activity, the method including contacting the peroxisome proliferator-activated receptor with a compound described herein.

In an aspect is provided a method of modulating the level of a lipid in a subject, the method including administering to a subject in need thereof an effective amount of a compound described herein.

### BRIEF DESCRIPTION OF THE DRAWINGS

FIG. 1. A focused small chemical library and chemical structures of DY121.

FIG. 2. Representative PPARα disclosed synthetic agonists.

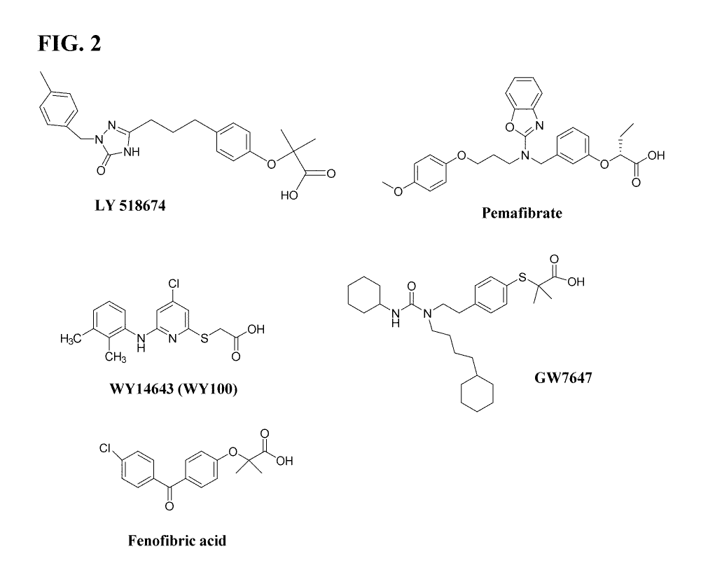

FIG. 3 Chemical structures of PPARα selective modulators DY121-132.

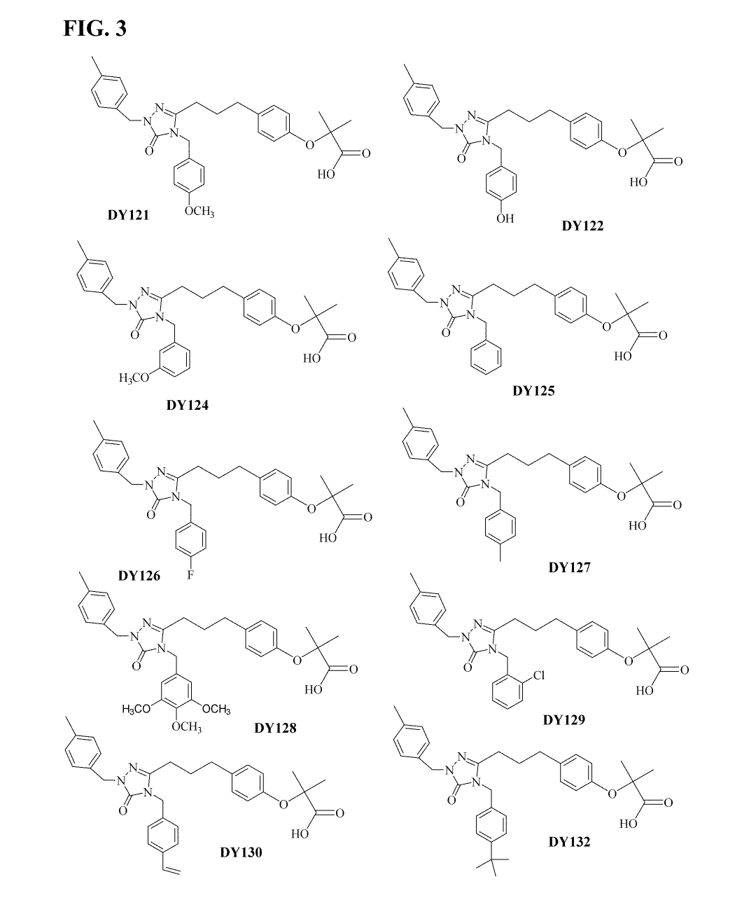

FIG. 4. Transfections: Mammalian transient transfections demonstrated activation of a reporter on a PPAR response element. Differences in fold activation suggest selective modulation of the PPARα receptor.

FIG. 5. Ligand Screening Results: DY series compounds and controls administered once to C57Bl/6J female mice at 8 weeks of age. Mice were treated at 3 mg/kg for all compounds except WY14643 (100 mg/kg). Compounds gavaged at 0900; mice sacrificed 24 h later. Ordered by degree of effect on triglyceride lowering. According to our screening test, DY series compounds reduce triglycerides.

FIG. 6. Liver gene expression of PPARα targets in mice treated at 3 mg/kg (WY100=WY14643 at 100 mg/kg) with PPARα agonists for 24 h. Data in same order as triglyceride suppression (FIG. 5). apo CIII and LPL expression inversely correlate with triglyceride suppression for many, but not all, compounds. ACOX1 and Cyp4A3 expression correlate with triglyceride suppression for many, but not all, compounds. Taken together, these data support the potential for selective modulation of PPARα in response to each compound.

FIG. 7. Dose-Response: Triglycerides: Dose-response suppression of triglycerides by several DY compounds, DY121, DY128, and DY129.

FIG. 8. Time Course: Triglycerides: Chronic effects of treatment with PPARα ligands (24 h vs 72 h). a) Body weight was unaffected by treatment with DY121, suggesting effects on triglycerides and gene expression are not due to toxicity. b) Suppression of triglycerides is maintained over 72 h in wild type but not PPARα-knockout mice. DY121 has significant effect on triglycerides:

FIG. 9. Chronic Effects—Weight: Time course of treatment over 7 days with several compounds. Only WY14643 caused acute weight loss, suggesting it may have some slight toxicity acutely, or mediates weight loss by suppressing appetite. No compounds suppressed weight at 7 days treatment.

FIG. 10. Chronic Effects—Lipids: Only WY14643 and DY121 suppressed triglycerides over the entire 7 day period tested. This effect appears to be mediated by enhanced disposition of free fatty acids with DY121 and possibly with WY14643.

FIG. 11. Chronic Effects—Lipid Transport: DY121 significantly suppressed expression of apoCIII at each time point; WY14643 suppressed apoCIII only after 7 days treatment. Only DY121 enhanced expression of LPL, suggesting that triglyceride lowering is through enhanced degradation of triglycerides and uptake of free fatty acids by peripheral tissues. DY121 significantly suppressed expression of apoCIII at each time point;

FIG. 12. Chronic Effects—Lipid Oxidation: ACOX1 expression was enhanced by PPARα activation, most consistently by DY121. At least some of the disappearance of FFA into peripheral tissues without concurrent weight gain may be due to microsomal oxidation.

FIG. 13. Chronic Effects—Lipid Oxidation: Cyp4A3 expression was enhanced by PPARα activation, most greatly by WY14643 but also consistently by GW7647 and DY121. Some of the FFA disappearance may be explained by peroxisomal oxidation.

FIG. 14. Triglycerides were suppressed roughly to the same extent by all treatments. Effects may be due to inhibition of synthesis or increased clearance from plasma; FFA, glycerol time course was similar between PBS, WY100 and GW3.FFA and glycerol time courses for DY121 showed early increase (lipolytic burst) followed by decline (suppressed lipolysis; enhanced uptake, enhanced FFA oxidation).

FIG. 15. Triglycerides were suppressed roughly to the same extent by all treatments. Unclear whether this is inhibition of synthesis or increased clearance from plasma FFA, glycerol time course was similar between PBS, WY100 and GW3. FFA and glycerol time courses for DY121 showed early increase (lipolytic burst) followed by decline (suppressed lipolysis; enhanced uptake, enhanced FFA oxidation).

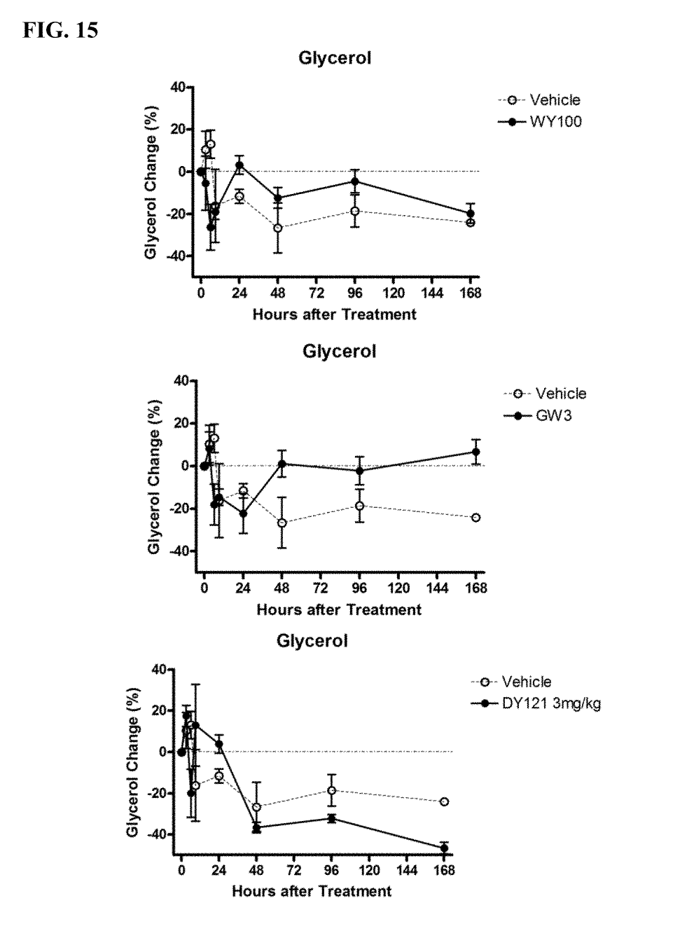

FIG. 16. Enhanced PPARα expression and/or activity (particularly in concert with PGC-1α) is expected to increase blood glucose by upregulating gluconeogenic enzymes like PEPCK. Glucose increased after 4 days with WY and GW, but not with DY121. This suggests selective modulation that may make DY121 an attractive therapy for diabetics with hypertriglyceridemia.

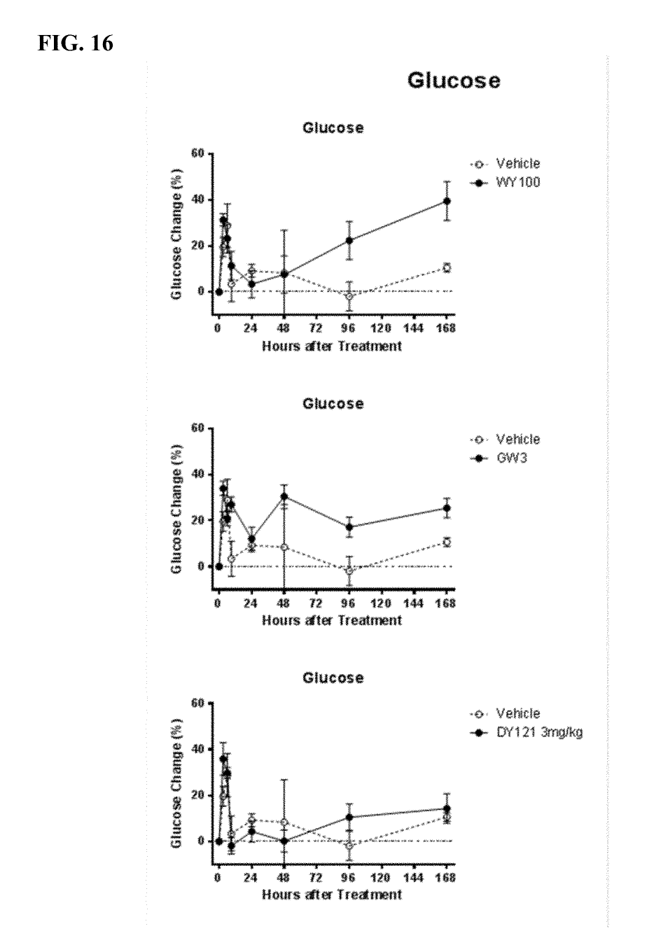

FIG. 17. Liver CPT-Ia is upregulated by chronic (>3 d) treatment with GW or DY121 but not WY.

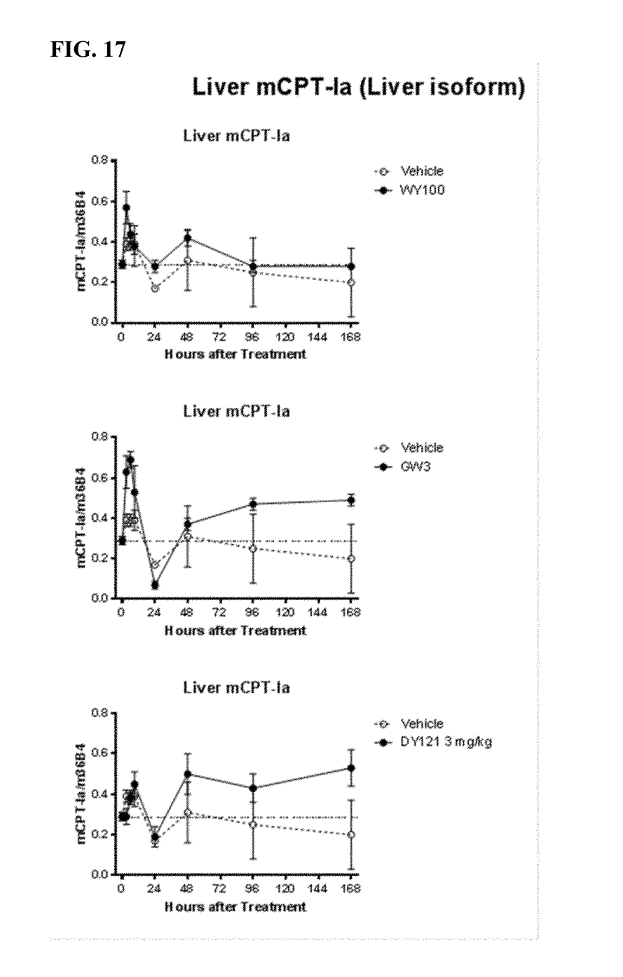

FIG. 18. While each compound upregulates ACADM, the temporal course is strikingly different for WY compared to GW and DY121, suggesting selective modulation of this PPARα target.

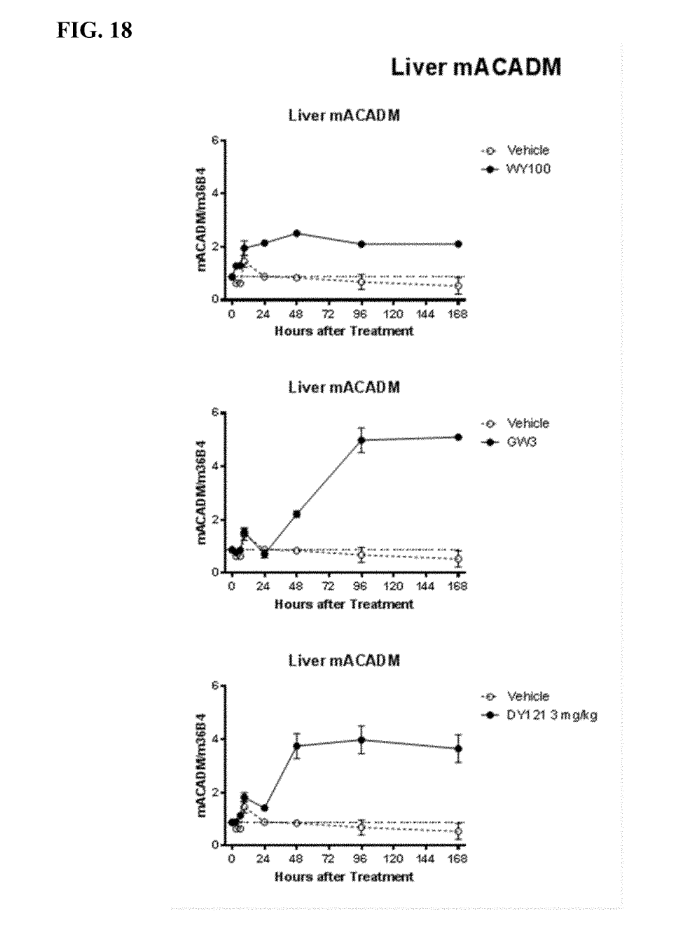

FIG. 19. Molecular modeling structure of PPARα LBD-DY121 complexes. The binding pose of DY121 (light grey colored) for 3 major interaction regions (R1 region, R4 region, and —COOH region) overlaps with the ligand GW409544 (dark grey colored) from X-ray structure in PDB 1k71.

FIG. 20. Interactions between PPARα LBD and DY121 molecule. The carboxyl group of DY121 forms hydrogen bonds (black dots) with S280, Y314 and Y464. The aromatic ring R3 forms π-stacking interaction with F318 and H440. The aromatic ring R1 region interacts with F273 and F351 via π-stacking network. The aromatic ring R4 region forms hydrophobic interactions with I272, V332, I339, and L344 residues.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbon atoms (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, S, B, As, or Si), and wherein the nitrogen and sulfur atoms may optionally be oxidized, and the nitrogen heteroatom may optionally be quaternized. The heteroatom(s) (e.g., O, N, P, S, B, As, or Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, P, S, B, As, or Si). A heteroalkyl moiety may include two optionally different heteroatoms (e.g., O, N, P, S, B, As, or Si). A heteroalkyl moiety may include three optionally different heteroatoms (e.g., O, N, P, S, B, As, or Si). A heteroalkyl moiety may include four optionally different heteroatoms (e.g., O, N, P, S, B, As, or Si). A heteroalkyl moiety may include five optionally different heteroatoms (e.g., O, N, P, S, B, As, or Si). A heteroalkyl moiety may include up to 8 optionally different heteroatoms (e.g., O, N, P, S, B, As, or Si).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

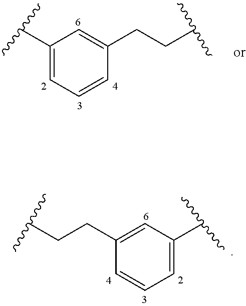 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cyclalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OC H₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCH F₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
   (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —O CHF₂, —OCH₂Cl, —OCH₂Br, —OCH₂F, —OCH₂I, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts. Non-limiting examples of salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent. A prodrug moiety is a moiety bonded to a compound to form a prodrug of the compound. A prodrug moiety may be removed from a prodrug (e.g., by enzymatic reaction such as by an esterase or amidase) to produce the active compound and resulting product prodrug moiety compound (e.g., by addition of a new hydrogen on the prodrug moiety in place of the original bond to the compound and/or hydrogen on the active compound in place of the original bond to the prodrug moiety). A prodrug may be converted to an active compound (e.g., compound not bound to the prodrug moiety) by a carboxylesterase in the liver. Esterases are found in a number of tissues, particularly the liver, kidney, and plasma. A prodrug may overcome pharmacokinetics problems of a parent compound. The parent form of a carboxylic acid containing compound described herein could be masked by binding a moiety (e.g., R⁷) to the oxygen of the carboxylic acid, resulting in a prodrug having an ester bond. A prodrug may provide reduced toxicity, improved therapeutic index, slow release of the parent drug and/or improved selectivity towards the target tissue or cell.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid residue in a protein “corresponds” to a given residue when it occupies the same essential structural position within the protein as the given residue. For example, a selected residue in a selected protein corresponds to S280 of human peroxisome proliferator-activated receptor alpha when the selected residue occupies the same essential spatial or other structural relationship as S280 in human peroxisome proliferator-activated receptor alpha. In some embodiments, where a selected protein is aligned for maximum homology with the human peroxisome proliferator-activated receptor alpha, the position in the aligned selected protein aligning with S280 is said to correspond to S280. Instead of a primary sequence alignment, a three dimensional structural alignment can also be used, e.g., where the structure of the selected protein is aligned for maximum correspondence with the human peroxisome proliferator-activated receptor alpha protein and the overall structures compared. In this case, an amino acid that occupies the same essential position as S280 in the structural model is said to correspond to the S280 residue.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein refers to conversion of a protein into a biologically active derivative from an initial inactive or deactivated state. The terms reference activation, or activating, sensitizing, or upregulating signal transduction or enzymatic activity or the amount of a protein decreased in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “peroxisome proliferator-activated receptor alpha” and “peroxisome proliferator-activated receptor α” refer to a protein (including homologs, isoforms, and functional fragments thereof) with peroxisome modulator activity. The term includes any recombinant or naturally-occurring form of peroxisome proliferator-activated receptor alpha or variants thereof that maintain peroxisome proliferator-activated receptor alpha activity (e.g. within at least 30%, 40%, 50%, 60%, 70%, 80%, 90%, 95%, or 100% activity compared to wildtype peroxisome proliferator-activated receptor alpha). In embodiments, the peroxisome proliferator-activated receptor alpha protein encoded by the PPARA or NR1C1 gene has the amino acid sequence set forth in or corresponding to Entrez 5465, UniProt Q07869, or RefSeq (protein) NP_005027. In embodiments, the peroxisome proliferator-activated receptor alpha gene has the nucleic acid sequence set forth in RefSeq (mRNA) NM_005036. In embodiments, the amino acid sequence or nucleic acid sequence is the sequence known at the time of filing of the present application. In embodiments, the sequence corresponds to NP_005027.2. In embodiments, the sequence corresponds to NM_005036.4. In embodiments, the peroxisome proliferator-activated receptor alpha is a human peroxisome proliferator-activated receptor alpha.

The terms “disease” or “condition” refer to a state of being or health status of a patient or subject capable of being treated with the compounds or methods provided herein. The disease may be a cancer. The disease may be NAFLD. The disease may be NASH. The disease may be a metabolic disease. The disease may be diabetes. The disease may be Obesity. The disease may be metabolic syndrome.

The terms “non-alcoholic fatty liver disease” or “NAFLD” refers to a liver disease characterized by fat deposits in the liver, which are not primarily due to excessive alcohol use. In embodiments, NAFLD may be related to or associated with insulin resistance, metabolic syndrome, diabetes mellitus type 2, obesity, hyperlipidemia, and/or high blood pressure.

The terms “non-alcoholic steatohepatitis” or “NASH” refers to a severe form of NAFLD characterized by fat deposits in the liver, which are not primarily due to excessive alcohol use. In embodiments, NASH may be related to or associated with insulin resistance, metabolic syndrome, diabetes mellitus type 2, obesity, hyperlipidemia, and/or high blood pressure. In embodiments, NASH is characterized by swelling of the liver, liver damage, liver scarring, or liver cirrhosis. In embodiments, NASH is associated with insulin resistance. In embodiments, NASH is characterized by liver inflammation, cell death, and/or fibrosis.

The terms “metabolic disease” refers to a disorder characterized by one or more abnormal metabolic processes in a subject. In embodiments, a metabolic disorder may be associated with, related to, or may be NAFLD, NASH, diabetes, insulin resistance, metabolic syndrome, diabetes mellitus type 2, obesity, hyperlipidemia, hyperglycemia, high serum triglycerides, and/or high blood pressure.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease means that the disease (e.g. NAFLD, NASH, obesity, diabetes) is caused by (in whole or in part), or a symptom of the disease is caused by (in whole or in part) the substance or substance activity or function. For example, a NASH associated with peroxisome proliferator-activated receptor (e.g., PPAR α) activity or function may be a NASH that results (entirely or partially) from aberrant peroxisome proliferator-activated receptor (e.g., PPAR α) function (e.g. enzyme activity, protein-protein interaction, signaling pathway) or a NASH wherein a particular symptom of the disease is caused (entirely or partially) by aberrant peroxisome proliferator-activated receptor (e.g., PPAR α) activity or function. As used herein, what is described as being associated with a disease, if a causative agent, could be a target for treatment of the disease. For example, a NASH associated with peroxisome proliferator-activated receptor (e.g., PPAR α) activity or function or a peroxisome proliferator-activated receptor (e.g., PPAR α) associated NAFLD, may be treated with a peroxisome proliferator-activated receptor (e.g., PPAR α) modulator or peroxisome proliferator-activated receptor (e.g., PPAR α) agonist.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propogated to other signaling pathway components. For example, binding of a peroxisome proliferator-activated receptor (e.g., PPAR α) protein with a compound as described herein may increase the interactions between the peroxisome proliferator-activated receptor (e.g., PPAR α) protein and downstream effectors or signaling pathway components, resulting in changes in cell growth, proliferation, survival, metabolism, secretion, or expression.

### II. Compounds

In an aspect is provided a compound having the formula:

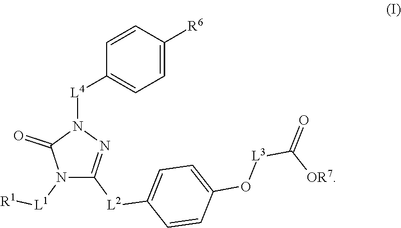

L¹ is a bond, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is unsubstituted C₁-C₆ alkylene.

L³ is substituted or unsubstituted C₁-C₆ alkylene.

L⁴ is unsubstituted C₁-C₄ alkylene.

R¹ is substituted or unsubstituted cycloalkyl, substituted or unsubstituted heterocycloalkyl, substituted or unsubstituted aryl, or substituted or unsubstituted heteroaryl.

R⁶ is unsubstituted C₁-C₄ alkyl.

R⁷ is hydrogen, oxo, halogen, —CX⁷₃, —CHX⁷₂, —CH₂X⁷, —OCX⁷₃, —OCH₂X⁷, —OCHX⁷₂, —CN, —SOₙ₇R^{7A}, —SOᵥ₇NR^{7A}R^{7B}, —NR^{7C}C(O)NR^{7A}R^{7B}, —N(O)ₘ₇, —NR^{7A}R^{7B}, —NR^{7C} NR^{7A}R^{7B}, —C(O)R^{7A}, —C(O)OR^{7A}, —C(O)NR^{7A}R^{7B}, —C(O)NR^{7A}R^{7B}, —OR^{7A}, —NR^{7A}SO₂R^{7B}, —NR^{7A}C(O)R^{7B}, —NR^{7A}C(O)OR^{7B}, —NR^{7A}OR^{7B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or, together with the oxygen to which it is attached, forms a prodrug moiety.

R^{7A}, R^{7B}, and R^{7C} are independently hydrogen, —CX₃, —CHX₂, —CH₂X, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl. X and X⁷ are independently —F, —Cl, —Br, or —I. m7 is 1 or 2. v7 is 1 or 2. n7 is an integer from 0 to 4.

In embodiments, L¹ is a bond or substituted or unsubstituted alkylene. In embodiments, L¹ is a bond or unsubstituted C₁-C₃ alkylene. In embodiments, L¹ is an unsubstituted C₁-C₃ alkylene. In embodiments, L¹ is an unsubstituted methylene. In embodiments, L¹ is a bond.

In embodiments, L² is unsubstituted C₂-C₄ alkylene. In embodiments, L² is unsubstituted n-propylene.

In embodiments, L³ is substituted or unsubstituted C₁-C₃ alkylene. In embodiments, L³ is substituted or unsubstituted C₁-C₃ alkylene. In embodiments, L³ is unsubstituted C₁-C₃ alkylene. In embodiments, L³ is unsubstituted C₁-C₃ alkylene. In embodiments, L³ is —C(CH₃)₂—.

In embodiments, L⁴ is unsubstituted C₁-C₃ alkylene. In embodiments, L⁴ is unsubstituted C₁-C₂ alkylene. In embodiments, L⁴ is —CH₂—.

In embodiments, R¹ is independently substituted or unsubstituted cycloalkyl (e.g., C₃-C₈, C₃-C₆, C₄-C₆, or C₅-C₆). In embodiments, R¹ is independently substituted cycloalkyl (e.g., C₃-C₈, C₃-C₆, C₄-C₆, or C₅-C₆). In embodiments, R¹ is independently unsubstituted cycloalkyl (e.g., C₃-C₈, C₃-C₆, C₄-C₆, or C₅-C₆). In embodiments, R¹ is independently substituted or unsubstituted heterocycloalkyl (e.g., 3 to 8 membered, 3 to 6 membered, 4 to 6 membered, 4 to 5 membered, or 5 to 6 membered). In embodiments, R¹ is independently substituted heterocycloalkyl (e.g., 3 to 8 membered, 3 to 6 membered, 4 to 6 membered, 4 to 5 membered, or 5 to 6 membered). In embodiments, R¹ is independently unsubstituted heterocycloalkyl (e.g., 3 to 8 membered, 3 to 6 membered, 4 to 6 membered, 4 to 5 membered, or 5 to 6 membered). In embodiments, R¹ is independently substituted or unsubstituted aryl (e.g., C₆-C₁₂, C₆-C₁₀, or phenyl). In embodiments, R¹ is independently substituted aryl (e.g., C₆-C₁₂, C₆-C₁₀, or phenyl). In embodiments, R¹ is independently unsubstituted aryl (e.g., C₆-C₁₂, C₆-C₁₀, or phenyl). In embodiments, R¹ is independently substituted or unsubstituted heteroaryl (e.g., 5 to 12 membered, 5 to 10 membered, 5 to 9 membered, or 5 to 6 membered). In embodiments, R¹ is independently substituted heteroaryl (e.g., 5 to 12 membered, 5 to 10 membered, 5 to 9 membered, or 5 to 6 membered). In embodiments, R¹ is independently unsubstituted heteroaryl (e.g., 5 to 12 membered, 5 to 10 membered, 5 to 9 membered, or 5 to 6 membered).

In embodiments, R¹ is independently R²-substituted or unsubstituted cycloalkyl (e.g., C₃-C₈, C₃-C₆, C₄-C₆, or C₅-C₆), R²-substituted or unsubstituted heterocycloalkyl (e.g., 3 to 8 membered, 3 to 6 membered, 4 to 6 membered, 4 to 5 membered, or 5 to 6 membered), R²-substituted or unsubstituted aryl (e.g., C₆-C₁₂, C₆-C₁₀, or phenyl), or R²-substituted or unsubstituted heteroaryl (e.g., 5 to 12 membered, 5 to 10 membered, 5 to 9 membered, or 5 to 6 membered). In embodiments, R¹ is independently unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹ is substituted or unsubstituted C₄-C₆ cycloalkyl, substituted or unsubstituted 4 to 6 membered heterocycloalkyl, substituted or unsubstituted phenyl, or substituted or unsubstituted 5 to 6 membered heteroaryl. In embodiments, R¹ is substituted or unsubstituted phenyl or substituted or unsubstituted 5 to 6 membered heteroaryl. In embodiments, R¹ is substituted or unsubstituted phenyl. In embodiments, R¹ is R²-substituted or unsubstituted phenyl or R²-substituted or unsubstituted 5 to 6 membered heteroaryl. In embodiments, R¹ is R²-substituted phenyl. In embodiments, R¹ is unsubstituted phenyl. In embodiments, R¹ is R²-substituted pyridyl. In embodiments, R¹ is unsubstituted pyridyl.

R² is independently oxo, halogen, —CX²³, —CHX²², —CH₂X², —OCX²³, —OCH₂X², —OCHX²², —CN, —SOₙ₂R^{2A}, —SOᵥ₂NR^{2A}R^{2B}, —NR^{2C}C(O)NR^{2A}R^{2B}, —N(O)ₘ₂, —NR^{2A}R^{2B}, —NR² CNR^{2A}R^{2B}, —C(O)R^{2A}, —C(O)OR^{2A}, —C(O)NR^{2A}R^{2B}, —C(O)NR^{2C}NR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is independently halogen, —CX²³, —CHX²₂, —CH₂X², —OCX²₃, —OCH₂X², —OCHX²₂, —CN, —SOₙ₂R^{2A}, —SOᵥ₂NR^{2A}R^{2B}, —NR^{2C}C(O)NR^{2A}R^{2B}, —N(O)ₘ₂, —NR^{2A}R^{2B}, —NR² CNR^{2A}R^{2B}, —C(O)R^{2A}, —C(O)OR^{2A}, —C(O)NR^{2A}R^{2B}, —C(O)NR²CNR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, two R² substituents (e.g., directly connected to adjacent atoms of R¹) may be joined to form a substituted or unsubstituted cycloalkyl, substituted or unsubstituted heterocycloalkyl, substituted or unsubstituted aryl, or substituted or unsubstituted heteroaryl.

R^{2A}, R^{2B}, and R^{2C} are independently hydrogen, —CX₃, —CHX₂, —CH₂X, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

m2 is independently 1 or 2.

v2 is independently 1 or 2.

n2 is independently an integer from 0 to 4.

X and X² are independently —Cl, —Br, —I or —F.

In embodiments, R² is independently oxo. In embodiments, R² is independently halogen. In embodiments, R² is independently —CX²₃. In embodiments, R² is independently —CHX²₂. In embodiments, R² is independently —CH₂X². In embodiments, R² is independently —OCX²₃. In embodiments, R² is independently —OCH₂X². In embodiments, R² is independently —OCHX²₂. In embodiments, R² is independently —CN. In embodiments, R² is independently —SOₙ₂R^{2A}. In embodiments, R² is independently —SOᵥ₂NR^{2A}R^{2B}. In embodiments, R² is independently —NR^{2C}C(O)NR^{2A}R^{2B}. In embodiments, R² is independently —N(O)ₘ₂. In embodiments, R² is independently —NR^{2A}R^{2B}. In embodiments, R² is independently —NR^{2C}NR^{2A}R^{2B}In embodiments, R² is independently —C(O)R^{2A}. In embodiments, R² is independently —C(O)OR^{2A}. In embodiments, R² is independently —C(O)NR^{2A}R^{2B}. In embodiments, R² is independently —C(O)NR^{2C}NR^{2A}R^{2B}. In embodiments, R² is independently —OR^{2A}. In embodiments, R² is independently —NR^{2A}SO₂R^{2B}. In embodiments, R² is independently —NR^{2A}C(O)R^{2B}. In embodiments, R² is independently —NR^{2A}C(O)OR^{2B}. In embodiments, R² is independently —NR^{2A}OR^{2B}. In embodiments, R² is independently —N₃. In embodiments, R² is independently —OH. In embodiments, R² is independently —NH₂. In embodiments, R² is independently —COOH. In embodiments, R² is independently —CONH₂. In embodiments, R² is independently —NO₂. In embodiments, R² is independently —SH. In embodiments, R² is independently halogen. In embodiments, R² is independently —F. In embodiments, R² is independently —Cl. In embodiments, R² is independently —Br. In embodiments, R² is independently —I. In embodiments, R² is independently —CF₃. In embodiments, R² is independently —CHF₂. In embodiments, R² is independently —CH₂F. In embodiments, R² is independently —OCF₃. In embodiments, R² is independently —OCH₂F. In embodiments, R² is independently —OCHF₂. In embodiments, R² is independently —OCH₃. In embodiments, R² is independently —OCH₂CH₃. In embodiments, R² is independently —OCH₂CH₂CH₃. In embodiments, R² is independently —OCH(CH₃)₂. In embodiments, R² is independently —OC(CH₃)₃. In embodiments, R² is independently —SCH₃. In embodiments, R² is independently —SCH₂CH₃. In embodiments, R² is independently —SCH₂CH₂CH₃. In embodiments, R² is independently —SCH(CH₃)₂. In embodiments, R² is independently —SC(CH₃)₃. In embodiments, R² is independently —CH₃. In embodiments, R² is independently —CH₂CH₃. In embodiments, R² is independently —CHCH₂. In embodiments, R² is independently —CH₂CH₂CH₃. In embodiments, R² is independently —CH(CH₃)₂. In embodiments, R² is independently —C(CH₃)₃.

In embodiments, R² is independently —CX²₃. In embodiments, R² is independently —CHX²₂. In embodiments, R² is independently —CH₂X². In embodiments, R² is independently —C(O)R^{2A}. In embodiments, R² is independently —C(O)OR^{2A}. In embodiments, R² is independently —C(O)NR^{2A}R^{2B}. In embodiments, R² is independently —COOH. In embodiments, R² is independently —CONH₂. In embodiments, R² is independently —CF₃. In embodiments, R² is independently —CHF₂. In embodiments, R² is independently —CH₂F. In embodiments, R² is independently —CH₃. In embodiments, R² is independently —CH₂CH₃. In embodiments, R² is independently —CH₂CH₂CH₃. In embodiments, R² is independently —CH(CH₃)₂. In embodiments, R² is independently —C(CH₃)₃. In embodiments, R² is independently —C(O)CF₃. In embodiments, R² is independently —C(O)CHF₂. In embodiments, R² is independently —C(O)CH₂F. In embodiments, R² is independently —C(O)CH₃. In embodiments, R² is independently —C(O)CH₂CH₃. In embodiments, R² is independently —C(O)CH₂CH₂CH₃. In embodiments, R² is independently —C(O)CH(CH₃)₂. In embodiments, R² is independently —C(O)C(CH₃)₃.

In embodiments, R² is independently halogen, —CX²₃, —CHX²₂, —CH₂X², —OCX²₃, —OCH₂X², —OCHX²₂, —OH, substituted or unsubstituted C₁-C₄ alkyl, or substituted or unsubstituted 2 to 4 membered heteroalkyl. In embodiments, R² is independently halogen, —OCH₃, —OH, or unsubstituted C₁-C₄ alkyl.

In embodiments, R² is independently oxo, halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OR³, —SR³, —NHR³, R³-substituted or unsubstituted alkyl (e.g., C₁-C₈, C₁-C₆, C₁-C₄, or C₁-C₂), R³-substituted or unsubstituted heteroalkyl (e.g., 2 to 8 membered, 2 to 6 membered, 4 to 6 membered, 2 to 3 membered, or 4 to 5 membered), R³-substituted or unsubstituted cycloalkyl (e.g., C₃-C₈, C₃-C₆, C₄-C₆, or C₅-C₆), R³-substituted or unsubstituted heterocycloalkyl (e.g., 3 to 8 membered, 3 to 6 membered, 4 to 6 membered, 4 to 5 membered, or 5 to 6 membered), R³-substituted or unsubstituted aryl (e.g., C₆-C₁₂, C₆-C₁₀, or phenyl), or R³-substituted or unsubstituted heteroaryl (e.g., 5 to 12 membered, 5 to 10 membered, 5 to 9 membered, or 5 to 6 membered). In embodiments, R² is independently oxo, halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R³-substituted or unsubstituted alkyl (e.g., C₁-C₈, C₁-C₆, C₁-C₄, or C₁-C₂), R³-substituted or unsubstituted heteroalkyl (e.g., 2 to 8 membered, 2 to 6 membered, 4 to 6 membered, 2 to 3 membered, or 4 to 5 membered), R³-substituted or unsubstituted cycloalkyl (e.g., C₃-C₈, C₃-C₆, C₄-C₆, or C₅-C₆), R³-substituted or unsubstituted heterocycloalkyl (e.g., 3 to 8 membered, 3 to 6 membered, 4 to 6 membered, 4 to 5 membered, or 5 to 6 membered), R³-substituted or unsubstituted aryl (e.g., C₆-C₁₂, C₆-C₁₀, Or phenyl), or R³-substituted or unsubstituted heteroaryl (e.g., 5 to 12 membered, 5 to 10 membered, 5 to 9 membered, or 5 to 6 membered). In embodiments, R² is independently oxo, halogen, —CX²₃, —CHX²₂, —CH₂X², —OCX²₃, —OCH₂X², —OCHX²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X² is independently —F, —Cl, —Br, or —I. In embodiments, R² is independently unsubstituted methyl. In embodiments, R² is independently unsubstituted ethyl.

In embodiments, two R² substituents (e.g., directly connected to adjacent atoms of R¹) may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, two R² substituents (e.g., directly connected to adjacent atoms of R¹) may optionally be joined to form an R³-substituted or unsubstituted cycloalkyl (e.g., C₃-C₈, C₃-C₆, C₄-C₆, or C₅-C₆), R³-substituted or unsubstituted heterocycloalkyl (e.g., 3 to 8 membered, 3 to 6 membered, 4 to 6 membered, 4 to 5 membered, or 5 to 6 membered), R³-substituted or unsubstituted aryl (e.g., C₆-C₁₂, C₆-C₁₀, or phenyl), or R³-substituted or unsubstituted heteroaryl (e.g., 5 to 12 membered, 5 to 10 membered, 5 to 9 membered, or 5 to 6 membered). In embodiments, two R² substituents (e.g., directly connected to adjacent atoms of R¹) may optionally be joined to form an unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered)

R³ is independently oxo,

halogen, —CX³³, —CHX³², —CH₂X³, —OCX³³, —OCH₂X³, —OCHX³², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R⁴-substituted or unsubstituted alkyl (e.g., C₁-C₈, C₁-C₆, C₁-C₄, or C₁-C₂), R⁴-substituted or unsubstituted heteroalkyl (e.g., 2 to 8 membered, 2 to 6 membered, 4 to 6 membered, 2 to 3 membered, or 4 to 5 membered), R⁴-substituted or unsubstituted cycloalkyl (e.g., C₃-C₈, C₃-C₆, C₄-C₆, or C₅-C₆), R⁴-substituted or unsubstituted heterocycloalkyl (e.g., 3 to 8 membered, 3 to 6 membered, 4 to 6 membered, 4 to 5 membered, or 5 to 6 membered), R⁴-substituted or unsubstituted aryl (e.g., C₆-C₁₂, C₆-C₁₀, or phenyl), or R⁴-substituted or unsubstituted heteroaryl (e.g., 5 to 12 membered, 5 to 10 membered, 5 to 9 membered, or 5 to 6 membered). In embodiments, R³ is independently oxo,
halogen, —CX³³, —CHX³², —CH₂X³, —OCX³³, —OCH₂X³, —OCHX³², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R⁴-substituted or unsubstituted alkyl (e.g., C₁-C₈, C₁-C₆, C₁-C₄, or C₁-C₂), R⁴-substituted or unsubstituted heteroalkyl (e.g., 2 to 8 membered, 2 to 6 membered, 4 to 6 membered, 2 to 3 membered, or 4 to 5 membered), R⁴-substituted or unsubstituted cycloalkyl (e.g., C₃-C₈, C₃-C₆, C₄-C₆, or C₅-C₆), R⁴-substituted or unsubstituted heterocycloalkyl (e.g., 3 to 8 membered, 3 to 6 membered, 4 to 6 membered, 4 to 5 membered, or 5 to 6 membered), R⁴-substituted or unsubstituted aryl (e.g., C₆-C₁₂, C₆-C₁₀, or phenyl), or R⁴-substituted or unsubstituted heteroaryl (e.g., 5 to 12 membered, 5 to 10 membered, 5 to 9 membered, or 5 to 6 membered). In embodiments, R³ is independently oxo,
halogen, —CX³³, —CHX³², —CH₂X³, —OCX³³, —OCH₂X³, —OCHX³², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X³ is independently —F, —Cl, —Br, or —I. In embodiments, R³ is independently unsubstituted methyl. In embodiments, R³ is independently unsubstituted ethyl.

R⁴ is independently oxo,

halogen, —CX⁴³, —CHX⁴², —CH₂X⁴, —OCX⁴³, —OCH₂X⁴, —OCHX⁴², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R⁵-substituted or unsubstituted alkyl (e.g., C₁-C₈, C₁-C₆, C₁-C₄, or C₁-C₂), R⁵-substituted or unsubstituted heteroalkyl (e.g., 2 to 8 membered, 2 to 6 membered, 4 to 6 membered, 2 to 3 membered, or 4 to 5 membered), R⁵-substituted or unsubstituted cycloalkyl (e.g., C₃-C₈, C₃-C₆, C₄-C₆, or C₅-C₆), R⁵-substituted or unsubstituted heterocycloalkyl (e.g., 3 to 8 membered, 3 to 6 membered, 4 to 6 membered, 4 to 5 membered, or 5 to 6 membered), R⁵-substituted or unsubstituted aryl (e.g., C₆-C₁₂, C₆-C₁₀, or phenyl), or R⁵-substituted or unsubstituted heteroaryl (e.g., 5 to 12 membered, 5 to 10 membered, 5 to 9 membered, or 5 to 6 membered). In embodiments, R⁴ is independently oxo,
halogen, —CX⁴³, —CHX⁴², —CH₂X⁴, —OCX⁴³, —OCH₂X⁴, —OCHX⁴², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R⁵-substituted or unsubstituted alkyl (e.g., C₁-C₈, C₁-C₆, C₁-C₄, or C₁-C₂), R⁵-substituted or unsubstituted heteroalkyl (e.g., 2 to 8 membered, 2 to 6 membered, 4 to 6 membered, 2 to 3 membered, or 4 to 5 membered), R⁵-substituted or unsubstituted cycloalkyl (e.g., C₃-C₈, C₃-C₆, C₄-C₆, or C₅-C₆), R⁵-substituted or unsubstituted heterocycloalkyl (e.g., 3 to 8 membered, 3 to 6 membered, 4 to 6 membered, 4 to 5 membered, or 5 to 6 membered), R⁵-substituted or unsubstituted aryl (e.g., C₆-C₁₂, C₆-C₁₀, or phenyl), or R⁵-substituted or unsubstituted heteroaryl (e.g., 5 to 12 membered, 5 to 10 membered, 5 to 9 membered, or 5 to 6 membered). In embodiments, R⁴ is independently oxo,
halogen, —CX⁴³, —CHX⁴², —CH₂X⁴, —OCX⁴³, —OCH₂X⁴, —OCHX⁴², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X⁴ is independently —F, —Cl, —Br, or —I. In embodiments, R⁴ is independently unsubstituted methyl. In embodiments, R⁴ is independently unsubstituted ethyl.

R⁵ is independently oxo,

halogen, —CX⁵³, —CHX⁵², —CH₂X⁵, —OCX⁵³, —OCH₂X⁵, —OCHX⁵², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R⁵ is independently oxo,
halogen, —CX⁵₃, —CHX⁵₂, —CH₂X⁵, —OCX⁵₃, —OCH₂X⁵, —OCHX⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X⁵ is independently —F, —Cl, —Br, or —I. In embodiments, R⁵ is independently unsubstituted methyl. In embodiments, R⁵ is independently unsubstituted ethyl.

In embodiments, R^{2A} is independently hydrogen. In embodiments, R^{2A} is independently —CX^{2A}₃. In embodiments, R^{2A} is independently —CHX^{2A}₂. In embodiments, R^{2A} is independently —CH₂X^{2A}. In embodiments, R^{2A} is independently —CN. In embodiments, R^{2A} is independently —COOH. In embodiments, R^{2A} is independently —CONH₂. In embodiments, X^{2A} is independently —F, —Cl, —Br, or —I.

In embodiments, R^{2A} is independently substituted or unsubstituted alkyl (e.g., C₁-C₈, C₁-C₆, C₁-C₄, or C₁-C₂). In embodiments, R^{2A} is independently substituted alkyl (e.g., C₁-C₈, C₁-C₆, C₁-C₄, or C₁-C₂). In embodiments, R^{2A} is independently unsubstituted alkyl (e.g., C₁-C₈, C₁-C₆, C₁-C₄, or C₁-C₂). In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted isopropyl. In embodiments, R^{2A} is independently unsubstituted tert-butyl. In embodiments, R^{2A} is independently substituted or unsubstituted heteroalkyl (e.g., 2 to 8 membered, 2 to 6 membered, 4 to 6 membered, 2 to 3 membered, or 4 to 5 membered). In embodiments, R^{2A} is independently substituted heteroalkyl (e.g., 2 to 8 membered, 2 to 6 membered, 4 to 6 membered, 2 to 3 membered, or 4 to 5 membered). In embodiments, R^{2A} is independently unsubstituted heteroalkyl (e.g., 2 to 8 membered, 2 to 6 membered, 4 to 6 membered, 2 to 3 membered, or 4 to 5 membered). In embodiments, R^{2A} is independently substituted or unsubstituted cycloalkyl (e.g., C₃-C₈, C₃-C₆, C₄-C₆, or C₅-C₆). In embodiments, R^{2A} is independently substituted cycloalkyl (e.g., C₃-C₈, C₃-C₆, C₄-C₆, or C₅-C₆). In embodiments, R^{2A} is independently unsubstituted cycloalkyl (e.g., C₃-C₈, C₃-C₆, C₄-C₆, or C₅-C₆). In embodiments, R^{2A} is independently substituted or unsubstituted heterocycloalkyl (e.g., 3 to 8 membered, 3 to 6 membered, 4 to 6 membered, 4 to 5 membered, or 5 to 6 membered). In embodiments, R^{2A} is independently substituted heterocycloalkyl (e.g., 3 to 8 membered, 3 to 6 membered, 4 to 6 membered, 4 to 5 membered, or 5 to 6 membered). In embodiments, R^{2A} is independently unsubstituted heterocycloalkyl (e.g., 3 to 8 membered, 3 to 6 membered, 4 to 6 membered, 4 to 5 membered, or 5 to 6 membered). In embodiments, R^{2A} is independently substituted or unsubstituted aryl (e.g., C₆-C₁₂, C₆-C₁₀, or phenyl). In embodiments, R^{2A} is independently substituted aryl (e.g., C₆-C₁₂, C₆-C₁₀, or phenyl). In embodiments, R^{2A} is independently unsubstituted aryl (e.g., C₆-C₁₂, C₆-C₁₀, or phenyl). In embodiments, R^{2A} is independently substituted or unsubstituted heteroaryl (e.g., 5 to 12 membered, 5 to 10 membered, 5 to 9 membered, or 5 to 6 membered). In embodiments, R^{2A} is independently substituted heteroaryl (e.g., 5 to 12 membered, 5 to 10 membered, 5 to 9 membered, or 5 to 6 membered). In embodiments, R^{2A} is independently unsubstituted heteroaryl (e.g., 5 to 12 membered, 5 to 10 membered, 5 to 9 membered, or 5 to 6 membered).

In embodiments, R^{2A} is independently

hydrogen, —CX^{2A}₃, —CHX^{2A}₂, —CH₂X^{2A}, —CN, —COOH, —CONH₂, R^{3A}-substituted or unsubstituted alkyl (e.g., C₁-C₈, C₁-C₆, C₁-C₄, or C₁-C₂), R^{3A}-substituted or unsubstituted heteroalkyl (e.g., 2 to 8 membered, 2 to 6 membered, 4 to 6 membered, 2 to 3 membered, or 4 to 5 membered), R^{3A}-substituted or unsubstituted cycloalkyl (e.g., C₃-C₈, C₃-C₆, C₄-C₆, or C₅-C₆), R^{3A}-substituted or unsubstituted heterocycloalkyl (e.g., 3 to 8 membered, 3 to 6 membered, 4 to 6 membered, 4 to 5 membered, or 5 to 6 membered), R^{3A}-substituted or unsubstituted aryl (e.g., C₆-C₁₂, C₆-C₁₀, or phenyl), or R^{3A}-substituted or unsubstituted heteroaryl (e.g., 5 to 12 membered, 5 to 10 membered, 5 to 9 membered, or 5 to 6 membered). In embodiments, R^{2A} is independently
hydrogen, —CX^{2A}₃, —CHX^{2A}₂, —CH₂X^{2A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2A} is independently —F, —Cl, —Br, or —I. In embodiments, R^{2A} is independently hydrogen. In embodiments, R^{2A} is independently unsubstituted methyl. In embodiments, R^{2A} is independently unsubstituted ethyl.

In embodiments, R^{2A} and R^{2B} substituents bonded to the same nitrogen atom may optionally be joined to form a R^{3A}-substituted or unsubstituted heterocycloalkyl (e.g., 3 to 8 membered, 3 to 6 membered, 4 to 6 membered, 4 to 5 membered, or 5 to 6 membered) or R^{3A}-substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form a R^{3A}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{3A} is independently oxo,

halogen, —CX^{3A}₃, —CHX^{3A}₂, —CH₂X^{3A}, —OCX^{3A}₃, —OCH₂X^{3A}, —OCHX^{3A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{4A}-substituted or unsubstituted alkyl (e.g., C₁-C₈, C₁-C₆, C₁-C₄, or C₁-C₂), R^{4A}-substituted or unsubstituted heteroalkyl (e.g., 2 to 8 membered, 2 to 6 membered, 4 to 6 membered, 2 to 3 membered, or 4 to 5 membered), R^{4A}-substituted or unsubstituted cycloalkyl (e.g., C₃-C₈, C₃-C₆, C₄-C₆, or C₅-C₆), R^{4A}-substituted or unsubstituted heterocycloalkyl (e.g., 3 to 8 membered, 3 to 6 membered, 4 to 6 membered, 4 to 5 membered, or 5 to 6 membered), R^{4A}-substituted or unsubstituted aryl (e.g., C₆-C₁₂, C₆-C₁₀, or phenyl), or R^{4A}-substituted or unsubstituted heteroaryl (e.g., 5 to 12 membered, 5 to 10 membered, 5 to 9 membered, or 5 to 6 membered). In embodiments, R^{3A} is independently oxo,
halogen, —CX^{3A}₃, —CHX^{3A}₂, —CH₂X^{3A}, —OCX^{3A}₃, —OCH₂X^{3A}, —OCHX^{3A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{4A}-substituted or unsubstituted alkyl (e.g., C₁-C₈, C₁-C₆, C₁-C₄, or C₁-C₂), R^{4A}-substituted or unsubstituted heteroalkyl (e.g., 2 to 8 membered, 2 to 6 membered, 4 to 6 membered, 2 to 3 membered, or 4 to 5 membered), R^{4A}-substituted or unsubstituted cycloalkyl (e.g., C₃-C₈, C₃-C₆, C₄-C₆, or C₅-C₆), R^{4A}-substituted or unsubstituted heterocycloalkyl (e.g., 3 to 8 membered, 3 to 6 membered, 4 to 6 membered, 4 to 5 membered, or 5 to 6 membered), R^{4A}-substituted or unsubstituted aryl (e.g., C₆-C₁₂, C₆-C₁₀, or phenyl), or R^{4A}-substituted or unsubstituted heteroaryl (e.g., 5 to 12 membered, 5 to 10 membered, 5 to 9 membered, or 5 to 6 membered). In embodiments, R^{3A} is independently oxo,
halogen, —CX^{3A}₃, —CHX^{3A}₂, —CH₂X^{3A}, —OCX^{3A}₃, —OCH₂X^{3A}, —OCHX^{3A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{3A} is independently —F, —Cl, —Br, or —I. In embodiments, R^{3A} is independently unsubstituted methyl. In embodiments, R^{3A} is independently unsubstituted ethyl.

R^{4A} is independently oxo,

halogen, —CX^{4A}₃, —CHX^{4A}₂, —CH₂X^{4A}, —OCX^{4A}₃, —OCH₂X^{4A}, —OCHX^{4A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{5A}-substituted or unsubstituted alkyl (e.g., C₁-C₈, C₁-C₆, C₁-C₄, or C₁-C₂), R^{5A}-substituted or unsubstituted heteroalkyl (e.g., 2 to 8 membered, 2 to 6 membered, 4 to 6 membered, 2 to 3 membered, or 4 to 5 membered), R^{5A}-substituted or unsubstituted cycloalkyl (e.g., C₃-C₈, C₃-C₆, C₄-C₆, or C₅-C₆), R^{5A}-substituted or unsubstituted heterocycloalkyl (e.g., 3 to 8 membered, 3 to 6 membered, 4 to 6 membered, 4 to 5 membered, or 5 to 6 membered), R^{5A}-substituted or unsubstituted aryl (e.g., C₆-C₁₂, C₆-C₁₀, or phenyl), or R^{5A}-substituted or unsubstituted heteroaryl (e.g., 5 to 12 membered, 5 to 10 membered, 5 to 9 membered, or 5 to 6 membered). In embodiments, R^{4A} is independently oxo,
halogen, —CX^{4A}₃, —CHX^{4A}₂, —CH₂X^{4A}, —OCX^{4A}₃, —OCH₂X^{4A}, —OCHX^{4A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{5A}-substituted or unsubstituted alkyl (e.g., C₁-C₈, C₁-C₆, C₁-C₄, or C₁-C₂), R^{5A}-substituted or unsubstituted heteroalkyl (e.g., 2 to 8 membered, 2 to 6 membered, 4 to 6 membered, 2 to 3 membered, or 4 to 5 membered), R^{5A}-substituted or unsubstituted cycloalkyl (e.g., C₃-C₈, C₃-C₆, C₄-C₆, or C₅-C₆), R^{5A}-substituted or unsubstituted heterocycloalkyl (e.g., 3 to 8 membered, 3 to 6 membered, 4 to 6 membered, 4 to 5 membered, or 5 to 6 membered), R^{5A}-substituted or unsubstituted aryl (e.g., C₆-C₁₂, C₆-C₁₀, or phenyl), or R^{5A}-substituted or unsubstituted heteroaryl (e.g., 5 to 12 membered, 5 to 10 membered, 5 to 9 membered, or 5 to 6 membered). In embodiments, R^{4A} is independently oxo,
halogen, —CX^{4A}₃, —CHX^{4A}₂, —CH₂X^{4A}, —OCX^{4A}₃, —OCH₂X^{4A}, —OCHX^{4A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{4A} is independently —F, —Cl, —Br, or —I. In embodiments, R^{4A} is independently unsubstituted methyl. In embodiments, R^{4A} is independently unsubstituted ethyl.

R^{5A} is independently oxo,

halogen, —CX^{5A}₃, —CHX^{5A}₂, —CH₂X^{5A}, —OCX^{5A}₃, —OCH₂X^{5A}, —OCHX^{5A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{5A} is independently oxo,
halogen, —CX^{5A}₃, —CHX^{5A}₂, —CH₂X^{5A}, —OCX^{5A}₃, —OCH₂X^{5A}, —OCHX^{5A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{5A} is independently —F, —Cl, —Br, or —I. In embodiments, R^{5A} is independently unsubstituted methyl. In embodiments, R^{5A} is independently unsubstituted ethyl.

In embodiments, R^{2B} is independently hydrogen. In embodiments, R^{2B} is independently —CX^{2B3}. In embodiments, R^{2B} is independently —CHX^{2B2}. In embodiments, R^{2B} is independently —CH₂X^{2B}. In embodiments, R^{2B} is independently —CN. In embodiments, R^{2B} is independently —COOH. In embodiments, R^{2B} is independently —CONH₂. In embodiments, X^{2B} is independently —F, —Cl, —Br, or —I.

In embodiments, R^{2B} is independently substituted or unsubstituted alkyl (e.g., C₁-C₈, C₁-C₆, C₁-C₄, or C₁-C₂). In embodiments, R^{2B} is independently substituted alkyl (e.g., C₁-C₈, C₁-C₆, C₁-C₄, or C₁-C₂). In embodiments, R^{2B} is independently unsubstituted alkyl (e.g., C₁-C₈, C₁-C₆, C₁-C₄, or C₁-C₂). In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted isopropyl. In embodiments, R^{2B} is independently unsubstituted tert-butyl. In embodiments, R^{2B} is independently substituted or unsubstituted heteroalkyl (e.g., 2 to 8 membered, 2 to 6 membered, 4 to 6 membered, 2 to 3 membered, or 4 to 5 membered). In embodiments, R^{2B} is independently substituted heteroalkyl (e.g., 2 to 8 membered, 2 to 6 membered, 4 to 6 membered, 2 to 3 membered, or 4 to 5 membered). In embodiments, R^{2B} is independently unsubstituted heteroalkyl (e.g., 2 to 8 membered, 2 to 6 membered, 4 to 6 membered, 2 to 3 membered, or 4 to 5 membered). In embodiments, R^{2B} is independently substituted or unsubstituted cycloalkyl (e.g., C₃-C₈, C₃-C₆, C₄-C₆, or C₅-C₆). In embodiments, R^{2B} is independently substituted cycloalkyl (e.g., C₃-C₈, C₃-C₆, C₄-C₆, or C₅-C₆). In embodiments, R^{2B} is independently unsubstituted cycloalkyl (e.g., C₃-C₈, C₃-C₆, C₄-C₆, or C₅-C₆). In embodiments, R^{2B} is independently substituted or unsubstituted heterocycloalkyl (e.g., 3 to 8 membered, 3 to 6 membered, 4 to 6 membered, 4 to 5 membered, or 5 to 6 membered). In embodiments, R^{2B} is independently substituted heterocycloalkyl (e.g., 3 to 8 membered, 3 to 6 membered, 4 to 6 membered, 4 to 5 membered, or 5 to 6 membered). In embodiments, R^{2B} is independently unsubstituted heterocycloalkyl (e.g., 3 to 8 membered, 3 to 6 membered, 4 to 6 membered, 4 to 5 membered, or 5 to 6 membered). In embodiments, R^{2B} is independently substituted or unsubstituted aryl (e.g., C₆-C₁₂, C₆-C₁₀, or phenyl). In embodiments, R^{2B} is independently substituted aryl (e.g., C₆-C₁₂, C₆-C₁₀, or phenyl). In embodiments, R^{2B} is independently unsubstituted aryl (e.g., C₆-C₁₂, C₆-C₁₀, or phenyl). In embodiments, R^{2B} is independently substituted or unsubstituted heteroaryl (e.g., 5 to 12 membered, 5 to 10 membered, 5 to 9 membered, or 5 to 6 membered). In embodiments, R^{2B} is independently substituted heteroaryl (e.g., 5 to 12 membered, 5 to 10 membered, 5 to 9 membered, or 5 to 6 membered). In embodiments, R^{2B} is independently unsubstituted heteroaryl (e.g., 5 to 12 membered, 5 to 10 membered, 5 to 9 membered, or 5 to 6 membered).

In embodiments, R^{2A} and R^{2B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{2A} and R^{2B} substituents bonded to the same nitrogen atom may be joined to form a substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may be joined to form a substituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may be joined to form an unsubstituted heteroaryl (e.g., 5 to 12 membered, 5 to 10 membered, 5 to 9 membered, or 5 to 6 membered).

In embodiments, R^{2B} is independently

hydrogen, —CX^{2B3}, —CHX^{2B2}, —CH₂X^{2B}, —CN, —COOH, —CONH₂, R^{3B}-substituted or unsubstituted alkyl (e.g., C₁-C₈, C₁-C₆, C₁-C₄, or C₁-C₂), R^{3B}-substituted or unsubstituted heteroalkyl (e.g., 2 to 8 membered, 2 to 6 membered, 4 to 6 membered, 2 to 3 membered, or 4 to 5 membered), R^{3B}-substituted or unsubstituted cycloalkyl (e.g., C₃-C₈, C₃-C₆, C₄-C₆, or C₅-C₆), R^{3B}-substituted or unsubstituted heterocycloalkyl (e.g., 3 to 8 membered, 3 to 6 membered, 4 to 6 membered, 4 to 5 membered, or 5 to 6 membered), R^{3B}-substituted or unsubstituted aryl (e.g., C₆-C₁₂, C₆-C₁₀, or phenyl), or R^{3B}-substituted or unsubstituted heteroaryl (e.g., 5 to 12 membered, 5 to 10 membered, 5 to 9 membered, or 5 to 6 membered). In embodiments, R^{2B} is independently
hydrogen, —CX^{2B3}, —CHX^{2B2}, —CH₂X^{2B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2B} is independently —F, —Cl, —Br, or —I. In embodiments, R^{2B} is independently hydrogen. In embodiments, R^{2B} is independently unsubstituted methyl. In embodiments, R^{2B} is independently unsubstituted ethyl.

In embodiments, R^{2A} and R^{2B} substituents bonded to the same nitrogen atom may optionally be joined to form a R^{3B}-substituted or unsubstituted heterocycloalkyl (e.g., 3 to 8 membered, 3 to 6 membered, 4 to 6 membered, 4 to 5 membered, or 5 to 6 membered) or R^{3B}-substituted or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{2A} and R^{2B} substituents bonded to the same nitrogen atom may optionally be joined to form a R^{3B}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{3B} is independently oxo,

halogen, —CX^{3B}₃, —CHX^{3B}₂, —CH₂X^{3B}, —OCX^{3B}₃, —OCH₂X^{3B}, —OCHX^{3B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{4B}-substituted or unsubstituted alkyl (e.g., C₁-C₈, C₁-C₆, C₁-C₄, or C₁-C₂), R^{4B}-substituted or unsubstituted heteroalkyl (e.g., 2 to 8 membered, 2 to 6 membered, 4 to 6 membered, 2 to 3 membered, or 4 to 5 membered), R^{4B}-substituted or unsubstituted cycloalkyl (e.g., C₃-C₈, C₃-C₆, C₄-C₆, or C₅-C₆), R^{4B}-substituted or unsubstituted heterocycloalkyl (e.g., 3 to 8 membered, 3 to 6 membered, 4 to 6 membered, 4 to 5 membered, or 5 to 6 membered), R^{4B}-substituted or unsubstituted aryl (e.g., C₆-C₁₂, C₆-C₁₀, or phenyl), or R^{4B}-substituted or unsubstituted heteroaryl (e.g., 5 to 12 membered, 5 to 10 membered, 5 to 9 membered, or 5 to 6 membered). In embodiments, R^{3B} is independently oxo,
halogen, —CX^{3B}₃, —CHX^{3B}₂, —CH₂X^{3B}, —OCX^{3B}₃, —OCH₂X^{3B}, —OCHX^{3B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{4B}-substituted or unsubstituted alkyl (e.g., C₁-C₈, C₁-C₆, C₁-C₄, or C₁-C₂), R^{4B}-substituted or unsubstituted heteroalkyl (e.g., 2 to 8 membered, 2 to 6 membered, 4 to 6 membered, 2 to 3 membered, or 4 to 5 membered), R^{4B}-substituted or unsubstituted cycloalkyl (e.g., C₃-C₈, C₃-C₆, C₄-C₆, or C₅-C₆), R^{4B}-substituted or unsubstituted heterocycloalkyl (e.g., 3 to 8 membered, 3 to 6 membered, 4 to 6 membered, 4 to 5 membered, or 5 to 6 membered), R^{4B}-substituted or unsubstituted aryl (e.g., C₆-C₁₂, C₆-C₁₀, Or phenyl), or R^{4B}-substituted or unsubstituted heteroaryl (e.g., 5 to 12 membered, 5 to 10 membered, 5 to 9 membered, or 5 to 6 membered). In embodiments, R^{3B} is independently oxo,
halogen, —CX^{3B}₃, —CHX^{3B}₂, —CH₂X^{3B}, —OCX^{3B}₃, —OCH₂X^{3B}, —OCHX^{3B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{3B} is independently —F, —Cl, —Br, or —I. In embodiments, R^{3B} is independently unsubstituted methyl. In embodiments, R^{3B} is independently unsubstituted ethyl.

R^{4B} is independently oxo,

halogen, —CX^{4B}₃, —CHX^{4B}₂, —CH₂X^{4B}, —OCX^{4B}₃, —OCH₂X^{4B}, —OCHX^{4B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{5B}-substituted or unsubstituted alkyl (e.g., C₁-C₈, C₁-C₆, C₁-C₄, or C₁-C₂), R^{5B}-substituted or unsubstituted heteroalkyl (e.g., 2 to 8 membered, 2 to 6 membered, 4 to 6 membered, 2 to 3 membered, or 4 to 5 membered), R^{5B}-substituted or unsubstituted cycloalkyl (e.g., C₃-C₈, C₃-C₆, C₄-C₆, or C₅-C₆), R^{5B}-substituted or unsubstituted heterocycloalkyl (e.g., 3 to 8 membered, 3 to 6 membered, 4 to 6 membered, 4 to 5 membered, or 5 to 6 membered), R^{5B}-substituted or unsubstituted aryl (e.g., C₆-C₁₂, C₆-C₁₀, or phenyl), or R^{5B}-substituted or unsubstituted heteroaryl (e.g., 5 to 12 membered, 5 to 10 membered, 5 to 9 membered, or 5 to 6 membered). In embodiments, R^{4B} is independently oxo,
halogen, —CX^{4B}₃, —CHX^{4B}₂, —CH₂X^{4B}, —OCX^{4B}₃, —OCH₂X^{4B}, —OCHX^{4B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{5B}-substituted or unsubstituted alkyl (e.g., C₁-C₈, C₁-C₆, C₁-C₄, or C₁-C₂), R^{5B}-substituted or unsubstituted heteroalkyl (e.g., 2 to 8 membered, 2 to 6 membered, 4 to 6 membered, 2 to 3 membered, or 4 to 5 membered), R^{5B}-substituted or unsubstituted cycloalkyl (e.g., C₃-C₈, C₃-C₆, C₄-C₆, or C₅-C₆), R^{5B}-substituted or unsubstituted heterocycloalkyl (e.g., 3 to 8 membered, 3 to 6 membered, 4 to 6 membered, 4 to 5 membered, or 5 to 6 membered), R^{SB}-substituted or unsubstituted aryl (e.g., C₆-C₁₂, C₆-C₁₀, Or phenyl), or R^{5B}-substituted or unsubstituted heteroaryl (e.g., 5 to 12 membered, 5 to 10 membered, 5 to 9 membered, or 5 to 6 membered). In embodiments, R^{4B} is independently oxo,
halogen, —CX^{4B}₃, —CHX^{4B}₂, —CH₂X^{4B}, —OCX^{4B}₃, —OCH₂X^{4B}, —OCHX^{4B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{4B} is independently —F, —Cl, —Br, or —I. In embodiments, R^{4B} is independently unsubstituted methyl. In embodiments, R^{4B} is independently unsubstituted ethyl.

R^{5B} is independently oxo,

halogen, —CX^{5B}₃, —CHX^{5B}₂, —CH₂X^{5B}, —OCX^{5B}₃, —OCH₂X^{5B}, —OCHX^{5B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{5B} is independently oxo,
halogen, —CX^{5B}₃, —CHX^{5B}₂, —CH₂X^{5B}, —OCX^{5B}₃, —OCH₂X^{5B}, —OCHX^{5B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{5B} is independently —F, —Cl, —Br, or —I. In embodiments, R^{5B} is independently unsubstituted methyl. In embodiments, R^{5B} is independently unsubstituted ethyl.

In embodiments, R^{2C} is independently hydrogen. In embodiments, R^{2C} is independently —CX^{2C}₃. In embodiments, R^{2C} is independently —CHX²C₂. In embodiments, R^{2C} is independently —CH₂X^{2C}. In embodiments, R^{2C} is independently —CN. In embodiments, R^{2C} is independently —COOH. In embodiments, R^{2C} is independently —CONH₂. In embodiments, X^{2C} is independently —F, —Cl, —Br, or —I.

In embodiments, R^{2C} is independently substituted or unsubstituted alkyl (e.g., C₁-C₈, C₁-C₆, C₁-C₄, or C₁-C₂). In embodiments, R^{2C} is independently substituted alkyl (e.g., C₁-C₈, C₁-C₆, C₁-C₄, or C₁-C₂). In embodiments, R^{2C} is independently unsubstituted alkyl (e.g., C₁-C₈, C₁-C₆, C₁-C₄, or C₁-C₂). In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted isopropyl. In embodiments, R^{2C} is independently unsubstituted tert-butyl. In embodiments, R^{2C} is independently substituted or unsubstituted heteroalkyl (e.g., 2 to 8 membered, 2 to 6 membered, 4 to 6 membered, 2 to 3 membered, or 4 to 5 membered). In embodiments, R^{2C} is independently substituted heteroalkyl (e.g., 2 to 8 membered, 2 to 6 membered, 4 to 6 membered, 2 to 3 membered, or 4 to 5 membered). In embodiments, R^{2C} is independently unsubstituted heteroalkyl (e.g., 2 to 8 membered, 2 to 6 membered, 4 to 6 membered, 2 to 3 membered, or 4 to 5 membered). In embodiments, R^{2C} is independently substituted or unsubstituted cycloalkyl (e.g., C₃-C₈, C₃-C₆, C₄-C₆, or C₅-C₆). In embodiments, R^{2C} is independently substituted cycloalkyl (e.g., C₃-C₈, C₃-C₆, C₄-C₆, or C₅-C₆). In embodiments, R^{2C} is independently unsubstituted cycloalkyl (e.g., C₃-C₈, C₃-C₆, C₄-C₆, or C₅-C₆). In embodiments, R^{2C} is independently substituted or unsubstituted heterocycloalkyl (e.g., 3 to 8 membered, 3 to 6 membered, 4 to 6 membered, 4 to 5 membered, or 5 to 6 membered). In embodiments, R^{2C} is independently substituted heterocycloalkyl (e.g., 3 to 8 membered, 3 to 6 membered, 4 to 6 membered, 4 to 5 membered, or 5 to 6 membered). In embodiments, R^{2C} is independently unsubstituted heterocycloalkyl (e.g., 3 to 8 membered, 3 to 6 membered, 4 to 6 membered, 4 to 5 membered, or 5 to 6 membered). In embodiments, R^{2C} is independently substituted or unsubstituted aryl (e.g., C₆-C₁₂, C₆-C₁₀, or phenyl). In embodiments, R^{2C} is independently substituted aryl (e.g., C₆-C₁₂, C₆-C₁₀, or phenyl). In embodiments, R^{2C} is independently unsubstituted aryl (e.g., C₆-C₁₂, C₆-C₁₀, or phenyl). In embodiments, R^{2C} is independently substituted or unsubstituted heteroaryl (e.g., 5 to 12 membered, 5 to 10 membered, 5 to 9 membered, or 5 to 6 membered). In embodiments, R^{2C} is independently substituted heteroaryl (e.g., 5 to 12 membered, 5 to 10 membered, 5 to 9 membered, or 5 to 6 membered). In embodiments, R^{2C} is independently unsubstituted heteroaryl (e.g., 5 to 12 membered, 5 to 10 membered, 5 to 9 membered, or 5 to 6 membered).

In embodiments, R^{2C} is independently

hydrogen, —CX^{2C}₃, —CHX^{2C2}, —CH₂X^{2C}, —CN, —COOH, —CONH₂, R^{3C}-substituted or unsubstituted alkyl (e.g., C₁-C₈, C₁-C₆, C₁-C₄, or C₁-C₂), R^{3C}-substituted or unsubstituted heteroalkyl (e.g., 2 to 8 membered, 2 to 6 membered, 4 to 6 membered, 2 to 3 membered, or 4 to 5 membered), R^{3C}-substituted or unsubstituted cycloalkyl (e.g., C₃-C₈, C₃-C₆, C₄-C₆, or C₅-C₆), R^{3C}-substituted or unsubstituted heterocycloalkyl (e.g., 3 to 8 membered, 3 to 6 membered, 4 to 6 membered, 4 to 5 membered, or 5 to 6 membered), R^{3C}-substituted or unsubstituted aryl (e.g., C₆-C₁₂, C₆-C₁₀, or phenyl), or R^{3C}-substituted or unsubstituted heteroaryl (e.g., 5 to 12 membered, 5 to 10 membered, 5 to 9 membered, or 5 to 6 membered). In embodiments, R^{2C} is independently
hydrogen, —CX^{2C}₃, —CHX^{2C2}, —CH₂X^{2C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{2C} is independently —F, —Cl, —Br, or —I. In embodiments, R^{2C} is independently hydrogen. In embodiments, R^{2C} is independently unsubstituted methyl. In embodiments, R^{2C} is independently unsubstituted ethyl.

R^{3C} is independently oxo,

halogen, —CX³C₃, —CHX³C₂, —CH₂X^{3C}, —OCX³C₃, —OCH₂X^{3C}, —OCHX³C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{4C}-substituted or unsubstituted alkyl (e.g., C₁-C₈, C₁-C₆, C₁-C₄, or C₁-C₂), R^{4C}-substituted or unsubstituted heteroalkyl (e.g., 2 to 8 membered, 2 to 6 membered, 4 to 6 membered, 2 to 3 membered, or 4 to 5 membered), R^{4C}-substituted or unsubstituted cycloalkyl (e.g., C₃-C₈, C₃-C₆, C₄-C₆, or C₅-C₆), R^{4C}-substituted or unsubstituted heterocycloalkyl (e.g., 3 to 8 membered, 3 to 6 membered, 4 to 6 membered, 4 to 5 membered, or 5 to 6 membered), R^{4C}-substituted or unsubstituted aryl (e.g., C₆-C₁₂, C₆-C₁₀, or phenyl), or R^{4C}-substituted or unsubstituted heteroaryl (e.g., 5 to 12 membered, 5 to 10 membered, 5 to 9 membered, or 5 to 6 membered). In embodiments, R^{3C} is independently oxo,
halogen, —CX³C₃, —CHX³C₂, —CH₂X^{3C}, —OCX³C₃, —OCH₂X^{3C}, —OCHX³C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{4C}-substituted or unsubstituted alkyl (e.g., C₁-C₈, C₁-C₆, C₁-C₄, or C₁-C₂), R^{4C}-substituted or unsubstituted heteroalkyl (e.g., 2 to 8 membered, 2 to 6 membered, 4 to 6 membered, 2 to 3 membered, or 4 to 5 membered), R^{4C}-substituted or unsubstituted cycloalkyl (e.g., C₃-C₈, C₃-C₆, C₄-C₆, or C₅-C₆), R^{4C}-substituted or unsubstituted heterocycloalkyl (e.g., 3 to 8 membered, 3 to 6 membered, 4 to 6 membered, 4 to 5 membered, or 5 to 6 membered), R^{4C}-substituted or unsubstituted aryl (e.g., C₆-C₁₂, C₆-C₁₀, or phenyl), or R^{4C}-substituted or unsubstituted heteroaryl (e.g., 5 to 12 membered, 5 to 10 membered, 5 to 9 membered, or 5 to 6 membered). In embodiments, R^{3C} is independently oxo,
halogen, —CX³C₃, —CHX³C₂, —CH₂X³C, —OCX³C₃, —OCH₂X³C, —OCHX³C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{3C} is independently —F, —Cl, —Br, or —I. In embodiments, R^{3C} is independently unsubstituted methyl. In embodiments, R^{3C} is independently unsubstituted ethyl.

R^{4C} is independently oxo,

halogen, —CX⁴C₃, —CHX⁴C₂, —CH₂X^{4C}, —OCX⁴C₃, —OCH₂X^{4C}, —OCHX⁴C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{5C}-substituted or unsubstituted alkyl (e.g., C₁-C₈, C₁-C₆, C₁-C₄, or C₁-C₂), R^{5C}-substituted or unsubstituted heteroalkyl (e.g., 2 to 8 membered, 2 to 6 membered, 4 to 6 membered, 2 to 3 membered, or 4 to 5 membered), R^{5C}-substituted or unsubstituted cycloalkyl (e.g., C₃-C₈, C₃-C₆, C₄-C₆, or C₅-C₆), R^{5C}-substituted or unsubstituted heterocycloalkyl (e.g., 3 to 8 membered, 3 to 6 membered, 4 to 6 membered, 4 to 5 membered, or 5 to 6 membered), R^{5C}-substituted or unsubstituted aryl (e.g., C₆-C₁₂, C₆-C₁₀, or phenyl), or R^{5C}-substituted or unsubstituted heteroaryl (e.g., 5 to 12 membered, 5 to 10 membered, 5 to 9 membered, or 5 to 6 membered). In embodiments, R^{4C} is independently oxo,
halogen, —CX⁴C₃, —CHX⁴C₂, —CH₂X^{4C}, —OCX⁴C₃, —OCH₂X^{4C}, —OCHX⁴C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{5C}-substituted or unsubstituted alkyl (e.g., C₁-C₈, C₁-C₆, C₁-C₄, or C₁-C₂), R^{5C}-substituted or unsubstituted heteroalkyl (e.g., 2 to 8 membered, 2 to 6 membered, 4 to 6 membered, 2 to 3 membered, or 4 to 5 membered), R^{5C}-substituted or unsubstituted cycloalkyl (e.g., C₃-C₈, C₃-C₆, C₄-C₆, or C₅-C₆), R^{5C}-substituted or unsubstituted heterocycloalkyl (e.g., 3 to 8 membered, 3 to 6 membered, 4 to 6 membered, 4 to 5 membered, or 5 to 6 membered), R^{5C}-substituted or unsubstituted aryl (e.g., C₆-C₁₂, C₆-C₁₀, or phenyl), or R^{5C}-substituted or unsubstituted heteroaryl (e.g., 5 to 12 membered, 5 to 10 membered, 5 to 9 membered, or 5 to 6 membered). In embodiments, R^{4C} is independently oxo,
halogen, —CX⁴C₃, —CHX⁴C₂, —CH₂X⁴C, —OCX⁴C₃, —OCH₂X⁴C, —OCHX⁴C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{4C} is independently —F, —Cl, —Br, or —I. In embodiments, R^{4C} is independently unsubstituted methyl. In embodiments, R^{4C} is independently unsubstituted ethyl.

R^{5C} is independently oxo,

halogen, —CX^{5C}₃, —CHX^{5C}₂, —CH₂X^{5C}, —OCX^{5C}₃, —OCH₂X^{5C}, —OCHX^{5C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{5C} is independently oxo,
halogen, —CX^{5C}₃, —CHX^{5C}₂, —CH₂X^{5C}, —OCX^{5C}₃, —OCH₂X^{5C}, —OCHX^{5C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{5C} is independently —F, —Cl, —Br, or —I. In embodiments, R^{5C} is independently

In embodiments, R⁶ is unsubstituted C₁-C₃ alkyl. In embodiments, R⁶ is unsubstituted C₁-C₂ alkyl. In embodiments, R⁶ is unsubstituted methyl.

In embodiments, R⁷ is independently hydrogen, oxo, halogen, —CX⁷₃, —CHX⁷₂, —CH₂X⁷, —OCX⁷₃, —OCH₂X⁷, —OCHX⁷₂, —CN, —SOₙ₇R^{7A}, —SOᵥ₇NR^{7A}R^{7B}, —NR^{7C}C(O)NR^{7A}R^{7B}, —N(O)ₘ₇, —NR^{7A}R^{7B}, —NR^{7C} NR^{7A}R^{7B}, —C(O)R^{7A}, —C(O)OR^{7A}, —C(O)NR^{7A}R^{7B}, —C(O)NR^{7C}NR^{7A}R^{7B}, —OR^{7A}, —NR^{7A}SO₂R^{7B}, —NR^{7A}C(O)R^{7B}, —NR^{7A}C(O)OR^{7B}, —NR^{7A}OR^{7B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is independently hydrogen, oxo, halogen, —CX⁷₃, —CHX⁷₂, —CH₂X⁷, —OCX⁷₃, —OCH₂X⁷, —OCHX⁷₂, —CN, —SOₙ₇R^{7A}, —SOᵥ₇NR^{7A}R^{7B}, —NR^{7C}C(O)NR^{7A}R^{7B}, —N(O)ₘ₇, —NR^{7A}R^{7B}, —NR^{7C} NR^{7A}R^{7B}, —C(O)R^{7A}, —C(O)OR^{7A}, —C(O)NR^{7A}R^{7B}, —C(O)NR^{7C}NR^{7A}R^{7B}, —OR^{7A}, —NR^{7A}SO₂R^{7B}, —NR^{7A}C(O)R^{7B}, —NR^{7A}C(O)OR^{7B}, —NR^{7A}OR^{7B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is hydrogen. In embodiments, R⁷ is, together with the oxygen to which it is attached, a prodrug moiety. In embodiments, R⁷ is independently hydrogen, oxo,

halogen, —CX⁷₃, —CHX⁷₂, —CH₂X⁷, —OCX⁷₃, —OCH₂X⁷, —OCHX⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R⁸-substituted or unsubstituted alkyl (e.g., C₁-C₈, C₁-C₆, C₁-C₄, or C₁-C₂), R⁸-substituted or unsubstituted heteroalkyl (e.g., 2 to 8 membered, 2 to 6 membered, 4 to 6 membered, 2 to 3 membered, or 4 to 5 membered), R⁸-substituted or unsubstituted cycloalkyl (e.g., C₃-C₈, C₃-C₆, C₄-C₆, or C₅-C₆), R⁸-substituted or unsubstituted heterocycloalkyl (e.g., 3 to 8 membered, 3 to 6 membered, 4 to 6 membered, 4 to 5 membered, or 5 to 6 membered), R⁸-substituted or unsubstituted aryl (e.g., C₆-C₁₂, C₆-C₁₀, or phenyl), or R⁸-substituted or unsubstituted heteroaryl (e.g., 5 to 12 membered, 5 to 10 membered, 5 to 9 membered, or 5 to 6 membered).

In embodiments, R⁷ is hydrogen or, together with the oxygen to which it is attached, forms a prodrug moiety.

In embodiments, R⁷ is independently hydrogen, halogen, —CX⁷₃, —CHX⁷₂, —CH₂X⁷, —CN, —SOₙ₇R^{7A}, —SOᵥ₇NR^{7A}R^{7B}, —NR^{7C}C(O)NR^{7A}R^{7B}, —NR^{7A}R^{7B}, —NR^{7C}NR^{7A}R^{7B}, —C(O)R^{7A}, —C(O)OR^{7A}, —C(O)NR^{7A}R^{7B}, —C(O)NR^{7C}NR^{7A}R^{7B}, —NR^{7A}SO₂R^{7B}, —NR^{7A}C(O)R^{7B}, —NR^{7A}C(O)OR^{7B}, —NR^{7A}OR^{7B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is hydrogen. In embodiments, R⁷ is, together with the oxygen to which it is attached, a prodrug moiety. In embodiments, R⁷ is an unsubstituted methyl.

R^{7A}, R^{7B}, and R^{7C} are independently hydrogen, —CX₃, —CHX₂, —CH₂X, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl.

In embodiments, R⁷ is independently hydrogen, halogen, —CX⁷₃, —CHX⁷₂, —CH₂X⁷, —CN, —NH₂, —COOH, —CONH₂, —SH, —SO₃H, —SO₄H, —SO₂NH₂, —NHNH₂, —NHC═(O)NHNH₂, —NHC═(O)NH₂, —NHSO₂H, —NHC═(O)H, —NHC(O)—OH, —NHOH, R⁸-substituted or unsubstituted alkyl (e.g., C₁-C₈, C₁-C₆, C₁-C₄, or C₁-C₂), R⁸-substituted or unsubstituted heteroalkyl (e.g., 2 to 8 membered, 2 to 6 membered, 4 to 6 membered, 2 to 3 membered, or 4 to 5 membered), R⁸-substituted or unsubstituted cycloalkyl (e.g., C₃-C₈, C₃-C₆, C₄-C₆, or C₅-C₆), R⁸-substituted or unsubstituted heterocycloalkyl (e.g., 3 to 8 membered, 3 to 6 membered, 4 to 6 membered, 4 to 5 membered, or 5 to 6 membered), R⁸-substituted or unsubstituted aryl (e.g., C₆-C₁₂, C₆-C₁₀, or phenyl), or R⁸-substituted or unsubstituted heteroaryl (e.g., 5 to 12 membered, 5 to 10 membered, 5 to 9 membered, or 5 to 6 membered). In embodiments, R⁷ is independently hydrogen, halogen, —CX⁷₃, —CHX⁷₂, —CH₂X⁷, —CN, —NH₂, —COOH, —CONH₂, —SH, —SO₃H, —SO₄H, —SO₂NH₂, —NHNH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X⁷ is independently —F, —Cl, —Br, or —I. In embodiments, R⁷ is independently hydrogen.

R⁸ is independently oxo,

halogen, —CX⁸³, —CHX⁸², —CH₂X⁸, —OCX⁸³, —OCH₂X⁸, —OCHX⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R⁹-substituted or unsubstituted alkyl (e.g., C₁-C₈, C₁-C₆, C₁-C₄, or C₁-C₂), R⁹-substituted or unsubstituted heteroalkyl (e.g., 2 to 8 membered, 2 to 6 membered, 4 to 6 membered, 2 to 3 membered, or 4 to 5 membered), R⁹-substituted or unsubstituted cycloalkyl (e.g., C₃-C₈, C₃-C₆, C₄-C₆, or C₅-C₆), R⁹-substituted or unsubstituted heterocycloalkyl (e.g., 3 to 8 membered, 3 to 6 membered, 4 to 6 membered, 4 to 5 membered, or 5 to 6 membered), R⁹-substituted or unsubstituted aryl (e.g., C₆-C₁₂, C₆-C₁₀, or phenyl), or R⁹-substituted or unsubstituted heteroaryl (e.g., 5 to 12 membered, 5 to 10 membered, 5 to 9 membered, or 5 to 6 membered). In embodiments, R⁸ is independently oxo,
halogen, —CX⁸³, —CHX⁸², —CH₂X⁸, —OCX⁸³, —OCH₂X⁸, —OCHX⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R⁹-substituted or unsubstituted alkyl (e.g., C₁-C₈, C₁-C₆, C₁-C₄, or C₁-C₂), R⁹-substituted or unsubstituted heteroalkyl (e.g., 2 to 8 membered, 2 to 6 membered, 4 to 6 membered, 2 to 3 membered, or 4 to 5 membered), R⁹-substituted or unsubstituted cycloalkyl (e.g., C₃-C₈, C₃-C₆, C₄-C₆, or C₅-C₆), R⁹-substituted or unsubstituted heterocycloalkyl (e.g., 3 to 8 membered, 3 to 6 membered, 4 to 6 membered, 4 to 5 membered, or 5 to 6 membered), R⁹-substituted or unsubstituted aryl (e.g., C₆-C₁₂, C₆-C₁₀, or phenyl), or R⁹-substituted or unsubstituted heteroaryl (e.g., 5 to 12 membered, 5 to 10 membered, 5 to 9 membered, or 5 to 6 membered). In embodiments, R⁸ is independently oxo,
halogen, —CX⁸³, —CHX⁸², —CH₂X⁸, —OCX⁸³, —OCH₂X⁸, —OCHX⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X⁸ is independently —F, —Cl, —Br, or —I. In embodiments, R⁸ is independently unsubstituted methyl. In embodiments, R⁸ is independently unsubstituted ethyl.

R⁹ is independently oxo,

halogen, —CX⁹³, —CHX⁹², —CH₂X⁹, —OCX⁹³, —OCH₂X⁹, —OCHX⁹², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R¹⁰-substituted or unsubstituted alkyl (e.g., C₁-C₈, C₁-C₆, C₁-C₄, or C₁-C₂), R¹⁰-substituted or unsubstituted heteroalkyl (e.g., 2 to 8 membered, 2 to 6 membered, 4 to 6 membered, 2 to 3 membered, or 4 to 5 membered), R¹⁰-substituted or unsubstituted cycloalkyl (e.g., C₃-C₈, C₃-C₆, C₄-C₆, or C₅-C₆), R¹⁰-substituted or unsubstituted heterocycloalkyl (e.g., 3 to 8 membered, 3 to 6 membered, 4 to 6 membered, 4 to 5 membered, or 5 to 6 membered), R¹⁰-substituted or unsubstituted aryl (e.g., C₆-C₁₂, C₆-C₁₀, or phenyl), or R¹⁰-substituted or unsubstituted heteroaryl (e.g., 5 to 12 membered, 5 to 10 membered, 5 to 9 membered, or 5 to 6 membered). In embodiments, R⁹ is independently oxo,
halogen, —CX⁹³, —CHX⁹², —CH₂X⁹, —OCX⁹³, —OCH₂X⁹, —OCHX⁹², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R¹⁰-substituted or unsubstituted alkyl (e.g., C₁-C₈, C₁-C₆, C₁-C₄, or C₁-C₂), R¹⁰-substituted or unsubstituted heteroalkyl (e.g., 2 to 8 membered, 2 to 6 membered, 4 to 6 membered, 2 to 3 membered, or 4 to 5 membered), R¹⁰-substituted or unsubstituted cycloalkyl (e.g., C₃-C₈, C₃-C₆, C₄-C₆, or C₅-C₆), R¹⁰-substituted or unsubstituted heterocycloalkyl (e.g., 3 to 8 membered, 3 to 6 membered, 4 to 6 membered, 4 to 5 membered, or 5 to 6 membered), R¹⁰-substituted or unsubstituted aryl (e.g., C₆-C₁₂, C₆-C₁₀, or phenyl), or R¹⁰-substituted or unsubstituted heteroaryl (e.g., 5 to 12 membered, 5 to 10 membered, 5 to 9 membered, or 5 to 6 membered). In embodiments, R⁹ is independently oxo,
halogen, —CX⁹³, —CHX⁹², —CH₂X⁹, —OCX⁹³, —OCH₂X⁹, —OCHX⁹², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X⁹ is independently —F, —Cl, —Br, or —I. In embodiments, R⁹ is independently unsubstituted methyl. In embodiments, R⁹ is independently unsubstituted ethyl.

R¹⁰ is independently oxo,

halogen, —CX¹⁰₃, —CHX¹⁰₂, —CH₂X¹⁰, —OCX¹⁰³, —OCH₂X¹⁰, —OCHX¹⁰₂, —CN, —OH, —NH₂, —COOH, —C ONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R¹⁰ is independently oxo,
halogen, —CX¹⁰₃, —CHX¹⁰², —CH₂X¹⁰, —OCX¹⁰³, —OCH₂X¹⁰, —OCHX¹⁰², —CN, —OH, —NH₂, —COOH, —C ONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X¹⁰ is independently —F, —Cl, —Br, or —I. In embodiments, R¹⁰ is independently unsubstituted methyl. In embodiments, R¹⁰ is independently unsubstituted ethyl.

In embodiments, R^{7A} is independently hydrogen. In embodiments, R^{7A} is independently —CX^{7A3}. In embodiments, R^{7A} is independently —CHX^{7A2}. In embodiments, R^{7A} is independently —CH₂X^{7A}. In embodiments, R^{7A} is independently —CN. In embodiments, R^{7A} is independently —COOH. In embodiments, R^{7A} is independently —CONH₂. In embodiments, X^{7A} is independently —F, —Cl, —Br, or —I.

In embodiments, R^{7A} is independently substituted or unsubstituted alkyl (e.g., C₁-C₈, C₁-C₆, C₁-C₄, or C₁-C₂). In embodiments, R^{7A} is independently substituted alkyl (e.g., C₁-C₈, C₁-C₆, C₁-C₄, or C₁-C₂). In embodiments, R^{7A} is independently unsubstituted alkyl (e.g., C₁-C₈, C₁-C₆, C₁-C₄, or C₁-C₂). In embodiments, R^{7A} is independently unsubstituted methyl. In embodiments, R^{7A} is independently unsubstituted ethyl. In embodiments, R^{7A} is independently unsubstituted propyl. In embodiments, R^{7A} is independently unsubstituted isopropyl. In embodiments, R^{7A} is independently unsubstituted tert-butyl. In embodiments, R^{7A} is independently substituted or unsubstituted heteroalkyl (e.g., 2 to 8 membered, 2 to 6 membered, 4 to 6 membered, 2 to 3 membered, or 4 to 5 membered). In embodiments, R^{7A} is independently substituted heteroalkyl (e.g., 2 to 8 membered, 2 to 6 membered, 4 to 6 membered, 2 to 3 membered, or 4 to 5 membered). In embodiments, R^{7A} is independently unsubstituted heteroalkyl (e.g., 2 to 8 membered, 2 to 6 membered, 4 to 6 membered, 2 to 3 membered, or 4 to 5 membered). In embodiments, R^{7A} is independently substituted or unsubstituted cycloalkyl (e.g., C₃-C₈, C₃-C₆, C₄-C₆, or C₅-C₆). In embodiments, R^{7A} is independently substituted cycloalkyl (e.g., C₃-C₈, C₃-C₆, C₄-C₆, or C₅-C₆). In embodiments, R^{7A} is independently unsubstituted cycloalkyl (e.g., C₃-C₈, C₃-C₆, C₄-C₆, or C₅-C₆). In embodiments, R^{7A} is independently substituted or unsubstituted heterocycloalkyl (e.g., 3 to 8 membered, 3 to 6 membered, 4 to 6 membered, 4 to 5 membered, or 5 to 6 membered). In embodiments, R^{7A} is independently substituted heterocycloalkyl (e.g., 3 to 8 membered, 3 to 6 membered, 4 to 6 membered, 4 to 5 membered, or 5 to 6 membered). In embodiments, R^{7A} is independently unsubstituted heterocycloalkyl (e.g., 3 to 8 membered, 3 to 6 membered, 4 to 6 membered, 4 to 5 membered, or 5 to 6 membered). In embodiments, R^{7A} is independently substituted or unsubstituted aryl (e.g., C₆-C₁₂, C₆-C₁₀, or phenyl). In embodiments, R^{7A} is independently substituted aryl (e.g., C₆-C₁₂, C₆-C₁₀, or phenyl). In embodiments, R^{7A} is independently unsubstituted aryl (e.g., C₆-C₁₂, C₆-C₁₀, or phenyl). In embodiments, R^{7A} is independently substituted or unsubstituted heteroaryl (e.g., 5 to 12 membered, 5 to 10 membered, 5 to 9 membered, or 5 to 6 membered). In embodiments, R^{7A} is independently substituted heteroaryl (e.g., 5 to 12 membered, 5 to 10 membered, 5 to 9 membered, or 5 to 6 membered). In embodiments, R^{7A} is independently unsubstituted heteroaryl (e.g., 5 to 12 membered, 5 to 10 membered, 5 to 9 membered, or 5 to 6 membered). In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different.

In embodiments, R^{7B} is independently hydrogen. In embodiments, R^{7B} is independently —CX^{7B}₃. In embodiments, R^{7B} is independently —CHX^{7B}₂. In embodiments, R^{7B} is independently —CH₂X^{7B}. In embodiments, R^{7B} is independently —CN. In embodiments, R^{7B} is independently —COOH. In embodiments, R^{7B} is independently —CONH₂. In embodiments, X^{7B} is independently —F, —Cl, —Br, or —I.

In embodiments, R^{7B} is independently substituted or unsubstituted alkyl (e.g., C₁-C₈, C₁-C₆, C₁-C₄, or C₁-C₂). In embodiments, R^{7B} is independently substituted alkyl (e.g., C₁-C₈, C₁-C₆, C₁-C₄, or C₁-C₂). In embodiments, R^{7B} is independently unsubstituted alkyl (e.g., C₁-C₈, C₁-C₆, C₁-C₄, or C₁-C₂). In embodiments, R^{7B} is independently unsubstituted methyl. In embodiments, R^{7B} is independently unsubstituted ethyl. In embodiments, R^{7B} is independently unsubstituted propyl. In embodiments, R^{7B} is independently unsubstituted isopropyl. In embodiments, R^{7B} is independently unsubstituted tert-butyl. In embodiments, R^{7B} is independently substituted or unsubstituted heteroalkyl (e.g., 2 to 8 membered, 2 to 6 membered, 4 to 6 membered, 2 to 3 membered, or 4 to 5 membered). In embodiments, R^{7B} is independently substituted heteroalkyl (e.g., 2 to 8 membered, 2 to 6 membered, 4 to 6 membered, 2 to 3 membered, or 4 to 5 membered). In embodiments, R^{7B} is independently unsubstituted heteroalkyl (e.g., 2 to 8 membered, 2 to 6 membered, 4 to 6 membered, 2 to 3 membered, or 4 to 5 membered). In embodiments, R^{7B} is independently substituted or unsubstituted cycloalkyl (e.g., C₃-C₈, C₃-C₆, C₄-C₆, or C₅-C₆). In embodiments, R^{7B} is independently substituted cycloalkyl (e.g., C₃-C₈, C₃-C₆, C₄-C₆, or C₅-C₆). In embodiments, R^{7B} is independently unsubstituted cycloalkyl (e.g., C₃-C₈, C₃-C₆, C₄-C₆, or C₅-C₆). In embodiments, R^{7B} is independently substituted or unsubstituted heterocycloalkyl (e.g., 3 to 8 membered, 3 to 6 membered, 4 to 6 membered, 4 to 5 membered, or 5 to 6 membered). In embodiments, R^{7B} is independently substituted heterocycloalkyl (e.g., 3 to 8 membered, 3 to 6 membered, 4 to 6 membered, 4 to 5 membered, or 5 to 6 membered). In embodiments, R^{7B} is independently unsubstituted heterocycloalkyl (e.g., 3 to 8 membered, 3 to 6 membered, 4 to 6 membered, 4 to 5 membered, or 5 to 6 membered). In embodiments, R^{7B} is independently substituted or unsubstituted aryl (e.g., C₆-C₁₂, C₆-C₁₀, or phenyl). In embodiments, R^{7B} is independently substituted aryl (e.g., C₆-C₁₂, C₆-C₁₀, or phenyl). In embodiments, R^{7B} is independently unsubstituted aryl (e.g., C₆-C₁₂, C₆-C₁₀, or phenyl). In embodiments, R^{7B} is independently substituted or unsubstituted heteroaryl (e.g., 5 to 12 membered, 5 to 10 membered, 5 to 9 membered, or 5 to 6 membered). In embodiments, R^{7B} is independently substituted heteroaryl (e.g., 5 to 12 membered, 5 to 10 membered, 5 to 9 membered, or 5 to 6 membered). In embodiments, R^{7B} is independently unsubstituted heteroaryl (e.g., 5 to 12 membered, 5 to 10 membered, 5 to 9 membered, or 5 to 6 membered). In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different.

In embodiments, R^{7A} and R^{7B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{7A} and R^{7B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{7A} and R^{7B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{7A} and R^{7B} substituents bonded to the same nitrogen atom may be joined to form a substituted or unsubstituted heteroaryl (e.g., 5 to 12 membered, 5 to 10 membered, 5 to 9 membered, or 5 to 6 membered). In embodiments, R^{7A} and R^{7B} substituents bonded to the same nitrogen atom may be joined to form a substituted heteroaryl (e.g., 5 to 12 membered, 5 to 10 membered, 5 to 9 membered, or 5 to 6 membered). In embodiments, R^{7A} and R^{7B} substituents bonded to the same nitrogen atom may be joined to form an unsubstituted heteroaryl (e.g., 5 to 12 membered, 5 to 10 membered, 5 to 9 membered, or 5 to 6 membered).

In embodiments, a substituted joined R^{7A} and R^{7B} substituents (e.g., substituted heterocycloalkyl and/or substituted heteroaryl) is substituted with at least one substituent group, size-limited substituent group, or lower substituent group; wherein if the substituted joined R^{7A} and R^{7B} substituents is substituted with a plurality of groups selected from substituent groups, size-limited substituent groups, and lower substituent groups; each substituent group, size-limited substituent group, and/or lower substituent group may optionally be different.

In embodiments, R^{7C} is independently hydrogen. In embodiments, R^{7C} is independently —CX^{7C3}. In embodiments, R^{7C} is independently —CHX⁷C₂. In embodiments, R^{7C} is independently —CH₂X^{7C}. In embodiments, R^{7C} is independently —CN. In embodiments, R^{7C} is independently —COOH. In embodiments, R^{7C} is independently —CONH₂. In embodiments, X^{7C} is independently —F, —Cl, —Br, or —I.

In embodiments, R^{7C} is independently substituted or unsubstituted alkyl (e.g., C₁-C₈, C₁-C₆, C₁-C₄, or C₁-C₂). In embodiments, R^{7C} is independently substituted alkyl (e.g., C₁-C₈, C₁-C₆, C₁-C₄, or C₁-C₂). In embodiments, R^{7C} is independently unsubstituted alkyl (e.g., C₁-C₈, C₁-C₆, C₁-C₄, or C₁-C₂). In embodiments, R^{7C} is independently unsubstituted methyl. In embodiments, R^{7C} is independently unsubstituted ethyl. In embodiments, R^{7C} is independently unsubstituted propyl. In embodiments, R^{7C} is independently unsubstituted isopropyl. In embodiments, R^{7C} is independently unsubstituted tert-butyl. In embodiments, R^{7C} is independently substituted or unsubstituted heteroalkyl (e.g., 2 to 8 membered, 2 to 6 membered, 4 to 6 membered, 2 to 3 membered, or 4 to 5 membered). In embodiments, R^{7C} is independently substituted heteroalkyl (e.g., 2 to 8 membered, 2 to 6 membered, 4 to 6 membered, 2 to 3 membered, or 4 to 5 membered). In embodiments, R^{7C} is independently unsubstituted heteroalkyl (e.g., 2 to 8 membered, 2 to 6 membered, 4 to 6 membered, 2 to 3 membered, or 4 to 5 membered). In embodiments, R^{7C} is independently substituted or unsubstituted cycloalkyl (e.g., C₃-C₈, C₃-C₆, C₄-C₆, or C₅-C₆). In embodiments, R^{7C} is independently substituted cycloalkyl (e.g., C₃-C₈, C₃-C₆, C₄-C₆, or C₅-C₆). In embodiments, R^{7C} is independently unsubstituted cycloalkyl (e.g., C₃-C₈, C₃-C₆, C₄-C₆, or C₅-C₆). In embodiments, R^{7C} is independently substituted or unsubstituted heterocycloalkyl (e.g., 3 to 8 membered, 3 to 6 membered, 4 to 6 membered, 4 to 5 membered, or 5 to 6 membered). In embodiments, R^{7C} is independently substituted heterocycloalkyl (e.g., 3 to 8 membered, 3 to 6 membered, 4 to 6 membered, 4 to 5 membered, or 5 to 6 membered). In embodiments, R^{7C} is independently unsubstituted heterocycloalkyl (e.g., 3 to 8 membered, 3 to 6 membered, 4 to 6 membered, 4 to 5 membered, or 5 to 6 membered). In embodiments, R^{7C} is independently substituted or unsubstituted aryl (e.g., C₆-C₁₂, C₆-C₁₀, or phenyl). In embodiments, R^{7C} is independently substituted aryl (e.g., C₆-C₁₂, C₆-C₁₀, or phenyl). In embodiments, R^{7C} is independently unsubstituted aryl (e.g., C₆-C₁₂, C₆-C₁₀, or phenyl). In embodiments, R^{7C} is independently substituted or unsubstituted heteroaryl (e.g., 5 to 12 membered, 5 to 10 membered, 5 to 9 membered, or 5 to 6 membered). In embodiments, R^{7C} is independently substituted heteroaryl (e.g., 5 to 12 membered, 5 to 10 membered, 5 to 9 membered, or 5 to 6 membered). In embodiments, R^{7C} is independently unsubstituted heteroaryl (e.g., 5 to 12 membered, 5 to 10 membered, 5 to 9 membered, or 5 to 6 membered). In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different.

In embodiments, X is —F. In embodiments, X is —Cl. In embodiments, X is —Br. In embodiments, X is —I. In embodiments, X² is —F. In embodiments, X² is —Cl. In embodiments, X² is —Br. In embodiments, X² is —I. In embodiments, X⁷ is —F. In embodiments, X⁷ is —Cl. In embodiments, X⁷ is —Br. In embodiments, X⁷ is —I.

In embodiments, n2 is 0. In embodiments, n2 is 1. In embodiments, n2 is 2. In embodiments, n2 is 3. In embodiments, n2 is 4. In embodiments, n7 is 0. In embodiments, n7 is 1. In embodiments, n7 is 2. In embodiments, n7 is 3. In embodiments, n7 is 4.

In embodiments, m2 is 1. In embodiments, m2 is 2. In embodiments, m7 is 1. In embodiments, m7 is 2.

In embodiments, v2 is 1. In embodiments, v2 is 2. In embodiments, v7 is 1. In embodiments, v7 is 2.

In embodiments, the compound has the formula:

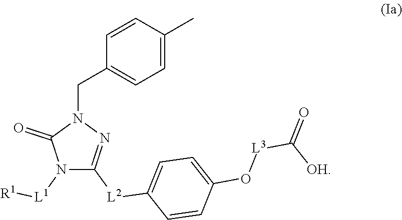

L¹, L², L³, and R¹ are as described herein.

In embodiments, the compound has the formula:

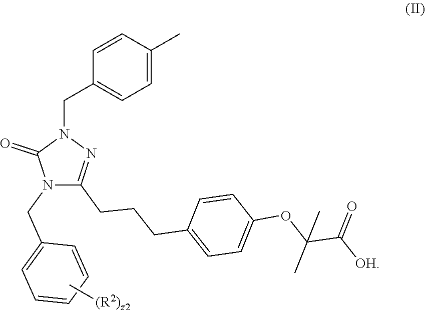

R² is as described herein.

z2 is an integer from 0 to 5.

In embodiments, z2 is 1. In embodiments, z2 is 2. In embodiments, z2 is 3. In embodiments, z2 is 4. In embodiments, z2 is 5.

In embodiments, the compound has the formula:

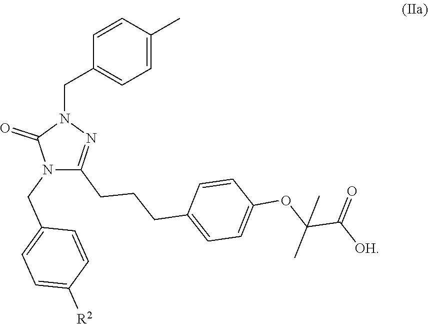

R² is as described herein.

In embodiments, the compound has the formula:

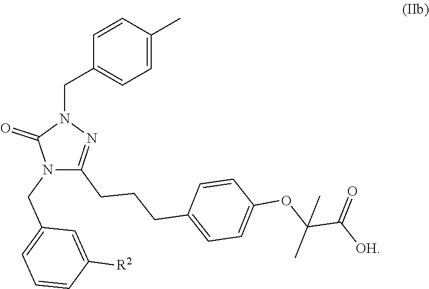

R² is as described herein.

In embodiments, the compound has the formula:

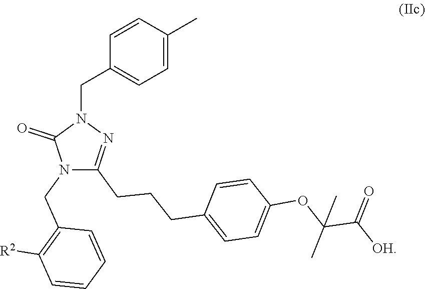

R² is as described herein.

In embodiments, the compound has the formula:

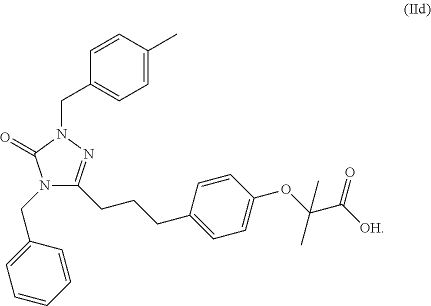 [SMILES: Cc1ccc(Cn2nc(CCCc3ccc(OC(C)(C)C(=O)O)cc3)n(Cc3ccccc3)c2=O)cc1]

In embodiments, the compound is selected from:

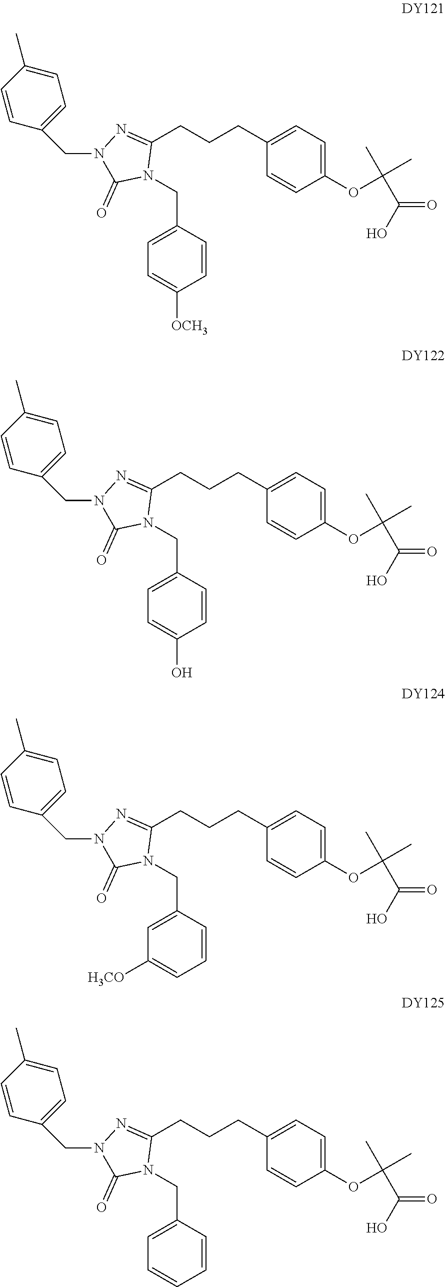 [SMILES: COc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(O)cc3)c2=O)cc1 ; COc1cccc(Cn2c(CCCc3ccc(OC(C)(C)C(=O)O)cc3)nn(Cc3ccc(C)cc3)c2=O)c1 ; Cc1ccc(Cn2nc(CCCc3ccc(OC(C)(C)C(=O)O)cc3)n(Cc3ccccc3)c2=O)cc1]

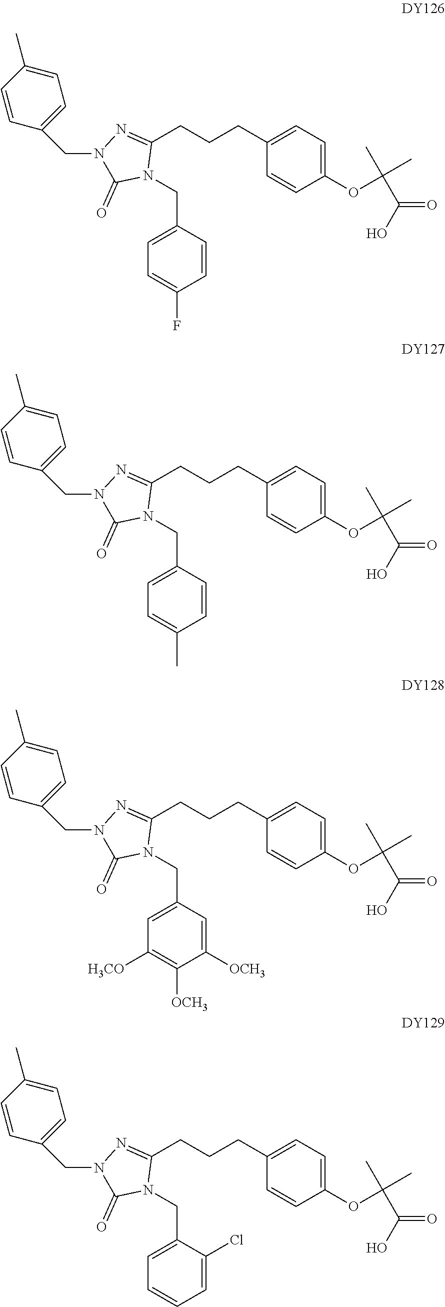 [SMILES: Cc1ccc(Cn2nc(CCCc3ccc(OC(C)(C)C(=O)O)cc3)n(Cc3ccc(F)cc3)c2=O)cc1 ; Cc1ccc(Cn2nc(CCCc3ccc(OC(C)(C)C(=O)O)cc3)n(Cc3ccc(C)cc3)c2=O)cc1 ; COc1cc(Cn2c(CCCc3ccc(OC(C)(C)C(=O)O)cc3)nn(Cc3ccc(C)cc3)c2=O)cc(OC)c1OC ; Cc1ccc(Cn2nc(CCCc3ccc(OC(C)(C)C(=O)O)cc3)n(Cc3ccccc3Cl)c2=O)cc1]

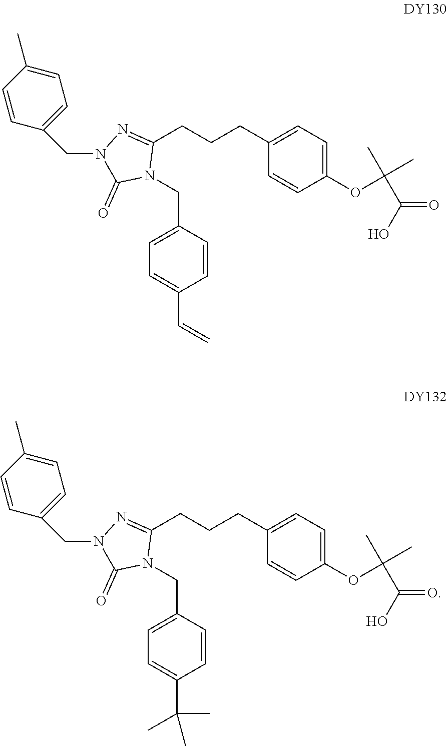 [SMILES: C=Cc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(C(C)(C)C)cc3)c2=O)cc1]

In embodiments, the compound has the formula:

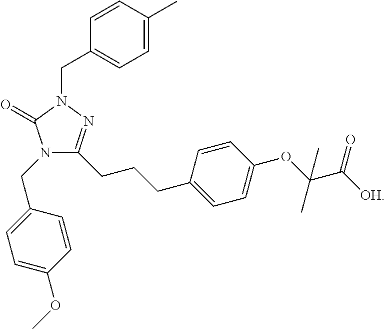 [SMILES: COc1ccc(Cn2c(CCCc3ccc(OC(C)(C)C(=O)O)cc3)nn(Cc3ccc(C)cc3)c2=O)cc1]

In some embodiments, a compound as described herein may include multiple instances of R² and/or other variables. In such embodiments, each variable may optional be different and be appropriately labeled to distinguish each group for greater clarity. For example, where each R² is different, they may be referred to, for example, as R^{2.1}, R^{2.2}, R^{2.3}, R^{2.4}, R^{2.5}, respectively, wherein the definition of R² is assumed by R^{2.1}, R^{2.2}, R^{2.3}, R^{2.4}, R^{2.5}. The variables used within a definition of R² and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

In embodiments, the compound has the formula:

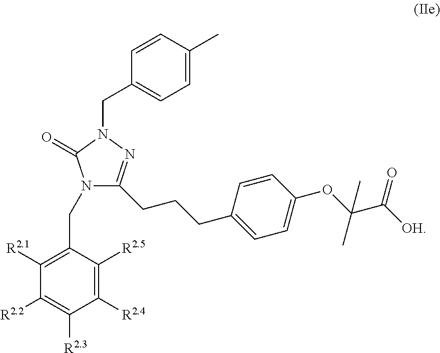

R^{2.1}, R^{2.2}, R^{2.3}, R^{2.4}, and R^{2.5} are independently hydrogen or any value of R².

In embodiments, R^{2.1} is independently hydrogen. In embodiments, R^{2.1} is independently oxo. In embodiments, R^{2.1} is independently halogen. In embodiments, R^{2.1} is independently —CX²₃. In embodiments, R^{2.1} is independently —CHX²₂. In embodiments, R^{2.1} is independently —CH₂X². In embodiments, R^{2.1} is independently —OCX²₃. In embodiments, R^{2.1} is independently —OCH₂X². In embodiments, R^{2.1} is independently —OCHX²₂. In embodiments, R^{2.1} is independently —CN. In embodiments, R^{2.1} is independently —SOₙ₂R^{2A}. In embodiments, R^{2.1} is independently —SOᵥ₂NR^{2A}R^{2B}. In embodiments, R^{2.1} is independently —NR^{2C}C(O)NR^{2A}R^{2B}. In embodiments, R^{2.1} is independently —N(O)ₘ₂. In embodiments, R^{2.1} is independently —NR^{2A}R^{2B}. In embodiments, R^{2.1} is independently —NR^{2C}NR^{2A}R^{2B}. In embodiments, R^{2.1} is independently —C(O)R^{2A}. In embodiments, R^{2.1} is independently —C(O)OR^{2A}. In embodiments, R^{2.1} is independently —C(O)NR^{2A}R^{2B}. In embodiments, R^{2.1} is independently —C(O)NR^{2C}NR^{2A}R^{2B}. In embodiments, R^{2.1} is independently —OR^{2A}. In embodiments, R^{2.1} is independently —NR^{2A}SO₂R^{2B}In embodiments, R^{2.1} is independently —NR^{2A}C(O)R^{2B}. In embodiments, R^{2.1} is independently —NR^{2A}C(O)OR^{2B}. In embodiments, R^{2.1} is independently —NR^{2A}OR^{2B}. In embodiments, R^{2.1} is independently —N₃. In embodiments, R^{2.1} is independently —OH. In embodiments, R^{2.1} is independently —NH₂. In embodiments, R^{2.1} is independently —COOH. In embodiments, R^{2.1} is independently —CONH₂. In embodiments, R^{2.1} is independently —NO₂. In embodiments, R^{2.1} is independently —SH. In embodiments, R^{2.1} is independently halogen. In embodiments, R^{2.1} is independently —F. In embodiments, R^{2.1} is independently —Cl. In embodiments, R^{2.1} is independently —Br. In embodiments, R^{2.1} is independently —I. In embodiments, R^{2.1} is independently —CF₃. In embodiments, R^{2.1} is independently —CHF₂. In embodiments, R^{2.1} is independently —CH₂F. In embodiments, R^{2.1} is independently —OCF₃. In embodiments, R^{2.1} is independently —OCH₂F. In embodiments, R^{2.1} is independently —OCHF₂. In embodiments, R^{2.1} is independently —OCH₃. In embodiments, R^{2.1} is independently —OCH₂CH₃. In embodiments, R^{2.1} is independently —OCH₂CH₂CH₃. In embodiments, R^{2.1} is independently —OCH(CH₃)₂. In embodiments, R^{2.1} is independently —OC(CH₃)₃. In embodiments, R^{2.1} is independently —SCH₃. In embodiments, R^{2.1} is independently —SCH₂CH₃. In embodiments, R^{2.1} is independently —SCH₂CH₂CH₃. In embodiments, R^{2.1} is independently —SCH(CH₃)₂. In embodiments, R^{2.1} is independently —SC(CH₃)₃. In embodiments, R^{2.1} is independently —CH₃. In embodiments, R^{2.1} is independently —CH₂CH₃. In embodiments, R^{2.1} is independently —CHCH₂. In embodiments, R^{2.1} is independently —CH₂CH₂CH₃. In embodiments, R^{2.1} is independently —CH(CH₃)₂. In embodiments, R^{2.1} is independently —C(CH₃)₃.

In embodiments, R^{2.2} is independently hydrogen. In embodiments, R^{2.2} is independently oxo. In embodiments, R^{2.2} is independently halogen. In embodiments, R^{2.2} is independently —CX²₃. In embodiments, R^{2.2} is independently —CHX²₂. In embodiments, R^{2.2} is independently —CH₂X². In embodiments, R^{2.2} is independently —OCX²₃. In embodiments, R^{2.2} is independently —OCH₂X². In embodiments, R^{2.2} is independently —OCHX²₂. In embodiments, R^{2.2} is independently —CN. In embodiments, R^{2.2} is independently —SOₙ₂R^{2A}. In embodiments, R^{2.2} is independently —SOᵥ₂NR^{2A}R^{2B}. In embodiments, R^{2.2} is independently —NR^{2C}C(O)NR^{2A}R^{2B}. In embodiments, R^{2.2} is independently —N(O)ₘ₂. In embodiments, R^{2.2} is independently —NR^{2A}R^{2B}. In embodiments, R^{2.2} is independently —NR^{2C}NR^{2A}R^{2B}. In embodiments, R^{2.2} is independently —C(O)R^{2A}. In embodiments, R^{2.2} is independently —C(O)OR^{2A}. In embodiments, R^{2.2} is independently —C(O)NR^{2A}R^{2B}. In embodiments, R^{2.2} is independently —C(O)NR^{2C}NR^{2A}R^{2B}. In embodiments, R^{2.2} is independently —OR^{2A}. In embodiments, R^{2.2} is independently —NR^{2A}SO₂R^{2B}In embodiments, R^{2.2} is independently —NR^{2A}C(O)R^{2B}. In embodiments, R^{2.2} is independently —NR^{2A}C(O)OR^{2B}. In embodiments, R^{2.2} is independently —NR^{2A}OR^{2B}. In embodiments, R^{2.2} is independently —N₃. In embodiments, R^{2.2} is independently —OH. In embodiments, R^{2.2} is independently —NH₂. In embodiments, R^{2.2} is independently —COOH. In embodiments, R^{2.2} is independently —CONH₂. In embodiments, R^{2.2} is independently —NO₂. In embodiments, R^{2.2} is independently —SH. In embodiments, R^{2.2} is independently halogen. In embodiments, R^{2.2} is independently —F. In embodiments, R^{2.2} is independently —Cl. In embodiments, R^{2.2} is independently —Br. In embodiments, R^{2.2} is independently —I. In embodiments, R^{2.2} is independently —CF₃. In embodiments, R^{2.2} is independently —CHF₂. In embodiments, R^{2.2} is independently —CH₂F. In embodiments, R^{2.2} is independently —OCF₃. In embodiments, R^{2.2} is independently —OCH₂F. In embodiments, R^{2.2} is independently —OCHF₂. In embodiments, R^{2.2} is independently —OCH₃. In embodiments, R^{2.2} is independently —OCH₂CH₃. In embodiments, R^{2.2} is independently —OCH₂CH₂CH₃. In embodiments, R^{2.2} is independently —OCH(CH₃)₂. In embodiments, R^{2.2} is independently —OC(CH₃)₃. In embodiments, R^{2.2} is independently —SCH₃. In embodiments, R^{2.2} is independently —SCH₂CH₃. In embodiments, R^{2.2} is independently —SCH₂CH₂CH₃. In embodiments, R^{2.2} is independently —SCH(CH₃)₂. In embodiments, R^{2.2} is independently —SC(CH₃)₃. In embodiments, R^{2.2} is independently —CH₃. In embodiments, R^{2.2} is independently —CH₂CH₃. In embodiments, R^{2.2} is independently —CHCH₂. In embodiments, R^{2.2} is independently —CH₂CH₂CH₃. In embodiments, R^{2.2} is independently —CH(CH₃)₂. In embodiments, R^{2.2} is independently —C(CH₃)₃.

In embodiments, R^{2.3} is independently hydrogen. In embodiments, R^{2.3} is independently oxo. In embodiments, R^{2.3} is independently halogen. In embodiments, R^{2.3} is independently —CX²₃. In embodiments, R^{2.3} is independently —CHX²₂. In embodiments, R^{2.3} is independently —CH₂X². In embodiments, R^{2.3} is independently —OCX²₃. In embodiments, R^{2.3} is independently —OCH₂X². In embodiments, R^{2.3} is independently —OCHX²₂. In embodiments, R^{2.3} is independently —CN. In embodiments, R^{2.3} is independently —SOₙ₂R^{2A}. In embodiments, R^{2.3} is independently —SOᵥ₂NR^{2A}R^{2B}. In embodiments, R^{2.3} is independently —NR^{2C}C(O)NR^{2A}R^{2B}. In embodiments, R^{2.3} is independently —N(O)ₘ₂. In embodiments, R^{2.3} is independently —NR^{2A}R^{2B}. In embodiments, R^{2.3} is independently —NR^{2C}NR^{2A}R^{2B}. In embodiments, R^{2.3} is independently —C(O)R^{2A}. In embodiments, R^{2.3} is independently —C(O)OR^{2A}. In embodiments, R^{2.3} is independently —C(O)NR^{2A}R^{2B}. In embodiments, R^{2.3} is independently —C(O)NR^{2C}NR^{2A}R^{2B}. In embodiments, R^{2.3} is independently —OR^{2A}. In embodiments, R^{2.3} is independently —NR^{2A}SO₂R^{2B}In embodiments, R^{2.3} is independently —NR^{2A}C(O)R^{2B}. In embodiments, R^{2.3} is independently —NR^{2A}C(O)OR^{2B}. In embodiments, R^{2.3} is independently —NR^{2A}OR^{2B}. In embodiments, R^{2.3} is independently —N₃. In embodiments, R^{2.3} is independently —OH. In embodiments, R^{2.3} is independently —NH₂. In embodiments, R^{2.3} is independently —COOH. In embodiments, R^{2.3} is independently —CONH₂. In embodiments, R^{2.3} is independently —NO₂. In embodiments, R^{2.3} is independently —SH. In embodiments, R^{2.3} is independently halogen. In embodiments, R^{2.3} is independently —F. In embodiments, R^{2.3} is independently —Cl. In embodiments, R^{2.3} is independently —Br. In embodiments, R^{2.3} is independently —I. In embodiments, R^{2.3} is independently —CF₃. In embodiments, R^{2.3} is independently —CHF₂. In embodiments, R^{2.3} is independently —CH₂F. In embodiments, R^{2.3} is independently —OCF₃. In embodiments, R^{2.3} is independently —OCH₂F. In embodiments, R^{2.3} is independently —OCHF₂. In embodiments, R^{2.3} is independently —OCH₃. In embodiments, R^{2.3} is independently —OCH₂CH₃. In embodiments, R^{2.3} is independently —OCH₂CH₂CH₃. In embodiments, R^{2.3} is independently —OCH(CH₃)₂. In embodiments, R^{2.3} is independently —OC(CH₃)₃. In embodiments, R^{2.3} is independently —SCH₃. In embodiments, R^{2.3} is independently —SCH₂CH₃. In embodiments, R^{2.3} is independently —SCH₂CH₂CH₃. In embodiments, R^{2.3} is independently —SCH(CH₃)₂. In embodiments, R^{2.3} is independently —SC(CH₃)₃. In embodiments, R^{2.3} is independently —CH₃. In embodiments, R^{2.3} is independently —CH₂CH₃. In embodiments, R^{2.3} is independently —CHCH₂. In embodiments, R^{2.3} is independently —CH₂CH₂CH₃. In embodiments, R^{2.3} is independently —CH(CH₃)₂. In embodiments, R^{2.3} is independently —C(CH₃)₃.

In embodiments, R^{2.4} is independently hydrogen. In embodiments, R^{2.4} is independently oxo. In embodiments, R^{2.4} is independently halogen. In embodiments, R^{2.4} is independently —CX²₃. In embodiments, R^{2.4} is independently —CHX²₂. In embodiments, R^{2.4} is independently —CH₂X². In embodiments, R^{2.4} is independently —OCX²₃. In embodiments, R^{2.4} is independently —OCH₂X². In embodiments, R^{2.4} is independently —OCHX²₂. In embodiments, R^{2.4} is independently —CN. In embodiments, R^{2.4} is independently —SOₙ₂R^{2A}. In embodiments, R^{2.4} is independently —SOᵥ₂NR^{2A}R^{2B}. In embodiments, R^{2.4} is independently —NR^{2C}C(O)NR^{2A}R^{2B}. In embodiments, R^{2.4} is independently —N(O)ₘ₂. In embodiments, R^{2.4} is independently —NR^{2A}R^{2B}. In embodiments, R^{2.4} is independently —NR^{2C}NR^{2A}R^{2B}. In embodiments, R^{2.4} is independently —C(O)R^{2A}. In embodiments, R^{2.4} is independently —C(O)OR^{2A}. In embodiments, R^{2.4} is independently —C(O)NR^{2A}R^{2B}. In embodiments, R^{2.4} is independently —C(O)NR^{2C}NR^{2A}R^{2B}. In embodiments, R^{2.4} is independently —OR^{2A}. In embodiments, R^{2.4} is independently —NR^{2A}SO₂R^{2B}In embodiments, R^{2.4} is independently —NR^{2A}C(O)R^{2B}. In embodiments, R^{2.4} is independently —NR^{2A}C(O)OR^{2B}. In embodiments, R^{2.4} is independently —NR^{2A}OR^{2B}. In embodiments, R^{2.4} is independently —N₃. In embodiments, R^{2.4} is independently —OH. In embodiments, R^{2.4} is independently —NH₂. In embodiments, R^{2.4} is independently —COOH. In embodiments, R^{2.4} is independently —CONH₂. In embodiments, R^{2.4} is independently —NO₂. In embodiments, R^{2.4} is independently —SH. In embodiments, R^{2.4} is independently halogen. In embodiments, R^{2.4} is independently —F. In embodiments, R^{2.4} is independently —Cl. In embodiments, R^{2.4} is independently —Br. In embodiments, R^{2.4} is independently —I. In embodiments, R^{2.4} is independently —CF₃. In embodiments, R^{2.4} is independently —CHF₂. In embodiments, R^{2.4} is independently —CH₂F. In embodiments, R^{2.4} is independently —OCF₃. In embodiments, R^{2.4} is independently —OCH₂F. In embodiments, R^{2.4} is independently —OCHF₂. In embodiments, R^{2.4} is independently —OCH₃. In embodiments, R^{2.4} is independently —OCH₂CH₃. In embodiments, R^{2.4} is independently —OCH₂CH₂CH₃. In embodiments, R^{2.4} is independently —OCH(CH₃)₂. In embodiments, R^{2.4} is independently —OC(CH₃)₃. In embodiments, R^{2.4} is independently —SCH₃. In embodiments, R^{2.4} is independently —SCH₂CH₃. In embodiments, R^{2.4} is independently —SCH₂CH₂CH₃. In embodiments, R²⁴ is independently —SCH(CH₃)₂. In embodiments, R^{2.4} is independently —SC(CH₃)₃. In embodiments, R^{2.4} is independently —CH₃. In embodiments, R^{2.4} is independently —CH₂CH₃. In embodiments, R^{2.4} is independently —CHCH₂. In embodiments, R^{2.4} is independently —CH₂CH₂CH₃. In embodiments, R^{2.4} is independently —CH(CH₃)₂. In embodiments, R^{2.4} is independently —C(CH₃)₃.

In embodiments, R^{2.5} is independently hydrogen. In embodiments, R^{2.5} is independently oxo. In embodiments, R^{2.5} is independently halogen. In embodiments, R^{2.5} is independently —CX²₃. In embodiments, R^{2.5} is independently —CHX²₂. In embodiments, R^{2.5} is independently —CH₂X². In embodiments, R^{2.5} is independently —OCX²₃. In embodiments, R^{2.5} is independently —OCH₂X². In embodiments, R^{2.5} is independently —OCHX²₂. In embodiments, R^{2.5} is independently —CN. In embodiments, R^{2.5} is independently —SOₙ₂R^{2A}. In embodiments, R^{2.5} is independently —SOᵥ₂NR^{2A}R^{2B}. In embodiments, R^{2.5} is independently —NR^{2C}C(O)NR^{2A}R^{2B}. In embodiments, R^{2.5} is independently —N(O)ₘ₂. In embodiments, R^{2.5} is independently —NR^{2A}R^{2B}. In embodiments, R^{2.5} is independently —NR^{2C}NR^{2A}R^{2B}. In embodiments, R^{2.5} is independently —C(O)R^{2A}. In embodiments, R^{2.5} is independently —C(O)OR^{2A}. In embodiments, R^{2.5} is independently —C(O)NR^{2A}R^{2B}. In embodiments, R^{2.5} is independently —C(O)NR^{2C}NR^{2A}R^{2B}. In embodiments, R^{2.5} is independently —OR^{2A}. In embodiments, R^{2.5} is independently —NR^{2A}SO₂R^{2B}In embodiments, R^{2.5} is independently —NR^{2A}C(O)R^{2B}. In embodiments, R^{2.5} is independently —NR^{2A}C(O)OR^{2B}. In embodiments, R^{2.5} is independently —NR^{2A}OR^{2B}. In embodiments, R^{2.5} is independently —N₃. In embodiments, R^{2.5} is independently —OH. In embodiments, R^{2.5} is independently —NH₂. In embodiments, R^{2.5} is independently —COOH. In embodiments, R^{2.5} is independently —CONH₂. In embodiments, R^{2.5} is independently —NO₂. In embodiments, R^{2.5} is independently —SH. In embodiments, R^{2.5} is independently halogen. In embodiments, R^{2.5} is independently —F. In embodiments, R^{2.5} is independently —Cl. In embodiments, R^{2.5} is independently —Br. In embodiments, R^{2.5} is independently —I. In embodiments, R^{2.5} is independently —CF₃. In embodiments, R^{2.5} is independently —CHF₂. In embodiments, R^{2.5} is independently —CH₂F. In embodiments, R^{2.5} is independently —OCF₃. In embodiments, R^{2.5} is independently —OCH₂F. In embodiments, R^{2.5} is independently —OCHF₂. In embodiments, R^{2.5} is independently —OCH₃. In embodiments, R^{2.5} is independently —OCH₂CH₃. In embodiments, R^{2.5} is independently —OCH₂CH₂CH₃. In embodiments, R^{2.5} is independently —OCH(CH₃)₂. In embodiments, R^{2.5} is independently —OC(CH₃)₃. In embodiments, R^{2.5} is independently —SCH₃. In embodiments, R²⁵ is independently —SCH₂CH₃. In embodiments, R²⁵ is independently —SCH₂CH₂CH₃. In embodiments, R^{2.5} is independently —SCH(CH₃)₂. In embodiments, R^{2.5} is independently —SC(CH₃)₃. In embodiments, R^{2.5} is independently —CH₃. In embodiments, R^{2.5} is independently —CH₂CH₃. In embodiments, R^{2.5} is independently —CHCH₂. In embodiments, R^{2.5} is independently —CH₂CH₂CH₃. In embodiments, R^{2.5} is independently —CH(CH₃)₂. In embodiments, R^{2.5} is independently —C(CH₃)₃.

In embodiments, unless otherwise indicated, a compound described herein is a racemic mixture of all stereoisomers. In embodiments, unless otherwise indicated, a compound described herein is a racemic mixture of all enantiomers. In embodiments, unless otherwise indicated, a compound described herein is a racemic mixture of two opposite stereoisomers. In embodiments, unless otherwise indicated, a compound described herein is a racemic mixture of two opposite enantiomers. In embodiments, unless otherwise indicated, a compound described herein is a single stereoisomer. In embodiments, unless otherwise indicated, a compound described herein is a single enantiomer. In embodiments, the compound is a compound described herein (e.g., in an aspect, embodiment, example, figure, table, scheme, or claim).

In embodiments, the compound contacts one or more amino acids corresponding to S280, Y314, Y464, F318, H440, F273, F351, I272, V332, I339, or L344 of human peroxisome proliferator-activated receptor alpha. In embodiments, the compound contacts an amino acid corresponding to S280 in human peroxisome proliferator-activated receptor alpha. In embodiments, the compound contacts an amino acids corresponding to Y314 of human peroxisome proliferator-activated receptor alpha. In embodiments, the compound contacts an amino acids corresponding to Y464 of human peroxisome proliferator-activated receptor alpha. In embodiments, the compound contacts an amino acids corresponding to F318 of human peroxisome proliferator-activated receptor alpha. In embodiments, the compound contacts an amino acids corresponding to H440 of human peroxisome proliferator-activated receptor alpha. In embodiments, the compound contacts an amino acids corresponding to F273 of human peroxisome proliferator-activated receptor alpha. In embodiments, the compound contacts an amino acids corresponding to F351 of human peroxisome proliferator-activated receptor alpha. In embodiments, the compound contacts an amino acids corresponding to I339 of human peroxisome proliferator-activated receptor alpha. In embodiments, the compound contacts an amino acids corresponding to C275 of human peroxisome proliferator-activated receptor alpha. In embodiments, the compound contacts an amino acids corresponding to C276 of human peroxisome proliferator-activated receptor alpha. In embodiments, the compound contacts an amino acids corresponding to V332 of human peroxisome proliferator-activated receptor alpha. In embodiments, the compound contacts an amino acids corresponding to E251 of human peroxisome proliferator-activated receptor alpha. In embodiments, the compound contacts an amino acids corresponding to I272 of human peroxisome proliferator-activated receptor alpha. In embodiments, the compound contacts an amino acids corresponding to V332 of human peroxisome proliferator-activated receptor alpha. In embodiments, the compound contacts an amino acids corresponding to I339 of human peroxisome proliferator-activated receptor alpha. In embodiments, the compound contacts an amino acids corresponding to L344 of human peroxisome proliferator-activated receptor alpha.

In embodiments, the compound is not a compound described in WO 2002/038553 or WO 2011/163594.

In embodiments, the compound is not:

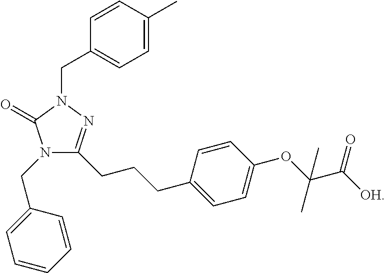 [SMILES: Cc1ccc(Cn2nc(CCCc3ccc(OC(C)(C)C(=O)O)cc3)n(Cc3ccccc3)c2=O)cc1]

In embodiments, the compound is not:

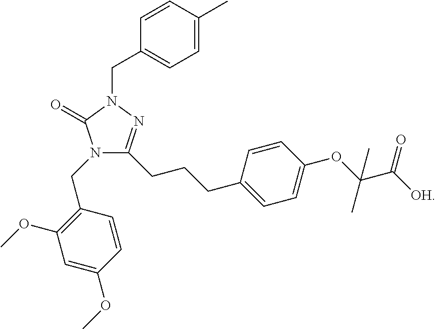 [SMILES: COc1ccc(Cn2c(CCCc3ccc(OC(C)(C)C(=O)O)cc3)nn(Cc3ccc(C)cc3)c2=O)c(OC)c1]

In embodiments, the compound is not:

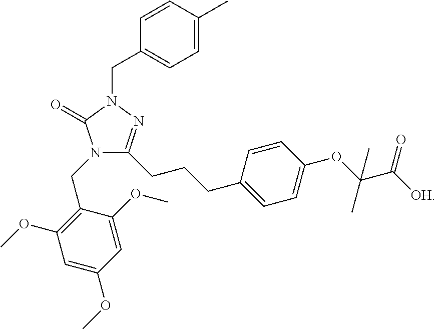 [SMILES: COc1cc(OC)c(Cn2c(CCCc3ccc(OC(C)(C)C(=O)O)cc3)nn(Cc3ccc(C)cc3)c2=O)c(OC)c1]

In embodiments, the compound is not:

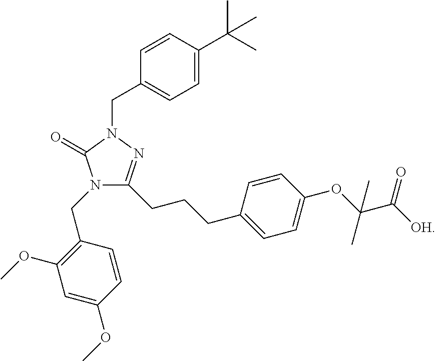 [SMILES: COc1ccc(Cn2c(CCCc3ccc(OC(C)(C)C(=O)O)cc3)nn(Cc3ccc(C(C)(C)C)cc3)c2=O)c(OC)c1]

In embodiments, the compound is not:

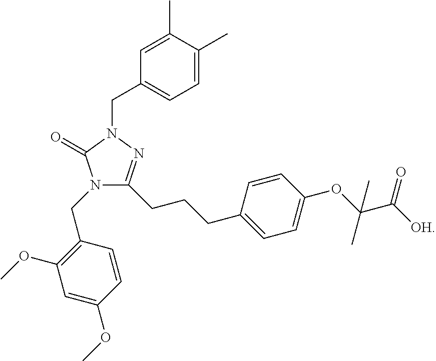 [SMILES: COc1ccc(Cn2c(CCCc3ccc(OC(C)(C)C(=O)O)cc3)nn(Cc3ccc(C)c(C)c3)c2=O)c(OC)c1]

In embodiments, the compound is not:

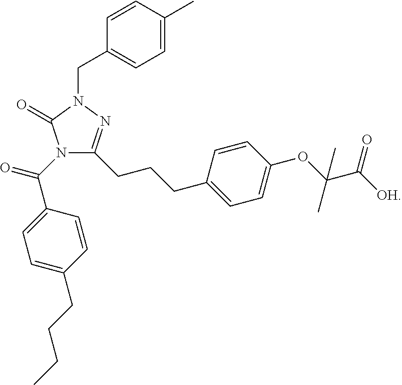 [SMILES: CCCCc1ccc(C(=O)n2c(CCCc3ccc(OC(C)(C)C(=O)O)cc3)nn(Cc3ccc(C)cc3)c2=O)cc1]

In embodiments, the compound is not:

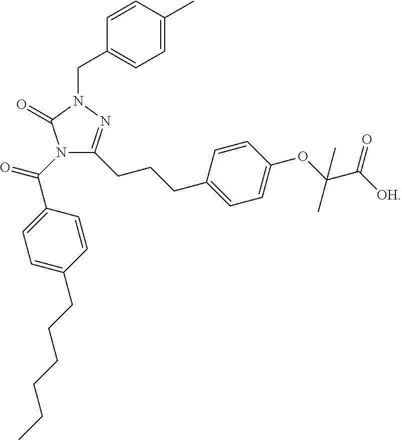 [SMILES: CCCCCCc1ccc(C(=O)n2c(CCCc3ccc(OC(C)(C)C(=O)O)cc3)nn(Cc3ccc(C)cc3)c2=O)cc1]

In embodiments, the compound is not:

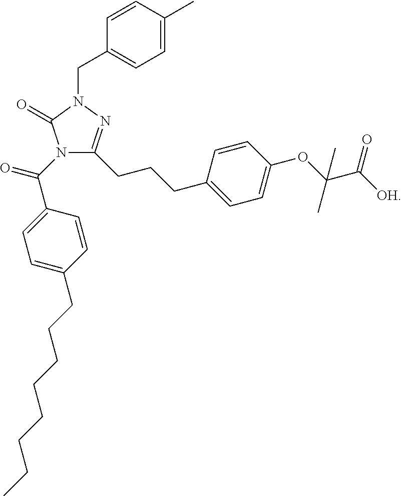 [SMILES: CCCCCCCCc1ccc(C(=O)n2c(CCCc3ccc(OC(C)(C)C(=O)O)cc3)nn(Cc3ccc(C)cc3)c2=O)cc1]

### Pharmaceutical Compositions

In an aspect is provided a pharmaceutical composition including a compound described herein (e.g., a compound of formula I, Ia, II, IIa, IIb, IIc, IId, or IIe; or an embodiment thereof), or pharmaceutically acceptable salt thereof, and a pharmaceutically acceptable excipient.

In embodiments of the pharmaceutical compositions, the compound, or pharmaceutically acceptable salt thereof, is included in a therapeutically effective amount.

### III. Methods of Treatment

In an aspect is provided a method of treating non-alcoholic fatty liver disease (NAFLD), the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating non-alcoholic steatohepatitis (NASH), the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating a disease associated with peroxisome proliferator-activated receptor activity including administering to a subject in need thereof an effective amount of a compound described herein (e.g., a compound of formula I, Ia, II, IIa, IIb, IIc, IId, or IIe; or an embodiment thereof). In embodiments, the disease is associated with peroxisome proliferator-activated receptor α activity.

In an aspect is provided a method of treating obesity, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating a hormone disorder, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating dyslipidemia, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating a lipid disorder, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount. In embodiments, the lipid disorder is dyslipidemia. In embodiments, the lipid disorder is hyperlipidemia. In embodiments, the lipid disorder is hypertriglyceridemia. In embodiments, the lipid disorder is hypercholesterolemia. In embodiments, the lipid disorder is hyperlipoproteinemia. In embodiments, the lipid disorder is combined hyperlipidemia. In embodiments, the lipid disorder is hyperchylomicronemia (e.g., familial). In embodiments, the lipid disorder is familial apoprotein CII deficiency. In embodiments, the lipid disorder is familial hypercholesterolemia. In embodiments, the lipid disorder is familial combined hyperlipidemia. In embodiments, the lipid disorder is familial dysbetalipoproteinemia. In embodiments, the lipid disorder is familial hypertriglyceridemia.

In an aspect is provided a method of treating a metabolic disorder, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating syndrome X (metabolic syndrome), the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating diabetes, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating type 1 diabetes, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating type 2 diabetes, the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of treating fibrosis (e.g., liver fibrosis), the method including administering to a subject in need thereof an effective amount of a compound described herein (e.g., a compound of formula I, Ia, II, IIa, IIb, IIc, IId, or IIe; or an embodiment thereof). In embodiments, the compound is included in a therapeutically effective amount.

In an aspect is provided a method of modulating the level of a lipid in a subject (e.g., compared to a control, such as absence of the method), the method including administering to a subject in need thereof an effective amount of a compound described herein (e.g., a compound of formula I, Ia, II, IIa, IIb, IIc, IId, or IIe; or an embodiment thereof). (e.g., a compound of formula I, Ia, II, IIa, IIb, IIc, IId, or IIe; or an embodiment thereof). In embodiments, the method modulates (e.g., decreases) the level of chylomicrons in a subject. In embodiments, the method modulates (e.g., decreases) the level of triglycerides in a subject (e.g., to less than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, or 1000 mg/dL). In embodiments, the method modulates (e.g., decreases) the level of phospholipids in a subject. In embodiments, the method modulates (e.g., decreases) the level of cholesterol in a subject (e.g., to less than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, or 1000 mg/dL). In embodiments, the method modulates (e.g., decreases) the level of lipoproteins in a subject. In embodiments, the method modulates (e.g., decreases) the level ofvery-low-density lipoproteins (VLDL) in a subject. In embodiments, the method modulates (e.g., decreases) the level of intermediate-density lipoproteins (IDL) in a subject. In embodiments, the method modulates (e.g., decreases) the level of low-density lipoproteins (LDL) in a subject (e.g., to less than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 350, 400, 450, 500, 550, 600, 650, 700, 750, 800, 850, 900, 950, or 1000 mg/dL). In embodiments, the method modulates (e.g., increases) the level of high-density lipoproteins (HDL) in a subject (e.g., to greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mg/dL).

### IV. Methods of Activation

In an aspect is provided a method of increasing peroxisome proliferator-activated receptor activity, the method including contacting the peroxisome proliferator-activated receptor with a compound described herein (e.g., a compound of formula I, Ia, II, IIa, IIb, IIc, IId, or IIe; or an embodiment thereof). In embodiments, the peroxisome proliferator-activated receptor is peroxisome proliferator-activated receptor α.

In embodiments, the compound increases peroxisome proliferator-activated receptor α (PPAR α) at least 2-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 5-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 10-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 20-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 50-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 100-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 500-fold more potently than the compound increases a different peroxisome proliferator-activated receptor (e.g., PPAR β, PPAR δ, and/or PPAR γ). In embodiments, the compound increases peroxisome proliferator-activated receptor α (PPAR α) at least 1000-fold more potently than the compound increases a different peroxisome proliferator-activated receptor (e.g., PPAR β, PPAR δ, and/or PPAR γ).

In embodiments, the peroxisome proliferator-activated receptor α is a human peroxisome proliferator-activated receptor α. In embodiments, the compound contacts one or more amino acids corresponding to S280, Y314, Y464, F318, H440, F273, F351, I272, V332, I339, or L344, of human peroxisome proliferator-activated receptor alpha. In embodiments, the compound contacts an amino acid corresponding to S280 in human peroxisome proliferator-activated receptor alpha. In embodiments, the compound contacts an amino acids corresponding to Y314 of human peroxisome proliferator-activated receptor alpha. In embodiments, the compound contacts an amino acids corresponding to Y464 of human peroxisome proliferator-activated receptor alpha. In embodiments, the compound contacts an amino acids corresponding to F318 of human peroxisome proliferator-activated receptor alpha. In embodiments, the compound contacts an amino acids corresponding to H440 of human peroxisome proliferator-activated receptor alpha. In embodiments, the compound contacts an amino acids corresponding to F273 of human peroxisome proliferator-activated receptor alpha. In embodiments, the compound contacts an amino acids corresponding to F351 of human peroxisome proliferator-activated receptor alpha. In embodiments, the compound contacts an amino acids corresponding to I339 of human peroxisome proliferator-activated receptor alpha. In embodiments, the compound contacts an amino acids corresponding to C275 of human peroxisome proliferator-activated receptor alpha. In embodiments, the compound contacts an amino acids corresponding to C276 of human peroxisome proliferator-activated receptor alpha. In embodiments, the compound contacts an amino acids corresponding to V332 of human peroxisome proliferator-activated receptor alpha. In embodiments, the compound contacts an amino acids corresponding to E251 of human peroxisome proliferator-activated receptor alpha. In embodiments, the compound contacts an amino acids corresponding to I272 of human peroxisome proliferator-activated receptor alpha. In embodiments, the compound contacts an amino acids corresponding to V332 of human peroxisome proliferator-activated receptor alpha. In embodiments, the compound contacts an amino acids corresponding to I339 of human peroxisome proliferator-activated receptor alpha. In embodiments, the compound contacts an amino acids corresponding to L344 of human peroxisome proliferator-activated receptor alpha.

### V. Embodiments

### Embodiment P1

A compound having the formula:

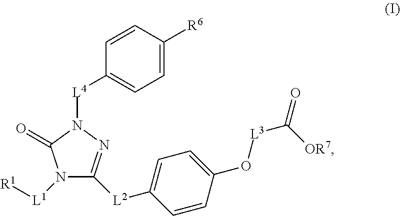

wherein, L¹ is a bond, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L² is unsubstituted C₁-C₆ alkylene; L³ is substituted or unsubstituted C₁-C₆ alkylene; L⁴ is unsubstituted C₁-C₄ alkylene; R¹ is substituted or unsubstituted cycloalkyl, substituted or unsubstituted heterocycloalkyl, substituted or unsubstituted aryl, or substituted or unsubstituted heteroaryl; R⁶ is unsubstituted C₁-C₄ alkyl; and R⁷ is hydrogen or, together with the oxygen to which it is attached, forms a prodrug moiety.

### Embodiment P2

The compound of embodiment P1, having the formula:

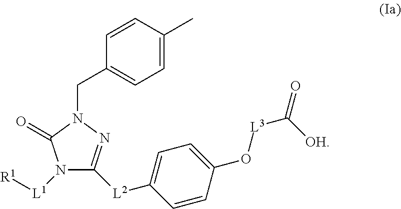

### Embodiment P3

The compound of one of embodiments P1 to P2, wherein L³ is substituted or unsubstituted C₁-C₃ alkylene.

### Embodiment P4

The compound of one of embodiments P1 to P2, wherein L³ is substituted or unsubstituted C₁-C₃ alkylene.

### Embodiment P5

The compound of one of embodiments P1 to P2, wherein L³ is —C(CH₃)₂—.

### Embodiment P6

The compound of one of embodiments P1 to P5, wherein L² is unsubstituted C₂-C₄ alkylene.

### Embodiment P7

The compound of one of embodiments P1 to P5, wherein L² is unsubstituted n-propylene.

### Embodiment P8

The compound of one of embodiments P1 to P7, wherein L¹ is a bond or substituted or unsubstituted alkylene.

### Embodiment P9

The compound of one of embodiments P1 to P7, wherein L¹ is a bond or unsubstituted C₁-C₃ alkylene.

### Embodiment P10

The compound of one of embodiments P1 to P7, wherein L¹ is an unsubstituted C₁-C₃ alkylene.

### Embodiment P11

The compound of one of embodiments P1 to P7, wherein L¹ is an unsubstituted methylene.

### Embodiment P12

The compound of one of embodiments P1 to P11, wherein R¹ is substituted or unsubstituted C₄-C₆ cycloalkyl, substituted or unsubstituted 4 to 6 membered heterocycloalkyl, substituted or unsubstituted phenyl, or substituted or unsubstituted 5 to 6 membered heteroaryl.

### Embodiment P13

The compound of one of embodiments P1 to P11, wherein R¹ is substituted or unsubstituted phenyl or substituted or unsubstituted 5 to 6 membered heteroaryl.

### Embodiment P14

The compound of one of embodiments 1 to 11, wherein R¹ is substituted or unsubstituted phenyl.

### Embodiment P15

The compound of one of embodiments P1 to P11, wherein R¹ is R²-substituted or unsubstituted phenyl or R²-substituted or unsubstituted 5 to 6 membered heteroaryl; R² is independently halogen, —CX²₃, —CHX²₂, —CH₂X², —OCX²₃, —OCH₂X², —OCHX²₂, —CN, —SOₙ₂R^{2A}, —SOᵥ₂NR^{2A}R^{2B}, —NR^{2C}C(O)NR^{2A}R^{2B}, —N(O)ₘ₂, —NR^{2A}R^{2B}, —NR² CNR^{2A}R^{2B}, —C(O)R^{2A}, —C(O)OR^{2A}, —C(O)NR^{2A}R^{2B}, —C(O)NR^{2C}NR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A}, R^{2B}, and R^{2C} are independently hydrogen, —CX₃, —CHX₂, —CH₂X, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; m2 is independently 1 or 2; v2 is independently 1 or 2; n2 is independently an integer from 0 to 4; and X and X² are independently —Cl, —Br, —I or —F.

### Embodiment P16

The compound of one of embodiments P1 to P15, having the formula:

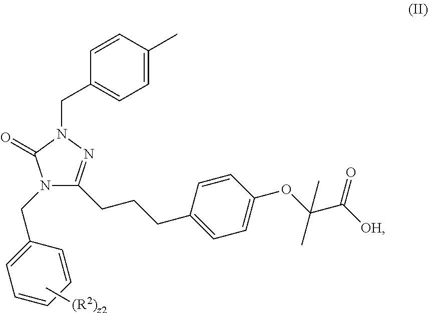

wherein z2 is an integer from 0 to 5.

### Embodiment P17

The compound of embodiment P16, wherein z2 is 1.

### Embodiment P18

The compound of embodiment P16, wherein z2 is 2.

### Embodiment P19

The compound of embodiment P16, wherein z2 is 3.

### Embodiment P20

The compound of one of embodiments P1 to P15, having the formula:

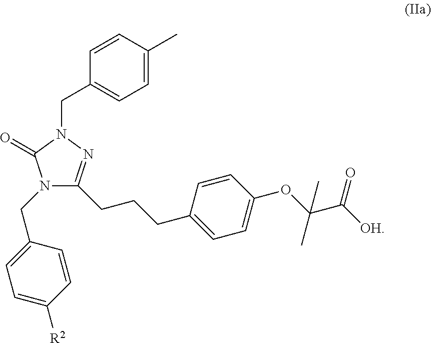

### Embodiment P21

The compound of one of embodiments P1 to P15, having the formula:

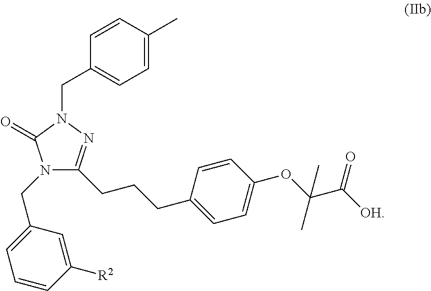

### Embodiment P22

The compound of one of embodiments P1 to P15, having the formula:

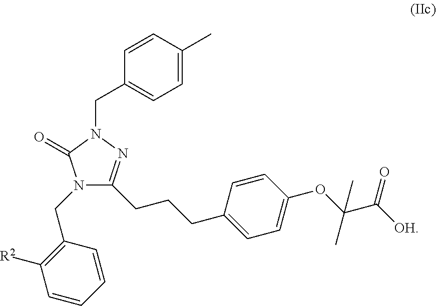

### Embodiment P23

The compound of one of embodiments P1 to P15, having the formula:

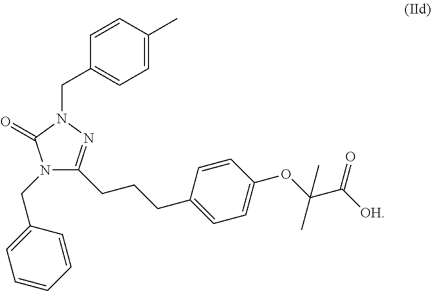 [SMILES: Cc1ccc(Cn2nc(CCCc3ccc(OC(C)(C)C(=O)O)cc3)n(Cc3ccccc3)c2=O)cc1]

### Embodiment P24

The compound of one of embodiments P1 to P22, wherein R² is independently halogen, —CX²₃, —CHX²₂, —CH₂X², —OCX²₃, —OCH₂X², —OCHX²₂, —OH, substituted or unsubstituted C₁-C₄ alkyl, or substituted or unsubstituted 2 to 4 membered heteroalkyl; and X² is independently —Cl, —Br, —I or —F.

### Embodiment P25

The compound of one of embodiments P1 to P22, wherein R² is independently halogen, —OCH₃, —OH, or unsubstituted C₁-C₄ alkyl.

### Embodiment P26

The compound of embodiment P1, having the formula:

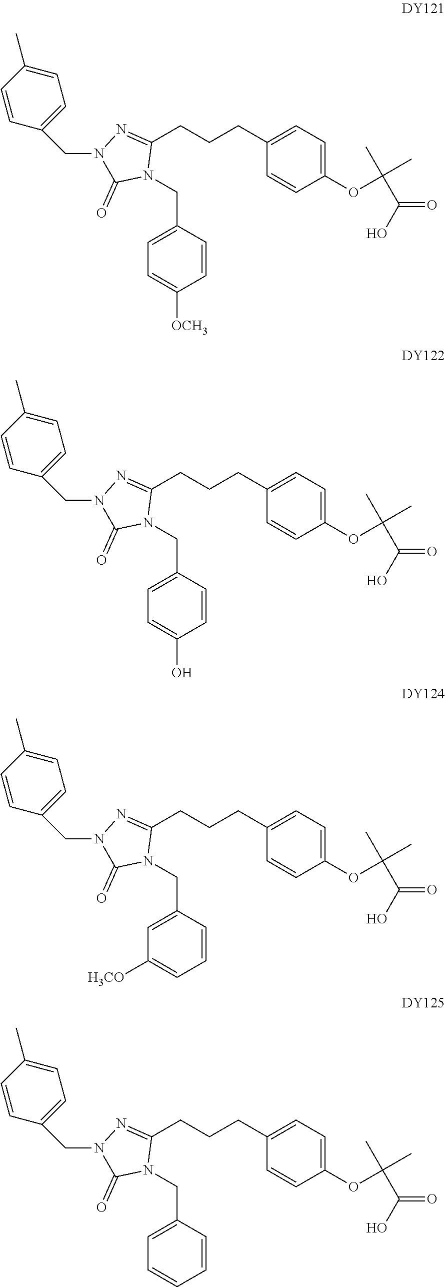 [SMILES: COc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(O)cc3)c2=O)cc1 ; COc1cccc(Cn2c(CCCc3ccc(OC(C)(C)C(=O)O)cc3)nn(Cc3ccc(C)cc3)c2=O)c1 ; Cc1ccc(Cn2nc(CCCc3ccc(OC(C)(C)C(=O)O)cc3)n(Cc3ccccc3)c2=O)cc1]

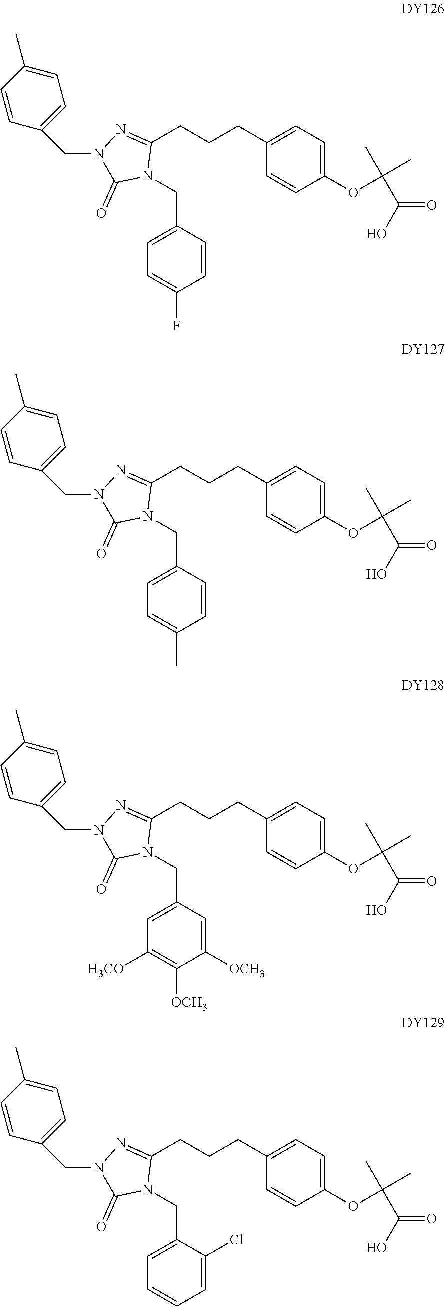 [SMILES: Cc1ccc(Cn2nc(CCCc3ccc(OC(C)(C)C(=O)O)cc3)n(Cc3ccc(F)cc3)c2=O)cc1 ; Cc1ccc(Cn2nc(CCCc3ccc(OC(C)(C)C(=O)O)cc3)n(Cc3ccc(C)cc3)c2=O)cc1 ; COc1cc(Cn2c(CCCc3ccc(OC(C)(C)C(=O)O)cc3)nn(Cc3ccc(C)cc3)c2=O)cc(OC)c1OC ; Cc1ccc(Cn2nc(CCCc3ccc(OC(C)(C)C(=O)O)cc3)n(Cc3ccccc3Cl)c2=O)cc1]

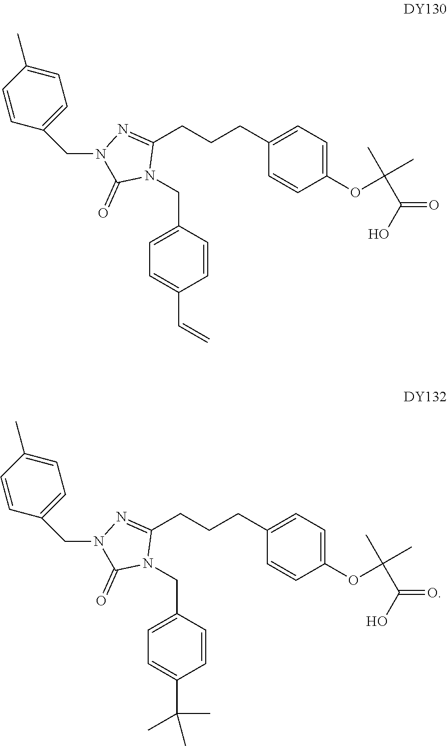 [SMILES: C=Cc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(C(C)(C)C)cc3)c2=O)cc1]

### Embodiment P27

The compound of embodiment P1, having the formula:

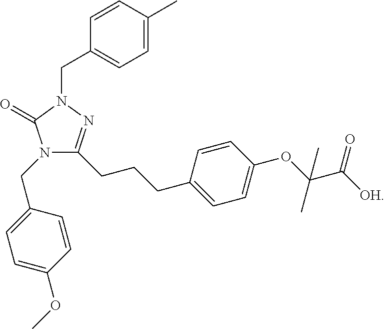 [SMILES: COc1ccc(Cn2c(CCCc3ccc(OC(C)(C)C(=O)O)cc3)nn(Cc3ccc(C)cc3)c2=O)cc1]

### Embodiment P28

A pharmaceutical composition comprising the compound of any one of embodiments P1 to P27 and a pharmaceutically acceptable excipient.

### Embodiment P29

A method of increasing a peroxisome proliferator-activated receptor activity, said method comprising contacting the peroxisome proliferator-activated receptor with the compound of one of embodiments P1 to P27.

### Embodiment P30

The method of embodiment P29, wherein the peroxisome proliferator-activated receptor is peroxisome proliferator-activated receptor α.

### Embodiment P31

A method of treating non-alcoholic fatty liver disease in a subject in need thereof, the method comprising administering to the subject a compound of one of embodiments P1 to P27.

### Embodiment P32

A method of treating non-alcoholic steatohepatitis in a subject in need thereof, the method comprising administering to the subject a compound of one of embodiments P1 to P27.

### Embodiment P33

A method of treating obesity in a subject in need thereof, the method comprising administering to the subject a compound of one of embodiments P1 to P27.

### Embodiment P34

A method of treating diabetes in a subject in need thereof, the method comprising administering to the subject a compound of one of embodiments P1 to P27.

### Embodiment P35

A method of treating metabolic syndrome in a subject in need thereof, the method comprising administering to the subject a compound of one of embodiments P1 to P27.

### Embodiment P36

A method of treating a lipid disorder in a subject in need thereof, the method comprising administering to the subject a compound of one of embodiments P1 to P27.

### Embodiment P37

The method of embodiment P36, wherein the lipid disorder is dyslipidemia, hyperlipidemia, hypertriglyceridemia, hypercholesterolemia, hyperlipoproteinemia, combined hyperlipidemia, hyperchylomicronemia, familial hyperchylomicronemia, familial apoprotein CII deficiency, familial hypercholesterolemia, familial combined hyperlipidemia, familial dysbetalipoproteinemia, or familial hypertriglyceridemia.

### Embodiment P38

A method of modulating the level of a lipid in a subject in need thereof, the method comprising administering to the subject a compound of one of embodiments P1 to P27.

### Embodiment P39

The method of embodiment P38, wherein the method modulates the level of chylomicrons, triglycerides, phospholipids, cholesterol, lipoproteins, very-low-density lipoproteins, intermediate-density lipoproteins, low-density lipoproteins, or high-density lipoproteins.

### Embodiment P40

The method of embodiment P38, wherein the method decreases the level of chylomicrons, triglycerides, phospholipids, cholesterol, lipoproteins, very-low-density lipoproteins, intermediate-density lipoproteins, or low-density lipoproteins.

### Embodiment P41

The method of embodiment P38, wherein the method increases the level of high-density lipoproteins.

### EXAMPLES

PPARα is highly expressed in the liver and PPARα serves as the master regulator of lipid metabolism in liver, especially during fasting. A number of studies in PPARA mRNA expression in subjects with NAFLD have been confirmed. The functional relevance of the changes in PPARα expression was further assessed, and the expression of CPT1A, FGF21 and PDK4, all well-known PPRE-driven PPARα target genes were studied.⁵

The expression of these target genes correlates with changes in PPARα expression, consistently increasing in relation with NASH disappearance, whereas the persistence in NASH associates with unchanged expression of these target genes. These data support the functional relevance of the observed changes in PPARα gene expression.

PPARα ligands are active in liver-derived cells and display the gene expression patterns observed in hepatocytes. A number of human PPARα target genes have been identified via studies in human primary hepatocytes by using specific synthetic agonists to activate PPARα. For example, treatment of human primary hepatocytes cultures treated with PPARα agonists resulted in upregulating gene expression involved in lipid metabolism, thus clearly highlight the key role of PPARα as master regulator of hepatic lipid metabolism. Similarly, downregulating expression of genes is also able to observe via a process known as transrepression. Activation of PPARα leads to a reduction in the basal and/or cytokine-induced expression of acute phase genes such as C-reactive protein (CRP) and fibrinogen (FGB).⁵ These observations have led to the conclusion that the anti-inflammatory effects of PPARα might be attributable to the truncated form of PPARα via interference with the NF-kB pathway, which provide strong evidence that PPARα exerts a major anti-inflammatory action in human liver.

The ability to recruit a wide array of functionally distinct coregulators provides each receptor with the potential to regulate a single gene in different ways depending on the relative concentration and posttranslational status of each coregulator in the cell. This has led to the concept of NR ligands with tissue- or gene-selective activities, i.e. selective NR modulators. Although demonstrated for many NRs, the estrogen receptor (ER) provides perhaps the best studied example of this paradigm (Cheskis et al., 2007). Crystal structures of ER bound to distinct ligands such as estradiol and 4-hydroxy-tamoxifen exhibit distinct conformational changes in the ER ligand binding domain. A consequence of these ligand-specific conformations is that different ligands can promote differential affinities for the numerous coregulators that are present in any given cell. The net result of these distinct molecular signatures is that different ligands can have distinct transcriptional activities on different genes or in different tissues (Shang and Brown, 2002). In effect, different coregulator peptides can be used as “conformation probes” to sort ligands into those with distinct classes of downstream target genes. This has important therapeutic implications as it offers the possibility of identifying synthetic NR ligands that display therapeutic activity without known side-effects. In fact, this is the basis for the clinical success of selective ER modulators (SERMs) tamoxifen and raloxifene.

In recent years, several new molecules that target PPARα, PPARγ, and PPARδ pathways related to NAFLD/NASH pathogenesis, such as liver metabolic homeostasis, inflammation, oxidative stress and fibrosis, have been developed. Insulin resistance with an increased released of free fatty acids, oxidative stress and activation of inflammatory cytokines seem to be key features for transition from NAFLD to NASH and progression of fibrosis. For those with NAFLD/NASH with diabetes, PPARγ based drugs called thiazolidinedione (TZDs) can lessen problems with insulin resistance, making patients more responsive to the insulin they produced, and reduce the need for injections. TZDs often work in cases where other treatments have failed. But they also cause unwanted edema (swelling) and weight gain, which can lead to congestive heart failure. Therefore, insulin sensitizers, such as thiazolidinediones, as well as antioxidant agents, such as vitamin E, even though have been extensively evaluated in phase III clinical trials as options for NASH treatment, however, the trouble is, these drugs act at such a basic level of biology that they have other undesirable impacts.⁶

As compared with PPARγ, PPARα is highly expressed in the liver and regulates the expression of genes encoding lipid and lipoprotein metabolism. PPARα has key roles in regulation of fatty-acid transport as well as peroxisomal and mitochondrial β-oxidation in the liver.⁷ PPARα knockout mice increase the susceptibility to liver steatosis, inflammation, and hepatocellular carcinoma.⁸ Therefore, it has been suggested that PPARα has a protective role against NAFLD pathogenesis. PPARα agonists have been shown to improve the pathologic condition of NAFLD in various preclinical models.⁹

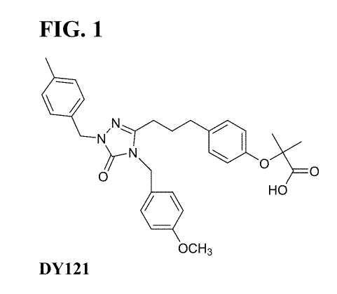

PPARα agonist fenofibric acid (FIG. 1) has been shown to improve the pathologic condition of NAFLD in various preclinical models, indicating a link between NAFLD/NASH and PPARα. However, fenofibric acid can also be associated with an increased risk of unwanted side effects and its efficacy is limited.

To reduce side effects, a new generation of highly potent and selective PPARα modulators (SPPARMα) is being developed that separate the benefits of the PPARα agonists from their unwanted side effects. Among these, aleglitazar (a dual PPARα/γ agonist) and GFT505 (a dual PPAR α/δ agonist) have recently entered late-phase development. Although both compounds are more potent PPARα-activators than fenofibrate in vitro, only aleglitezar is more effective in lowering triglycerides and raising high-density lipoprotein-cholesterol (HDL-C) in humans. However, it is also associated with a potential risk of adverse effects. Indeed, there is a different potential, the chance to develop powerful new PPAR medicines without such risks. The potential is to develop ideal PPARα agonists involving a series of in vitro and in vivo assays to identify the most potent molecules that differentially induce receptor-mediated beneficial effects in specific tissues whilst avoiding unwanted side effects. A potent and efficacious PPARα agonist with an excellent safety profile may provide an opportunity for the treatment of atherosclerosis and dyslipidemia as well as NASH (non-alcoholic steatohepatitis) with minimized side effects that is called “selective PPARα modulators” (SPPARMα).¹⁰ They maximize the beneficial effects and minimize the adverse effects of fibrates. This will provide a quicker, more rational approach to finding better drugs for potent and selective activators that modulate human PPARα more effectively and treat NAFLD/NASH without unwanted side effect.

Thus in addition to the tissue- and gene selective modulators, the potential exists to identify selective PPARα modulators. This inherent flexibility in NR modulation can be exploited to promote a desired therapeutic response while minimizing unrelated or potentially toxic responses. Selective PPARα modulators (SPPARMα) that act as targeted metabolic therapies for NAFLD/NASH can be identified. Previously, a number of PPARγ-agonists with SPPARM properties (e.g. INT131 and MK0533) have been developed for the treatment of T2D. Potential identification of selective PPARα modulators not only are potential new drug candidates but also provide tools to become superior therapeutics for the treatment of liver risk associated with NAFLD/NASH. Thus, for the pharmacological basis, the selective PPARα modulators (SPPARMs) is the development of ideal PPARα-agonists involves a series of in vitro and in vivo assays to identify the most potent molecules that differentially induce receptor-mediated beneficial effects in specific tissues whilst avoiding unwanted side effects.

Obesity and insulin resistance are important risk factors for NAFLD/NASH. Insulin resistance is probably the main driver of NAFLD but also has an important role in the initiation and perpetuation of NASH, including fibrosis progression. Insulin resistance is known to play a critical role in further impairing insulin signaling and sensitizing the liver to inflammatory injury, induced by a variety of stimuli. Patients with NAFLD/NASH conditions typically present with chronic inflammation and other obesity-related risk factors, such as insulin resistance and hyperglycemia. Very interestingly, PPARα such as Pemafibrate, can not only increase the stability of atherosclerotic plaques, reduce the risk of atherothrombosis, slow the progression of intimal hyperplasia following surgery, but reduce hepatic fat accumulation and improved glucose metabolism significantly.¹¹ FGF21 can sensitize insulin and is produced mainly by the liver. In a phase-2 study, pemafibrate increased serum levels of FGF21 significantly. Pemafibrate tended to increase serum levels and mRNA expression of FGF21. An association of insulin resistance between muscle and adipose tissue cannot be excluded, but weight reduction, increase in energy expenditure, and alternation of FGF21 expression by pemafibrate may contribute to improve glucose metabolism. Note that NASH progresses with hyperinsulinemia and inhibition of the insulin receptor substrate (IRS).¹² A combination of hepatic insulin resistance, along with elevated serum glucagon that accompanies hyperinsulinemia due to pancreatic islet expansion, contributes to excessive production of glucose by the liver in diabetes and diabetic NASH. Thus, potent PPARα agonist appears to be a promising therapeutic agent for NAFLD/NASH, as well as dyslipidaemia and insulin resistance associated with NAFLD/NASH.

Inflammation represents a key component of the NAFLD/NASH. The pathological states of NASH increased inflammation and apoptosis relate factors in liver tissue. PPARα suppresses inflammation and the acute phase response and is discussed as a potential target for inflammatory disorders and to control obesity. PPARα negatively regulates pro-inflammatory and acute phase response (APR) signaling pathways, as seen in rodent models of systemic inflammation, atherosclerosis and NASH.¹³

PPARα ligand binds to the lipid binding domain enabling heterodimerisation with a ligand-activated retinoid X-receptor (RXR). The activation of PPARα causes induction or repression of the receptor's target genes, which leads to a slow but durable and extensive cellular response. During transrepression, the activated PPAR binds to cytokine activated transcription factors, such as nuclear factor kappa B or activator protein-1. Under normal conditions, these transcription factors induce the synthesis of proteins involved in the inflammatory response. PPARα can inhibit this process by blocking the interaction between activated transcription factors and the promoter region of the target gene, thereby preventing transcription and reducing inflammation. Very interestingly, another mechanism of PPARα-dependent transcriptional repression occurs in the control of ERR-driven mitochondrial respiration and cardiac contraction, where a PPARα-SIRT1 complex binds directly to a single hexameric ERRE motif, thus competitively downregulating ERR target genes.

However, the precise receptor-mediated response depends on the individual agonist and the tissue in which it is expressed. There is therefore an unmet medical need for a new generation of more potent and high affinity PPARα-agonists for limiting the progression of hepatic inflammatory in liver for NAFLD/NASH therapy.

Since activation of PPARα regulates expression of key genes involved in lipid homeostasis, targets for PPARα include key genes involved in lipid metabolism, such as apo A-I, A-II and A-V, LPL and SR-BI. Consequently, PPARα primarily regulates lipid homeostasis, whereas PPARγ largely regulates adipogenesis and glucose homeostasis.¹⁴ Since PPARα expression is enriched in hepatocytes, PPARα knockout mice increase the susceptibility to liver steatosis, inflammation, and hepatocellular carcinoma, suggested that PPARα has a protective role against NAFLD pathogenesis.¹⁵ Additionally, fat accumulation in the liver results from imbalanced metabolism of lipids. It was reported that expression of the PPARα gene in the human liver is correlated negatively with NASH severity and histologic improvement is associated with an increase in PPARα expression. Recent report indicated that the reduction in lipid outflow, fatty-acid oxidation and export of very-low-density lipoprotein are key factors in NASH pathogenesis.¹⁶ Recently, the effect of pemafibrate, a PPARα modulator, on rodent models of NASH was first investigated. Pemafibrate modulated lipid turnover and upregulated expression of UCP3 in the liver. In a phase-2 study, pemafibrate reduced plasma concentrations of liver enzymes (ALT and γ-glutamyl transferase) in patients with dyslipidaemia. This treatment was thought to increase ATP content in the liver by promoting fatty-acid oxidation, and to improve NASH pathogenesis by stimulating lipid turnover. Therefore, PPARα represents attractive drug targets for the management of NAFLD and NASH as well as related conditions such as type 2 diabetes and the broader metabolic syndrome. A potent PPARα modulator may have therapeutic efficacy against NAFLD/NASH.

It has recently been recognized that PPARα modulates inflammatory responses, regulates cell division and associates with liver fibrogenesis and tumorigenesis. Although the roles of PPARα for the pathogenesis of nonalcoholic steatohepatitis (NASH) remain unclear, several data suggest the relationship between PPARα and the occurrence or progression of NAFLD/NASH. For example, in mice lacking acyl-CoA oxidase gene, which developed NASH spontaneously, the absence of PPARα prevented the development of NASH.

The available data thus far indicate that PPARα can protect against NAFLD/NASH, the data also provides strong evidence for the intimate link between PPARα expression and NASH severity, note that whereas the expression of the other PPARs remain unaltered. PPARα protein appears to be more sensitive and is already reduced in steatotic livers, with a further reduction in NASH, suggested that PPARα expression in NASH livers may be caused by regulated genes involved in lipid and specific inflammatory mediators.⁵

We performed in vivo and cells experiments, liver gene expression of PPARα targets in mice treated at 3 mg/kg (except WY100=WY14643 at 100 mg/kg) with newly produced DY-PPARα agonists for 24 h. Data in same order observed as triglyceride suppression, apo CIII and LPL expression inversely correlate with triglyceride suppression for many compounds. ACOX1 and Cyp4A3 expression correlate with triglyceride suppression for many compounds. These data support the potential for selective modulation of PPARα in response to each PPARα modulator. Taken together, our preliminary data and recently published data provide strong support for the concept that PPARα protect from the development of NAFLD/NASH. PPARα activation by its agonists leads to upregulation of numerous genes involved in lipid metabolism, effectively reduces serum triglyceride levels in dyslipidemic subjects, significantly improves gluconeogenesis and hepatic insulin resistance in the liver. Thus, PPARα represents an exciting molecular target for the NAFLD/NASH therapy.

Interestingly, the potential for using synthetic small molecules to modulate its activity has been demonstrated. In the present study, a series of novel potent and selective PPARα agonists with an EC₅₀ of 0.85-12 nM for human PPARα has been identified. DY121 was identified as the most potent synthetic agonist for the PPARα with an EC₅₀ of 0.85 nM that exhibited excellent selectivity over PPARγ and PPARδ in PPAR-GAL4 transactivation assays. This potency and selectivity was also supported by computational docking models that suggest DY121 forms more extensive pi-stacking and pi-anion interactions with PPARα in ligand binding domain which should result in higher binding affinity on PPARα over PPARγ and PPARδ. Compound DY121 demonstrated excellent in vivo efficacy and safety profiles in preclinical studies and thus was chosen for further preclinical evaluation.

Since PPARα appears to be protective against NAFLD/NASH, it follows that ligands that bind to and activate PPARα may be potential therapeutic agents. In the present study, a series of novel potent and selective PPARα agonists with an EC₅₀ of 0.85-12 nM for human PPARα assays has been identified. Among them, DY121 was identified with an EC₅₀ of 1 nM for human PPARα in PPAR-GAL4 transactivation assays. DY121 demonstrated excellent in vivo efficacy and safety profiles in preclinical studies and thus was chosen for further preclinical animal toxicity evaluation. The synthesis, structure-activity relationship (SAR) studies, and in vivo pharmacology of DY121 and its analogs in preclinical animal models as well as its molecular modeling profile are described.

During the investigation of ligands as potential therapeutic agents in our laboratory, we have developed a series of novel highly potent human PPARα activators with a high selectivity for PPARα over PPARγ and PPARδ. This work led to the discovery of compound DY121 (compound 10a) as the most potent agonist among those compounds studied. Our SAR studies demonstrate that para-substituted methoxy and methyl groups, or meta-substituted chlorine group of the benzyl ring at the N⁴ position in the 2,4-dihydro-3H-1,2,4-triazol-3-one system provide more flexibility and lead to more potent PPARα activators. Animal studies indicated that DY121 may be a selective modulator of PPARα that lowers triglycerides without raising glucose. Studies are continuing in PPARα^{-/-} α. Since DY121 shows promise in treating the metabolic syndrome such as NAFLD. To develop DY121 into a drug for clinical use, further investigation of its potential off-target effects, toxicity, pharmacokinetics, bioavailability, and ADME (absorption, distribution, metabolism, excretion) is needed.

### Example 1. Design & Synthesis of Novel PPARα-Selective Modulators

A range of synthetic PPARα agonists, differing in species specific potencies and efficacies, have been identified recently. There are currently several synthetic PPARα ligands from the fibrate class of hypolipidemic drugs in clinical use. In humans, the triglyceride-lowering effect of fibrates is attributed to the activation of PPARα which in turn effects an increase in lipoprotein lipase gene expression and transrepression of apoC-III. Also, ureidothioisobutyric acid, GW 9578 and GW7647 (FIG. 1) were reported as potent and subtype-selective PPARα agonists that have an improved lipid-lowering activity over that of fenofibrate.¹⁷ The identification of human PPARα selective a alkylphenylpropanoic acid derivatives has been reported by Miyachi et al.¹⁸ Interestingly, a new series of hPPARα agonists containing a triazolone core was described that led to the discovery of LY518674, a highly potent and selective agonist with an EC₅₀ of 42 nM against PPARα (FIG. 1).¹⁹ Despite these advances, the development of PPARα agonists with better potency and selectivity remains a formidable challenge.

As a part of our ongoing program to explore novel classes of PPARα modulators with the goal of increasing the potency and selectivity for PPARα subtype which might prove to be of therapeutic value in treating NASH/NAFLD, we applied a strategy to take advantage of altering the structure of LY518674, since it displayed potent and selective binding affinity and functional activity on the hPPARR receptor subtype and exhibited highly potent in vivo efficacy in a human apoA-I transgenic mouse model. Note that in the structure-activity relationship (SAR) of LY518674 of this series of hPPARα agonists, the authors provided evidence that the linker length of three carbons in the triazolone core were consistent with activity. Also, an N²-benzyl substituent of the triazolone core is attributed to the activation of PPARα. However, the authors indicated that the substituent effects of the N⁴ position of LY seems to be less pronounced. For example, they reported that in hPPARα functional potency, incorporation of an acetic acid moiety at N⁴ position resulted in a sharp loss in hPPARα affinity and potency. It has been reported that the unsubstituted analogue at N⁴ position of LY518674 is a potent and selective on hPPARα.

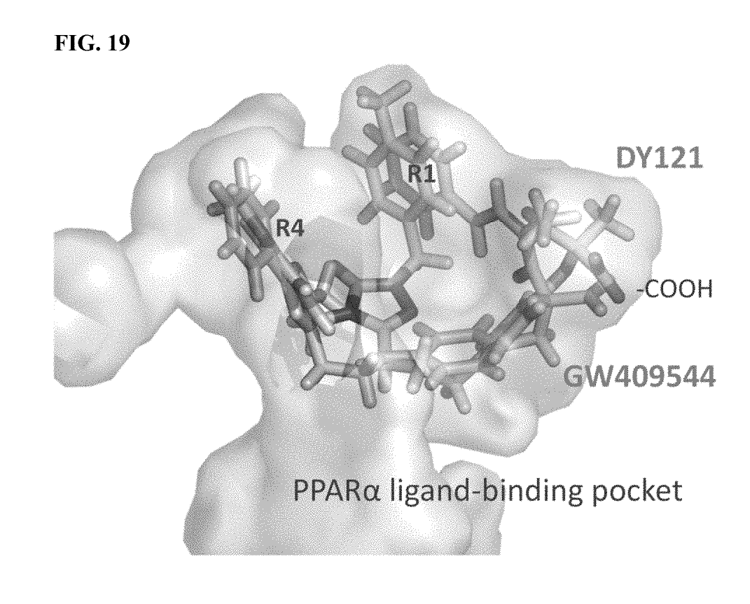

However, we noticed that our SAR studies are somewhat different from LY518674, that is, the hydrophobic pocket of hPPARα has 3 major interaction regions R1 region/R4 region/—COOH overlaps with X-ray ligand with the ligand GW409544 (dark grey-colored) from X-ray structure in PDB 1k71 (FIG. 19), clearly indicated that the steric bulkiness and position of the substituent of the N⁴ position (R4 region) are critical for PPARα agonistic activity and selectivity. Since the analogs bearing extension or branched benzyl groups at the N⁴ position of the triazolone core could exhibit improved binding affinity and selectivity profiles for PPARα and produce even more potent compounds as compared to LY518674, we anticipated that if we could introduce a sufficiently bulky substituent instead of the H group at the N⁴ of LY. By this way, we would be able to create a hPPARα potent and selective modulator, because the introduced benzyl substituent at the N⁴ would enhance the interaction with the hydrophobic pocket of the hPPARα LBD with more pi-stacking and pi-anion interactions. Based on this hypothesis, we decided to investigate the template of the LY, especially to alter the branched benzyl moiety of N⁴ position to generate higher potency and affinity PPARα modulators since it is into its appropriate binding pocket at the hydrophobic R4 region part.

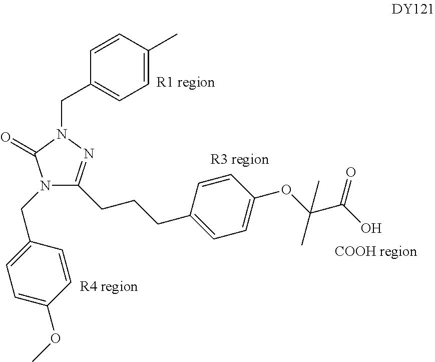 [SMILES: COc1ccc(Cn2c(CCCc3ccc(OC(C)(C)C(=O)O)cc3)nn(Cc3ccc(C)cc3)c2=O)cc1]

The synthetic routes to a series of compounds are outlined in Scheme 1, 2 and 3. A series of novel compounds containing branched benzyl moiety at the N⁴ position of a triazolone core was synthesized and evaluated for PPARα activity. The newly produced derivatives, exemplified by 2-(4-{3-(4-methoxy-benzyl)-1-(4-methyl-benzyl-5-oxo-4,5-dihydro-1H{1,2,4}triazol-3-yl}-propyl)-phenoxy)-2-methyl-propionic acid (10a, DY121), are highly potent agonists that modulate the activity PPARα. Its analogs 2-(4-{3-(4-methoxy-benzyl)-1-(4-methyl-benzyl-5-oxo-4,5-dihydro-1H{1,2,4}triazol-3-yl}-propyl)-phenoxy)-2-methyl-propionic acid derivatives (10a-g) were synthesized used the same method. Commercially available 4-(4-methoxyphenyl)-butyric acid (1) was esterified to obtain methyl-4-(4-methoxyphenyl)-butyrate (2). Demethylation of (2) by ether cleavage with boron tribromide gave the hydroxyl derivative 4-(4-hydroxyphenyl)butyric acid methyl ester (3). Compound 3 was alkylated with tert-butylbromoisobutyrate to obtain the diester (4), which was converted to the hydrazide (5) with hydrazine hydrate. The hydrazide (5) was then reacted with a variety of substituted benzylisocyanates to give the series of acylsemicarbazide derivatives (6a-g) for different benzyl substituents introduced at the N⁴ position. Treatment with KOH/MeOH led to ring closure to form the trizolone core with the simultaneous hydrolysis of the tert-butyl ester (7a-g). The acids (7a-g) were re-esterified with methanol and concentrated sulfuric acid to produce the methyl esters (8a-g). The N² of the 8a-g series was then alkylated by treating with 4-methylbenzyl bromide in the presence of K₂CO₃ in DMF to give the series (9a-g). The final products (10a-g) were prepared by ester hydrolysis using sodium hydroxide in methanol. In Scheme 2, a variation of the substituted benzyl derivative was synthesized by alkylating 8e with 4-vinylbenzyl chloride to give 9 h. Subsequent hydrolysis of the ester group provided 10 h.

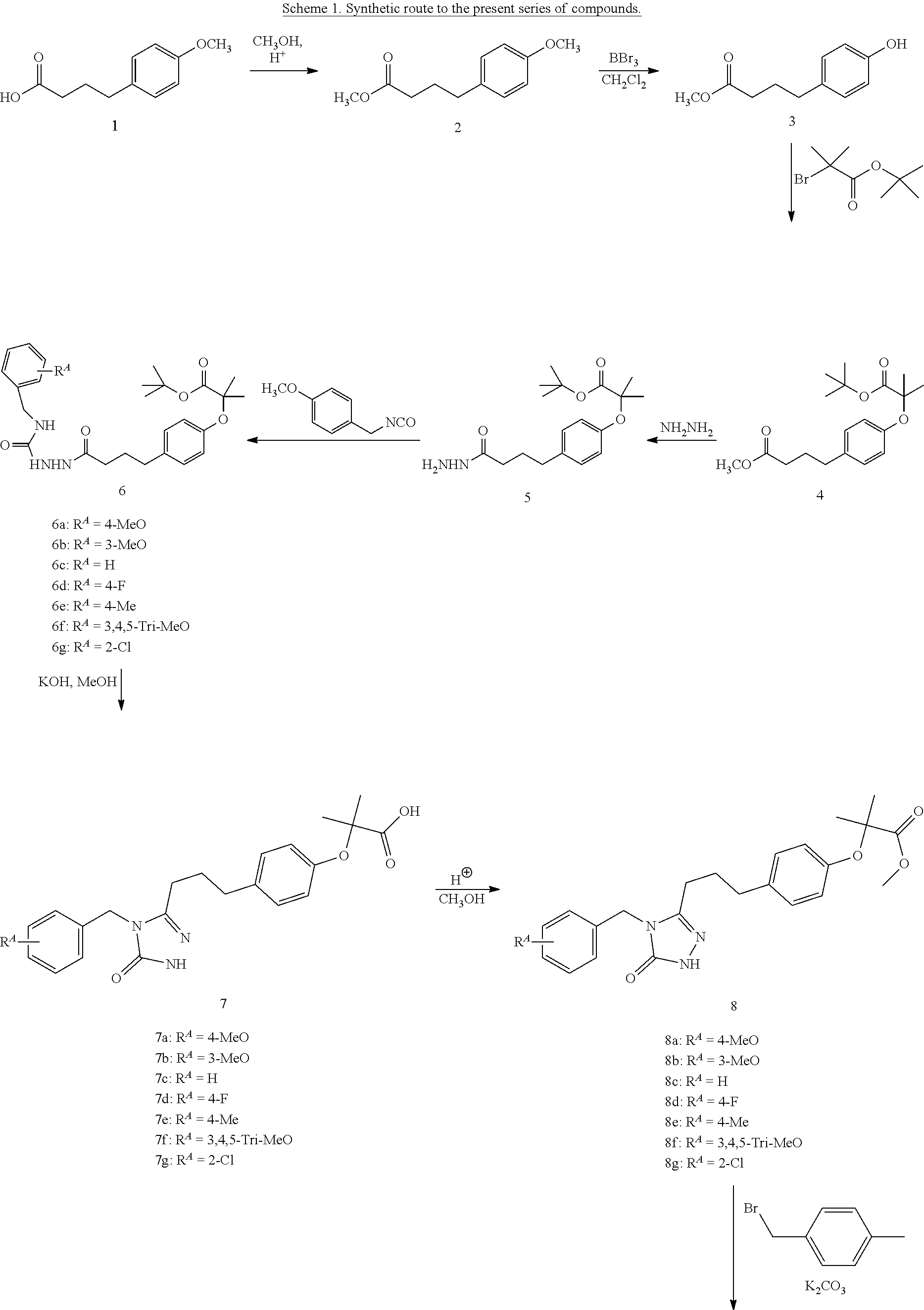

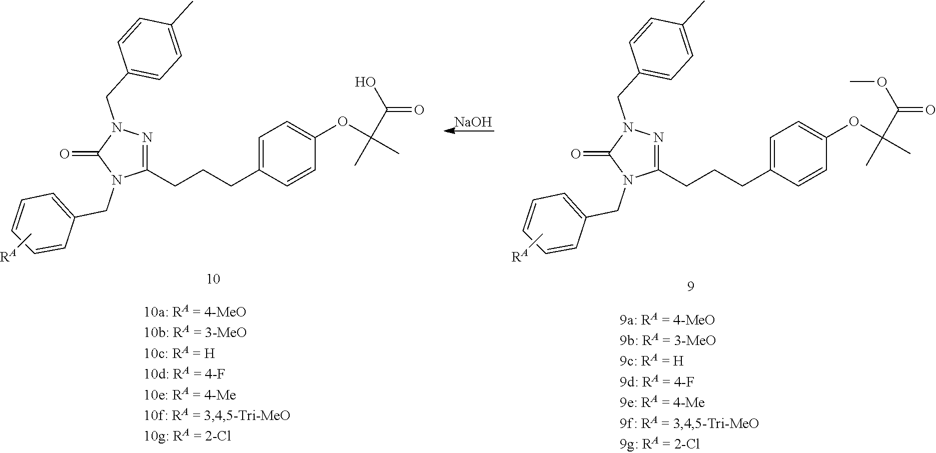

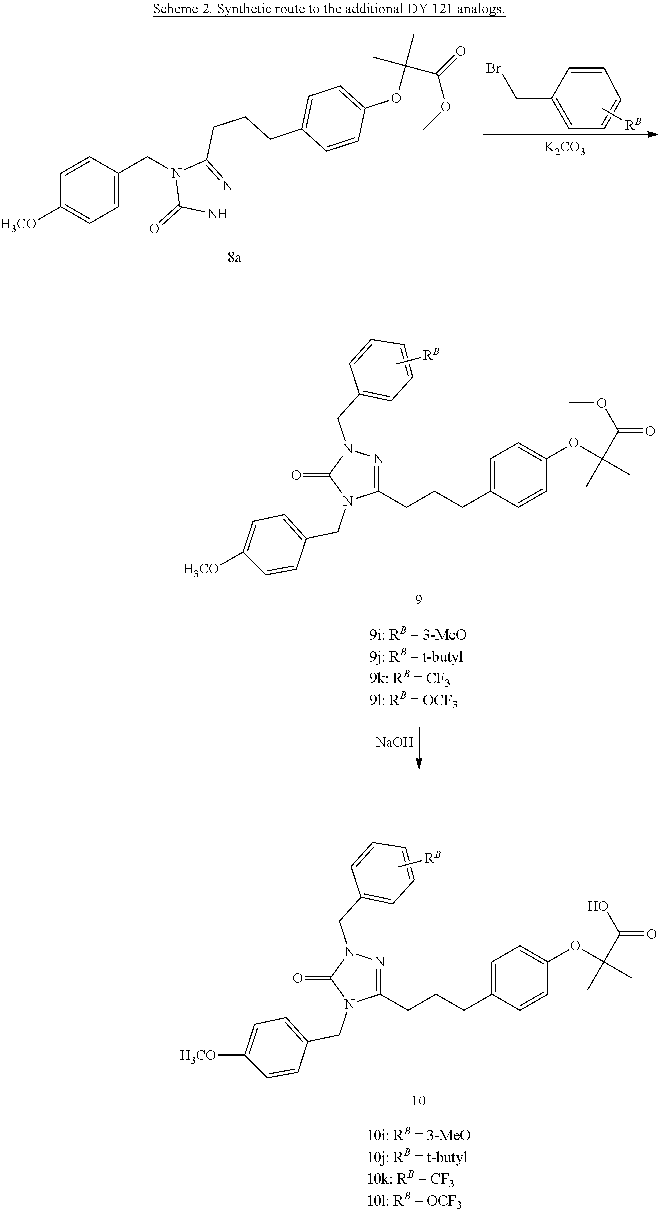

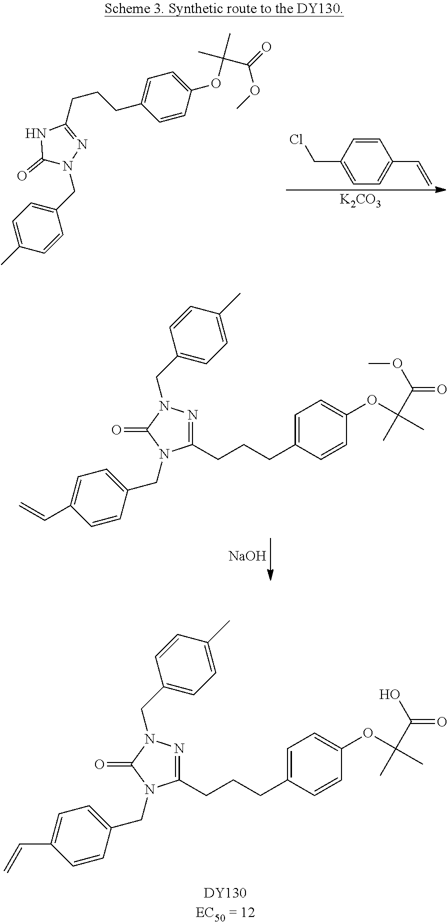

General procedures: Organic reagents were purchased from commercial suppliers unless otherwise noted and were used without further purification. All solvents were analytical or reagent grade. All reactions were carried out in flame-dried glassware under argon or nitrogen. Melting points were determined and reported automatically by an optoelectronic sensor in open capillary tubes and were uncorrected. ¹H NMR and ¹³C NMR spectra were measured at 500 MHz and 125 MHz respectively, and using CDCl₃ or CD₃OD as the solvents and tetramethylsilane (Me₄Si) as the internal standard. Liquid column chromatography was carried out under moderate pressure by using columns of an appropriate size packed and eluted with appropriate eluents. All reactions were monitored by TLC on precoated plates (silica gel HLF). TLC spots were visualized either by exposure to iodine vapors or by irradiation with UV light. Organic solvents were removed in vacuum by rotary evaporator. Elemental analyses were performed by Desert Analytics, Tucson, Ariz.

### Methyl 4-(4-methoxyphenyl)butyrate (2)

To a solution of 4(4-Methoxyphenyl)-butyric acid (1) (10 g, 0.051 mol) in methanol (200 mL) was added concentrated sulfuric acid (1 mL) and the reaction mixture stirred at room temperature for 16 h. After concentration to remove methanol, the oil was dissolved in ethyl acetate (120 mL), washed with Na₂CO₃ (3×70 mL), water (100 mL), and brine (100 mL). The organic layer was dried (Na₂SO₄) and concentrated to give the methyl ester as an oil (10.34 g) in 97% yield. ¹H NMR (CDCl₃) δ 7.12 (d, 2H), 6.85 (d, 2H), 3.77 (s, 3H), 3.69 (s, 3H), 2.62 (t, 2H), 2.59 (t, 2H), 1.96 (t, 2H); ¹³C NMR (CDCl₃) δ 173.9, 157.2, 133.3, 129.8, 128.7, 114.1, 113.1, 56.5, 53.6, 34.1, 33.2, 26.6.

### 4-(4-Hydroxy-phenyl)-butyric acid methyl ester (3)

BBr₃ (80 mL, 1.0 M solution in dichloromethane) was added dropwise to a cooled (0° C.) solution of methyl 4-(4-methoxyphenyl)butyrate (2) (10.34 g, 0.05 mol) in dried CH₂Cl₂ (60 mL) under Ar. After stirring for an additional hour at 0° C., the reaction mixture was treated with 1:1 CH₃OH: CH2Cl2 (80 mL) with cooling and stirred overnight at ambient temperature. Concentration of the mixture gave oil, which portioned between ethyl acetate (100 ml) and water (100 ml). The aqueous layer was extracted with EtOAc (2×50 mL), and the combined organic extracts washed with water (50 mL), brine (50 mL), dried over Na₂SO₄, then concentrated to give the desired phenol as an oil 9.88 g which was purified by flash chromatography (8:2 hexanes/EtOAc) afforded 3 (9.19 g, 98%) as pale yellow oil. ¹H NMR (CDCl₃) δ 7.01 (d, 2H), 6.80 (d, 2H), 3.68 (s, 3H), 2.55 (t, 2H), 2.34 (t, 2H), 1.93 (t, 2H); ¹³C NMR (CDCl₃) δ 174.8, 154.0, 132.8, 129.3, 115.1, 51.6, 34.0, 33.5, 26.5.

### 4-[4-(1-tert-Butoxycarbonyl-1-methyl-ethoxy)-phenyl]-butyric acid methyl ester (4)

4-(4-Hydroxy-phenyl)-butyric acid methyl ester (3) (4.94.19 g, 0.025 mol) was dissolved in DMF (80 mL) and treated with t-butyl 2-bromoisobutyrate (14 mL, 0.081 mol), powder K₂CO₃ (14 g, 0.10 mol) and MgSO₄ (0.5 g, 0.0042 mol), and the resulting mixture heated at 75° C. overnight. After cooling to ambient temperature, the reaction mixture was filtered into 1N aqueous HCl (80 mL) and extracted with EtOAc (3×50 mL). The remaining solids from the filtration were washed several times with EtOAc. The EtOAc extracts and washers were combined and washed with 1N aqueous HCl (40 mL), dried over Na₂SO₄, and concentrated to dark oil. Purification by flash chromatography (gradient elution, hexanes to 95:5 H/EtOAc) gave the desired ether (4) (6.03 g) in 82% as oil. ¹H NMR (CDCl₃) δ 7.02 (d, 2H), 6.77 (d, 2H), 3.64 (s, 3H), 2.55 (t, 2H), 2.29 (t, 2H), 1.89 (t, 2H), 1.52 (s, 6H), 1.43 (s, 9H); ¹³C NMR (CDCl₃) δ 173.9, 173.3, 153.8, 134.5, 128.8, 118.9, 81.4, 79.3, 51.4, 34.2, 33.3, 28.0, 26.6, 25.3.

### 2-[4-(3-Hydrazinocarbonyl-propyl)-phenoxy]-2-methyl-propionic acid tert-butyl ester (5)

A solution of 4-[4-(1-tert-Butoxycarbonyl-1-methyl-ethoxy)-phenyl]-butyric acid methyl ester (4). (6.03 g, 0.018 mol) in methanol (30 mL) was treated with hydrazine hydrate (5.99 g, 0.12 mol) and the mixture stirred overnight at ambient temperature. The reaction mixture was concentrated and the residue partitioned between EtOAc (50 mL) and water (20 mL). The aqueous layer was extracted with EtOAc (2×20 mL), and the combined organic extracts washed with brine (20 mL), dried over Na₂SO₄, and concentrated to give the desired hydrazide as an oil which was solidified by pet ether gave white solid 5.15 g in 85% yield, mp: 74.5° C. ¹H NMR (CDCl₃) δ 7.37 (s, 1H), 6.97 (d, 2H), 6.72 (d, 2H), 3.85 (s, 2H), 2.52 (t, 2H), 2.09 (t, 2H), 1.89 (t, 2H), 1.49 (s, 6H), 1.40 (s, 9H); ¹³C NMR (CDCl₃) δ 173.6, 173.3, 153.8, 134.5, 128.8, 119.0, 81.5, 79.3, 34.2, 34.5, 28.0, 26.9, 25.3.

### 2-(4-{N′-4-methoxy-benzylaminocarbonyl)-hydrazino]-4-oxo-butyl}-phenoxy)-2-methyl-propionic acid tert-butyl ester (6a)

To a solution of crude 2-[4-(3-Hydrazinocarbonyl-propyl)-phenoxy]-2-methyl-propionic acid tert-butyl ester (5) (5.15 g, 0.015 mol) in anhydrous dichloromethane (100 mL) was added 4-methoxybenzyl isocyanate (3.35 g, 0.02 mol) dropwise. The reaction mixture was stirred for two hours at ambient temperature, and then concentrated to give the desired acylsemicarbazide as solid. Purification by pet ether gave white solid 6.37 g in 85% yield, mp: 131.2° C. ¹H NMR (CDCl₃) δ 7.71 (s, 1H), 7.17 (d, 2H), 7.07 (d, 1H), 6.98 (d, 2H), 6.81 (d, 2H), 6.74 (d, 2H), 5.66 (d, 1H), 4.27 (d, 2H), 3.78 (s, 3H), 2.53 (t, 2H), 2.16 (t, 2H), 1.89 (t, 2H), 1.53 (s, 6H), 1.43 (s, 9H); ¹³C NMR (CDCl₃) δ 174.9, 174.0, 160.2, 159.4, 155.2, 135.8, 132.1, 130.2, 129.8, 120.5, 115.9, 83.1, 80.7, 56.6, 45.5, 35.6, 34.6, 29.3, 28.0, 26.7. Anal. Calcd for C₂₇H₃₇N₃O₆: C, 64.91; H, 7.46; N, 8.41. Found: C, 64.88; H, 7.29; N, 8.53.

### 2-(4-{N′-3-methoxy-benzylaminocarbonyl)-hydrazino]-4-oxo-butyl}-phenoxy)-2-methyl-propionic acid tert-butyl ester (6b)

To a solution of crude 2-[4-(3-Hydrazinocarbonyl-propyl)-phenoxy]-2-methyl-propionic acid tert-butyl ester (5) (0.86 g, 0.026 mol) in anhydrous dichloromethane (20 mL) was added 4-methoxybenzyl isocyanate (0.55 g, 0.0034 mol) dropwise. The reaction mixture was stirred for two hours at ambient temperature, and then concentrated to give the desired acylsemicarbazide as solid. Purification by pet ether gave white solid 1.12 g in 86% yield, mp: 87.9° C. ¹H NMR (CDCl₃) δ 7.94 (s, 1H), 7.34 (s, 1H), δ 7.18 (s, 1H), 6.98 (d, 2H), 6.80 (m, 5H), 5.82 (t, 1H), 4.30 (d, 2H), 3.76 (s, 3H), 2.53 (t, 2H), 2.16 (t, 2H), 1.90 (t, 2H), 1.56 (s, 6H), 1.45 (s, 9H); ¹³C NMR (CDCl₃) δ 179.2, 177.0, 171.5, 159.8, 156.3, 148.2, 136.7, 134.7, 129.2, 120.0, 119.2, 113.8, 78.8, 61.7, 56.4, 45.2, 34.6, 33.8, 29.0, 28.0, 26.3. Anal. Calcd for C₂₇H₃₇N₃O₆: C, 64.91; H, 7.46; N, 8.41. Found: C, 65.00; H, 7.34; N, 8.45.

### 2-(4-{N′-benzylaminocarbonyl)-hydrazino]-4-oxo-butyl}-phenoxy)-2-methyl-propionic acid tert-butyl ester (6c)

To a solution of crude 2-[4-(3-Hydrazinocarbonyl-propyl)-phenoxy]-2-methyl-propionic acid tert-butyl ester (5) (1.07 g, 0.0032 mol) in anhydrous dichloromethane (30 mL) was added benzyl isocyanate (0.56 g, 0.0042 mol) dropwise. The reaction mixture was stirred for five hours at ambient temperature, and then concentrated to give the desired acylsemicarbazide as a solid. Crystallization by pet ether gave a white solid (1.07 g) in 73% yield, mp: 124.9° C. ¹H NMR (CDCl₃) δ 7.99 (s, 1H), 7.39 (s, 1H), 7.23 (m, 5H), 6.98 (d, 2H), 6.76 (d, 1H), 5.86 (s, 1H), 4.33 (d, 2H), 2.54 (t, 2H), 2.16 (t, 2H), 1.88 (t, 2H), 1.53 (s, 6H), 1.45 (s, 9H); ¹³C NMR (CDCl₃) δ 170.5, 169.7, 155.1, 151.0, 135.7, 131.5, 126.0, 125.7, 125.4, 124.5, 124.4, 116.2, 78.8, 76.5, 41.7, 41.2, 31.3, 30.3, 24.9, 23.7, 22.5. Anal. Calcd for C₂₆H₃₅N₃O₅: C, 66.50; H, 7.51; N, 8.95. Found: C, 66.46; H, 7.33; N, 9.20.

### 2-(4-{N′-4-fluoro-benzylaminocarbonyl)-hydrazino]-4-oxo-butyl}-phenoxy)-2-methyl-propionic acid tert-butyl ester (6d)

To a solution of crude 2-[4-(3-Hydrazinocarbonyl-propyl)-phenoxy]-2-methyl-propionic acid tert-butyl ester (5) (0.5 g, 0.0015 mol) in anhydrous dichloromethane (10 mL) was added 4-fluorobenzyl isocyanate (0.3 g, 0.002 mol) dropwise. The reaction mixture was stirred for twelve hours at ambient temperature, and then concentrated to give the desired acylsemicarbazide as a solid. Solidification by pet ether gave a white solid (0.52 g) in 71% yield, mp: 99.9° C. ¹H NMR (CDCl₃) δ 7.83 (s, 1H), 7.23 (m, 3H), 6.98 (m, 4H), 6.75 (d, 2H), 5.82 (t, 1H), 4.32 (t, 2H), 2.55 (t, 2H), 2.17 (t, 2H), 1.90 (t, 2H), 1.56 (s, 6H), 1.44 (s, 9H); ¹³C NMR (CDCl₃) δ 170.6, 169.8, 155.0, 150.9, 135.6, 131.4, 126.1, 126.0, 125.9, 116.1, 115.9, 112.5, 112.4, 112.3, 78.8, 76.4, 40.3, 31.2, 30.2, 24.9, 24.7, 23.6, 22.4, 22.1. Anal. Calcd for C₂₆H₃₄FN₃O₅: C, 64.05; H, 7.03; N, 8.62; F, 3.90. Found: C, 63.98; H, 7.17; N, 8.59; F, 3.57.

### 2-(4-{N′-4-methyl-benzylaminocarbonyl)-hydrazino]-4-oxo-butyl}-phenoxy)-2-methyl-propionic acid tert-butyl ester (6e)

To a solution of crude 2-[4-(3-Hydrazinocarbonyl-propyl)-phenoxy]-2-methyl-propionic acid tert-butyl ester (5) (1.0 g, 0.003 mol) in anhydrous dichloromethane (20 mL) was added 4-methylbenzyl isocyanate (0.60 g, 0.0041 mol) dropwise. The reaction mixture was stirred for twelve hours at ambient temperature, and then concentrated to give the desired acylsemicarbazide as solid. Purification by pet ether gave white solid 1.17 g in 81% yield, mp: 139.9° C. ¹H NMR (CDCl₃) δ 8.27 (s, 1H), 7.60 (s, 1H), δ 7.10 (dd, 4H), 6.97 (d, 2H), 6.75 (d, 2H), 5.92 (t, 1H), 4.25 (d, 2H), 2.51 (t, 2H), 2.31 (s, 3H), 2.15 (t, 2H), 1.85 (t, 2H), 1.52 (s, 6H), 1.43 (s, 9H); ¹³C NMR (CDCl₃) δ 179.2, 170.6, 169.6, 155.1, 150.8, 133.9, 132.6, 131.6, 126.3, 126.0, 124.5, 116.1, 78.7, 76.4, 40.8, 31.3, 30.2, 24.9, 23.7, 22.4, 18.1. Anal. Calcd for C₂₇H₃₇N₃O₅: C, 67.06; H, 7.71; N, 8.69. Found: C, 67.16; H, 7.77; N, 8.59.

### 2-(4-{N′-3,4,5-trimethoxy-benzylaminocarbonyl)-hydrazino]-4-oxo-butyl}-phenoxy)-2-methyl-propionic acid tert-butyl ester (6f)

To a solution of crude 2-[4-(3-Hydrazinocarbonyl-propyl)-phenoxy]-2-methyl-propionic acid tert-butyl ester (5) (1.09 g, 0.0032 mol) in anhydrous dichloromethane (20 mL) was added 3,4,5-trimethoxybenzyl isocyanate (0.94 g, 0.0042 mol). The reaction mixture was stirred for twelve hours at ambient temperature, and then concentrated to give the desired acylsemicarbazide as an oil. Solidification by ether gave white solid 1.43 g in 80% yield, mp: 92.0° C. ¹H NMR (CDCl₃) δ 7.75 (s, 1H), 7.06 (s, 1H), 6.70 (d, 2H), 6.76 (d, 2H), 6.47 (s, 2H), 5.75 (t, 1H), 4.28 (d, 2H), 3.81 (s, 9H), 2.56 (t, 2H), 2.18 (s, 3H), 1.92 (t, 2H), 1.53 (s, 6H), 1.44 (s, 9H); ¹³C NMR (CDCl₃) δ 170.6, 170.1, 155.2, 151.0, 150.4, 131.5, 131.4, 126.1, 116.1, 101.4, 78.9, 76.4, 57.9, 53.2, 41.3, 31.3, 30.3, 24.9, 23.6, 22.5. Anal. Calcd for C₂₉H₄₁N₃O₈: C, 62.24; H, 7.38; N, 7.51. Found: C, 62.23; H, 7.17; N, 7.25.

### 2-(4-{N′-2-Chloro-benzylaminocarbonyl)-hydrazino]-4-oxo-butyl}-phenoxy)-2-methyl-propionic acid tert-butyl ester (6 g)

To a solution of crude 2-[4-(3-Hydrazinocarbonyl-propyl)-phenoxy]-2-methyl-propionic acid tert-butyl ester (5) (1.59 g, 0.0047 mol) in anhydrous dichloromethane (40 mL) was added 2-chlorobenzyl isocyanate (1.06 g, 0.0063 mol). The reaction mixture was stirred for twelve hours at ambient temperature, and then concentrated to give the desired acylsemicarbazide as oil. Solidification by pet ether gave white solid 1.85 g in 78% yield, mp: 141.3° C. ¹H NMR (CDCl₃) δ 7.80 (s, 1H), 7.35 (m, 4H), 7.18 (s, 1H), 6.70 (d, 2H), 6.76 (d, 2H), 5.89 (t, 1H), 4.44 (d, 2H), 2.56 (t, 2H), 2.19 (s, 3H), 1.92 (t, 2H), 1.52 (s, 6H), 1.45 (s, 9H); ¹³C NMR (CDCl₃) δ 170.5, 169.6, 151.0, 131.4, 126.6, 126.5, 126.0, 125.8, 124.1, 116.2, 78.8, 76.5, 39.0, 31.3, 30.4, 24.9, 23.7, 22.5. Anal. Calcd for C₂₆H₃₄ClN₃O₅: C, 61.96; H, 6.80; N, 8.34; Cl, 7.03. Found: C, 62.30; H, 6.71; N, 8.33; Cl, 6.89.

### 2-(4-{3-[4-(4-Methoxy-benzyl)-5-oxo-4, 5-dihydro-1H-[1,2,4]triazol-3-yl]-propyl}-phenoxy)-2-methyl-propionic acid (7a)

To a solution of 2-(4-{N′-4-methoxy-benzylaminocarbonyl)-hydrazino]-4-oxo-butyl}-phenoxy)-2-methyl-propionic acid tert-butyl ester (6a) (5.0 g, 0.01 mol) in methanol (50 mL) was added solid potassium hydroxide (8.0 g, 0.14 mol) and the reaction mixture heated at reflux for 48 hours. After cooling to ambient temperature, the reaction mixture was diluted with water (50 mL) and EtOAc (150 mL), and then acidified to pH 2 with 5N HCl. The aqueous layer was extracted with EtOAc (2×25 mL). The combined organic extracts were washed with water (70 mL), brine (70 mL), dried over Na₂SO₄, and concentrated to give the desired triazolinone as an oil, which was solidified by ether to afford white solid 4.15 g in 98% yield, mp: 143.3° C. ¹H NMR (CDCl₃) δ 10.06 (brs, 1H), 7.11 (d, 2H), 6.95 (d, 2H), 6.85 (m, 4H), 4.67 (s, 2H), 3.79 (s, 3H), 2.57 (t, 2H), 2.38 (t, 2H), 1.89 (t, 2H), 1.61 (s, 6H); ¹³C NMR (CDCl₃) δ 174.3, 156.5, 153.4, 150.4, 145.3, 131.9, 126.2, 125.8, 124.6, 116.6, 111.5, 76.4, 52.4, 41.0, 31.0, 24.1, 22.4, 22.2. Anal. Calcd for C₂₃H₂₇N₃O₅: C, 64.93; H, 6.40; N, 9.88. Found: C, 64.72; H, 6.31; N, 9.76.

### 2-(4-{3-[4-(3-Methoxy-benzyl)-5-oxo-4, 5-dihydro-1H-[1,2,4]triazol-3-yl]-propyl})-phenoxy)-2-methyl-propionic acid (7b)

To a solution of 2-(4-{N′-3-methoxy-benzylaminocarbonyl)-hydrazino]-4-oxo-butyl}-phenoxy)-2-methyl-propionic acid tert-butyl ester (6b) (1.11 g, 0.0022 mol) in methanol (15 mL) was added solid potassium hydroxide (1.85 g, 0.033 mol) and the reaction mixture heated at reflux for 36 hours. After cooling to ambient temperature, the reaction mixture was diluted with water (10 mL) and CH₂Cl₂ (30 mL), and then acidified to pH 2 with 5N HCl. The aqueous layer was extracted with CH₂Cl₂ (3×20 mL). The combined organic extracts were washed with water (30 mL), brine (30 mL), dried over Na₂SO₄, and concentrated to give the desired triazolinone as a white solid which was crystallized by MeOH to afford a white crystals 0.67 g in 71% yield, mp: 154.9° C. ¹H NMR (CDCl₃) δ 10.76 (s, 1H), 7.21 (t, 1H), 6.92 (d, 2H), 6.79 (t, 3H), 6.67 (d, 2H), 4.69 (s, 2H), 3.73 (s, 3H), 2.52 (t, 2H), 2.35 (t, 2H), 2.07 (s, 3H), 2.02 (s, 3H), 1.83 (t, 2H); ¹³C NMR (CDCl₃) δ 178.8, 159.8, 156.3, 153.2, 148.2, 136.6, 134.7, 130.4, 128.8, 120.0, 119.0, 113.8, 78.8, 55.2, 44.7, 33.8, 27.0, 25.2, 21.3. Anal. Calcd for C₂₃H₂₇N₃O₅: C, 64.93; H, 6.40; N, 9.88. Found: C, 64.70; H, 6.25; N, 9.89.

### 2-(4-{3-[4-Benzyl-5-oxo-4,5-dihydro-1H-[1,2,4]triazol-3-yl]-propyl)-phenoxy)-2-methyl-propionic acid (7c)

To a solution of 2-(4-{N′-benzylaminocarbonyl)-hydrazino]-4-oxo-butyl}-phenoxy)-2-methyl-propionic acid tert-butyl ester (6c) (1.07 g, 0.0023 mol) in methanol (30 mL) was added solid potassium hydroxide (2.00 g, 0.036 mol) and the reaction mixture heated at reflux for 36 hours. After cooling to ambient temperature, the reaction mixture was diluted with water (30 mL) and CH₂Cl₂ (100 mL), and then acidified to pH 2 with 5N HCl. The aqueous layer was extracted with CH₂Cl₂ (3×30 mL). The combined organic extracts were washed with water (30 mL), brine (30 mL), dried over Na₂SO₄, and concentrated to give the desired triazolinone as semi solid which was crystallization by EtOAc to afford white crystals 0.81 g in 89% yield, mp: 110.4° C. ¹H NMR (CDCl₃) δ 10.39 (s, 1H), 7.36 (m, 3H), 7.19 (t, 2H), 6.96 (d, 2H), 6.84 (t, 2H), 4.77 (s, 2H), 2.57 (t, 2H), 2.39 (t, 2H), 2.07 (t, 2H), 1.62 (s, 6H); ¹³C NMR (CDCl₃) δ 174.6, 153.5, 150.4, 145.4, 132.4, 131.9, 126.2, 126.1, 125.3, 124.3, 116.7, 76.3, 41.5, 30.9, 24.1, 22.4, 22.1. Anal. Calcd for C₂₂H₂₅N₃O₅: C, 66.82; H, 6.37; N, 10.63. Found: C, 66.66; H, 6.40; N, 10.39.

### 2-(4-{3-[4-(4-Fluoro-benzyl)-5-oxo-4, 5-dihydro-1H-[1,2,4]triazol-3-yl]-propyl})-phenoxy)-2-methyl-propionic acid (7d)

To a solution of 2-(4-{N′-4-fluoro-benzylaminocarbonyl)-hydrazino]-4-oxo-butyl}-phenoxy)-2-methyl-propionic acid tert-butyl ester (6d) (0.52 g, 0.001 mol) in methanol (15 mL) was added solid potassium hydroxide (0.84 g, 0.015 mol) and the reaction mixture heated at reflux for 32 hours. After cooling to ambient temperature, the reaction mixture was diluted with water (10 mL) and EtOAc (30 mL), and then acidified to pH 2 with 5N HCl. The aqueous layer was extracted with ETOAc (2×15 mL). The combined organic extracts were washed with water (30 mL), brine (30 mL), dried over Na₂SO₄, and concentrated to give the desired triazolinone as an oil which was solidified by chloroform to afford white solid 0.43 g in 98% yield, mp: 159.7° C. ¹H NMR (CD₃OD) δ 7.19 (m, 2H), 7.08 (m, 2H), 6.99 (d, 3H), 6.96 (d, 2H), 4.76 (s, 2H), 2.56 (t, 2H), 2.42 (t, 2H), 1.82 (t, 2H), 1.52 (s, 6H); ¹³C NMR (CDCl₃) δ 173.5, 160.4, 158.5, 153.1, 150.7, 154.5, 132.0, 129.3, 129.2, 126.0, 125.9, 125.8, 116.4, 112.5, 112.3, 75.8, 40.5, 32.7, 23.7, 21.2, 21.0. Anal. Calcd for C₂₂H₂₄FN₃O₄.¼H₂O: C, 63.22; H, 5.91; F, 4.55; N, 10.05. Found: C, 63.30; H, 5.68; N, 9.88; F, 4.29.

### 2-(4-{3-[4-(4-Methyl-benzyl)-5-oxo-4, 5-dihydro-1H-[1,2,4]triazol-3-yl]-propyl})-phenoxy)-2-methyl-propionic acid (7e)

To a solution of 2-(4-{N′-4-methyl-benzylaminocarbonyl)-hydrazino]-4-oxo-butyl}-phenoxy)-2-methyl-propionic acid tert-butyl ester (6e) (1.07 g, 0.022 mol) in methanol (20 mL) was added solid potassium hydroxide (1.85 g, 0.033 mol) and the reaction mixture heated at reflux for 32 hours. After cooling to ambient temperature, the reaction mixture was diluted with water (15 mL) and EtOAc (30 mL), and then acidified to pH 2 with 5N HCl. The aqueous layer was extracted with EtOAc (2×20 mL). The combined organic extracts were washed with water (30 mL), brine (30 mL), dried over Na₂SO₄, and concentrated to give the desired triazolinone as oil, which was solidified by ether to afford white solid 0.79 g in 88% yield, mp: 157.7° C. ¹H NMR (CD₃OD) δ 7.16 (d, 2H), 7.05 (d, 2H), 6.94 (d, 2H), 6.79 (d, 2H), 4.75 (s, 2H), 2.52 (t, 2H), 2.41 (t, 2H), 2.32 (s, 3H), 1.77 (t, 2H), 1.52 (s, 6H); ¹³C NMR (CD₃OD) δ 173.6, 157.4, 150.7, 145.7, 134.7, 132.5, 130.2, 126.3, 126.2, 125.9, 125.8, 125.7, 123.8, 123.7, 116.5, 75.9, 40.4, 30.7, 24.4, 21.7, 21.4, 16.8. Anal. Calcd for C₂₃H₂₇N₃O₄.½H₂O: C, 66.01; H, 6.74; N, 10.04. Found: C, 66.12; H, 6.71; N, 9.87.

### 2-(4-{3-[4-(3,4,5-Trimethoxy-benzyl)-5-oxo-4, 5-dihydro-1H-[1,2,4]triazol-3-yl]-propyl})-phenoxy)-2-methyl-propionic acid (7f)

To a solution of 2-(4-{N′-3,4,5-trimethoxy-benzylaminocarbonyl)-hydrazino]-4-oxo-butyl}-phenoxy)-2-methyl-propionic acid tert-butyl ester (6f) (1.0 g, 0.0018 mol) in methanol (20 mL) was added solid potassium hydroxide (1.51 g, 0.027 mol) and the reaction mixture heated at reflux for 32 hours. After cooling to ambient temperature, the reaction mixture was diluted with water (15 mL) and EtOAc (30 mL), and then acidified to pH 2 with 5N HCl. The aqueous layer was extracted with EtOAc (2×20 mL). The combined organic extracts were washed with water (30 mL), brine (30 mL), dried over Na₂SO₄, and concentrated to give the desired triazolinone as an oil (0.87 g), which was purified by flash chromatography (95:5 CH₂Cl₂/MeOH) and solidified by pet ether to afford white solid 0.79 g in 91% yield, mp: 77.3° C. ¹H NMR (CDCl₃) δ 10.11 (s, 1H), 6.96 (d, 2H), 6.81 (d, 2H), 6.38 (s, 2H), 4.66 (s, 2H), 3.85 (s, 3H), 3.79 (s, 6H), 2.61 (t, 2H), 2.40 (t, 2H), 1.94 (t, 2H), 1.60 (s, 6H); ¹³C NMR (CD₃OD) δ 173.9, 153.2, 150.7, 145.3, 132.0, 128.2, 126.1, 120.9, 104.3, 101.6, 101.5, 76.5, 57.9, 53.4, 53.3, 41.6, 30.9, 24.3, 22.3, 22.0. Anal. Calcd for C₂₅H₃₁N₃O₇.2H₂O: C, 57.57; H, 5.99; N, 8.05. Found: C, 57.40; H, 6.11; N, 7.94.

### 2-(4-{3-[4-(2-Chloro-benzyl)-5-oxo-4, 5-dihydro-1H-[1,2,4]triazol-3-yl]-propyl})-phenoxy)-2-methyl-propionic acid (7 g)

To a solution of 2-(4-{N′-2-chloro-benzylaminocarbonyl)-hydrazino]-4-oxo-butyl}-phenoxy)-2-methyl-propionic acid tert-butyl ester (6 g) (1.85 g, 0.0037 mol) in methanol (40 mL) was added solid potassium hydroxide (3.14 g, 0.056 mol) and the reaction mixture heated at reflux for 32 hours. After cooling to ambient temperature, the reaction mixture was diluted with water (20 mL) and EtOAc (35 mL), and then acidified to pH 2 with 5N HCl. The aqueous layer was extracted with ETOAc (2×25 mL). The combined organic extracts were washed with water (35 mL), brine (35 mL), dried over Na₂SO₄, and concentrated to give the desired triazolinone as oil, which was purified by flash chromatography (95:5 CH₂Cl₂/MeOH) afforded 7 g (1.35 g, 84%) as a white solid. mp: 45.1° C. ¹H NMR (CDCl₃) δ 9.80 (s, 1H), 7.40 (d, 1H), 7.22 (m, 2H), 7.06 (d, 1H), 6.96 (d, 2H), 6.82 (d, 2H), 4.90 (s, 2H), 2.59 (t, 2H), 2.39 (t, 2H), 1.91 (t, 2H), 1.60 (s, 6H); ¹³C NMR (CDCl₃) δ 173.7, 153.2, 150.1, 145.4, 132.3, 130.0, 129.6, 126.9, 126.5, 126.2, 125.3, 124.6, 117.2, 76.7, 38.6, 31.0, 24.1, 22.3, 22.2, 18.1. Anal. Calcd for C₂₂H₂₄ClN₃O₄: C, 61.46; H, 5.63; Cl, 8.25; N, 9.77. Found: C, 61.12; H, 5.71; Cl, 8.55; N, 9.87.

### 2-(4-{3-[4-(4-Methoxy-benzyl)-5-oxo-4, 5-dihydro-1H-[1,2,4]triazol-3-yl]-propyl})-phenoxy)-2-methyl-propionic acid methyl ester (8a)

To a solution of crude 2-(4-{3-[4-(4-Methoxy-benzyl)-5-oxo-4, 5-dihydro-1H-[1,2,4]triazol-3-yl]-propyl}-phenoxy)-2-methyl-propionic acid (7a) (4.15 g, 0.01 mol) in methanol (200 mL) was added concentrated sulfuric acid (2 mL) and the reaction mixture stirred at ambient temperature overnight. After concentration to 20% of original volume, the solution was diluted with ETOAc (100 mL), washed with water (100 mL), saturated aqueous NaHCO₃ (2×50 mL), and brine (50 mL). The organic layer was dried over Na₂SO₄, and concentrated to give the methyl ester as a white solid 4.07 g, Crystallization by EtOAc gave white crystals 3.86 g in 88% yield, mp: 130.4° C. ¹H NMR (CDCl₃) δ 8.95 (s, 1H), 7.10 (d, 2H), 6.96 (d, 2H), 6.85 (d, 2H), 6.75 (d, 2H), 4.67 (s, 2H), 3.79 (s, 3H), 3.76 (s, 3H), 2.57 (t, 2H), 2.36 (t, 2H), 1.88 (t, 2H), 1.57 (s, 6H); ¹³C NMR (CDCl₃) δ 172.0, 156.5, 152.7, 150.7, 145.4, 131.9, 126.2, 125.9, 125.8, 124.9, 116.5, 111.4, 111.1, 76.2, 52.4, 49.6, 40.9, 31.1, 24.2, 22.4, 22.3. Anal. Calcd for C₂₄H₂₉N₃O₅: C, 65.59; H, 6.65; N, 9.56. Found: C, 65.86; H, 6.80; N, 9.47.

### 2-(4-{3-[4-(3-Methoxy-benzyl)-5-oxo-4, 5-dihydro-1H-[1,2,4]triazol-3-yl]-propyl})-phenoxy)-2-methyl-propionic acid methyl ester (8b)

To a solution of crude 2-(4-{3-[4-(3-Methoxy-benzyl)-5-oxo-4, 5-dihydro-1H-[1,2,4]triazol-3-yl]-propyl}-phenoxy)-2-methyl-propionic acid (7b) (1.30 g, 0.0031 mol) in methanol (40 mL) was added concentrated sulfuric acid (0.5 mL) and the reaction mixture stirred at ambient temperature overnight. After concentration to 20% of original volume, the solution was diluted with EtOAc (50 mL), washed with water (20 mL), saturated aqueous NaHCO₃ (2×20 mL), and brine (20 mL). The organic layer was dried over Na₂SO₄, and concentrated to give the methyl ester as oil. Solidified by ether gave white solid 1.16 g in 88% yield, mp: 88.6° C. ¹H NMR (CDCl₃) δ 10.82 (s, 1H), 7.22 (t, 1H), 6.94 (d, 2H), 6.80 (d, 1H), 6.71 (d, 4H), 4.71 (s, 2H), 3.75 (s, 6H), 2.54 (t, 2H), 2.35 (t, 2H), 1.85 (t, 2H), 1.55 (s, 6H); ¹³C NMR (CDCl₃) δ 172.0, 157.0, 153.4, 150.6, 145.0, 134.4, 132.0, 127.1, 116.5, 116.4, 110.5, 109.9, 76.2, 52.3, 49.6, 41.2, 31.1, 24.2, 22.4, 22.3. Anal. Calcd for C₂₄H₂₉N₃O₅: C, 65.59; H, 6.65; N, 9.56. Found: C, 65.57; H, 6.70; N, 9.56.

### 2-(4-{3-[4-Benzyl-5-oxo-4,5-dihydro-1H-[1,2,4]triazol-3-yl]-propyl}-phenoxy)-2-methyl-propionic acid methyl ester (8c)

To a solution of crude 2-(4-{3-[4-benzyl-5-oxo-4,5-dihydro-1H-[1,2,4]triazol-3-yl]-propyl}-phenoxy)-2-methyl-propionic acid (7c) (0.70 g, 0.0018 mol) in methanol (30 mL) was added concentrated sulfuric acid (0.3 mL) and the reaction mixture stirred at ambient temperature overnight. After concentration to small volume, the solution was diluted with EtOAc (40 mL), washed with water (25 mL), saturated aqueous NaHCO₃ (2×25 mL), and brine (25 mL). The organic layer was dried over Na₂SO₄, and concentrated to give the methyl ester as an oil 0.75 g. Solidified by pet. Ether gave white solid 0.64 g in 86% yield, mp: 89.1° C. ¹H NMR (CDCl₃) δ 9.34 (s, 1H), 7.34 (m, 2H), 7.13 (t, 2H), 6.95 (d, 2H), 6.75 (d, 2H), 4.75 (s, 2H), 3.77 (s, 3H), 2.56 (t, 2H), 2.36 (t, 2H), 1.88 (t, 2H), 1.57 (s, 6H); ¹³C NMR (CDCl₃) δ 172.0, 152.9, 150.7, 145.3, 132.8, 131.9, 126.2, 126.1, 125.2, 124.3, 116.5, 76.2, 49.6, 41.4, 31.1, 24.2, 22.4, 22.3. Anal. Calcd for C₂₃H₂₇N₃O₄: C, 67.46; H, 6.65; N, 10.26. Found: C, 63.37; H, 6.38; N, 10.04.

### 2-(4-{3-[4-(4-Fluoro-benzyl)-5-oxo-4, 5-dihydro-1H-[1,2,4]triazol-3-yl]-propyl}-phenoxy)-2-methyl-propionic acid methyl ester (8d)

To a solution of crude 2-(4-{3-[4-(4-fluoro-benzyl)-5-oxo-4, 5-dihydro-1H-[1,2,4]triazol-3-yl]-propyl}-phenoxy)-2-methyl-propionic acid (7d) (0.43 g, 0.001 mol) in methanol (15 mL) was added concentrated sulfuric acid (0.3 mL) and the reaction mixture stirred at ambient temperature overnight. After concentration to 20% of original volume, the solution was diluted with ETOAc (25 mL), washed with water (15 mL), saturated aqueous NaHCO₃ (2×15 mL), and brine (15 mL). The organic layer was dried over Na₂SO₄, and concentrated to give the methyl ester as an oil 0.43 g. Solidification by ether gave white solid 0.36 g in 84% yield, mp: 119.3° C. ¹H NMR (CDCl₃) δ 9.65 (s, 1H), 7.14 (t, 2H), 7.01 (m, 4H), 6.75 (d, 2H), 4.69 (s, 2H), 3.76 (s, 3H), 2.58 (t, 2H), 2.35 (t, 2H), 1.89 (t, 2H), 1.57 (s, 6H); ¹³C NMR (CDCl₃) δ 171.9, 160.5, 158.5, 152.8, 150.7, 145.1, 131.7, 128.6, 126.3, 126.2, 126.1, 116.4, 113.1, 112.9, 76.2, 49.5, 40.7, 31.0, 24.1, 22.4, 22.2. Anal. Calcd for C₂₃H₂₆FN₃O₄: C, 64.62; H, 6.13; N, 9.83; F, 4.44. Found: C, 64.92; H, 6.37; N, 9.75; F, 4.26.

### 2-(4-{3-[4-(4-Methyl-benzyl)-5-oxo-4, 5-dihydro-1H-[1,2,4]triazol-3-yl]-propyl}-phenoxy)-2-methyl-propionic acid methyl ester (8e)

To a solution of 2-(4-{3-[4-(4-methyl-benzyl)-5-oxo-4, 5-dihydro-1H-[1,2,4]triazol-3-yl]-propyl}-phenoxy)-2-methyl-propionic acid (7e) (0.79 g, 0.002 mol) in methanol (30 mL) was added concentrated sulfuric acid (0.3 mL) and the reaction mixture stirred at ambient temperature overnight. After concentration to 20% of original volume, the solution was diluted with ETOAc (50 mL), washed with water (30 mL), saturated aqueous NaHCO₃ (2×30 mL), and brine (30 mL). The organic layer was dried over Na₂SO₄, and concentrated to give the methyl ester as an oil 0.84 g. Solidification by ether gave white solid 0.71 g in 84% yield, mp: 122.8° C. ¹H NMR (CDCl₃) δ 9.58 (s, 1H), 7.13 (d, 2H), 7.07 (d, 2H), 6.96 (d, 2H), 6.75 (d, 2H), 4.71 (s, 2H), 3.77 (s, 3H), 2.56 (t, 2H), 2.36 (t, 2H), 2.33 (s, 3H), 1.87 (t, 2H), 1.57 (s, 6H); ¹³C NMR (CDCl₃) δ 172.0, 153.0, 150.7, 145.3, 134.9, 132.0, 129.8, 126.7, 126.2, 124.3, 116.5, 76.2, 49.6, 41.2, 31.1, 24.2, 22.4, 22.3, 18.2. Anal. Calcd for C₂₄H₂₉N₃O₄: C, 68.06; H, 6.90; N, 9.92. Found: C, 67.90; H, 6.98; N, 9.55.

### 2-(4-{3-[4-(3,4,5-Trimethoxy-benzyl)-5-oxo-4, 5-dihydro-1H-[1,2,4]triazol-3-yl]-propyl})-phenoxy)-2-methyl-propionic acid methyl ester (8f)

To a solution of 2-(4-{3-[4-(3,4,5-methoxy-benzyl)-5-oxo-4, 5-dihydro-1H-[1,2,4]triazol-3-yl]-propyl}-phenoxy)-2-methyl-propionic acid (7f) (0.87 g, 0.0018 mol) in methanol (30 mL) was added concentrated sulfuric acid (0.3 mL) and the reaction mixture stirred at ambient temperature overnight. After concentration to 20% of original volume, the solution was diluted with EtOAc (50 mL), washed with water (30 mL), saturated aqueous NaHCO₃ (2×30 mL), and brine (30 mL). The organic layer was dried over Na₂SO₄, and concentrated to give the methyl ester, which was purified by flash chromatography (95:5 CH₂Cl₂/MeOH) afforded 8f (0.80 g, 89%) as pale yellow oil. Solidification by pet. ether afforded white solid, mp: 135.4° C. ¹H ¹H NMR (CDCl₃) δ 10.40 (s, 1H), 6.97 (d, 2H), 6.73 (d, 2H), 6.40 (s, 2H), 4.68 (s, 2H), 3.80 (s, 3H), 3.79 (s, 6H), 3.75 (s, 3H), 2.58 (t, 2H), 2.41 (t, 2H), 1.91 (t, 2H), 1.55 (s, 6H); ¹³C NMR (CDCl₃) δ 171.9, 153.2, 150.7, 145.1, 131.9, 128.5, 126.1, 116.6, 101.5, 76.2, 57.9, 53.3, 53.2, 49.5, 41.6, 31.1, 24.1, 22.4, 22.3. Anal. Calcd for C₂₆H₃₃N₃O₇.H₂O: C, 60.33; H, 6.42; N, 8.12. Found: C, 60.55; H, 6.35; N, 8.41.

### 2-(4-{3-[4-(2-Chloro-benzyl)-5-oxo-4, 5-dihydro-1H-[1,2,4]triazol-3-yl]-propyl})-phenoxy)-2-methyl-propionic acid methyl ester (8 g)

To a solution of 2-(4-{3-[4-(2-chloro-benzyl)-5-oxo-4, 5-dihydro-1H-[1,2,4]triazol-3-yl]-propyl}-phenoxy)-2-methyl-propionic acid (7 g) (1.35 g, 0.0031 mol) in methanol (40 mL) was added concentrated sulfuric acid (0.35 mL) and the reaction mixture stirred at ambient temperature overnight. After concentration to 20% of original volume, the solution was diluted with EtOAc (50 mL), washed with water (30 mL), saturated aqueous NaHCO₃ (2×30 mL), and brine (30 mL). The organic layer was dried over Na₂SO₄, and concentrated to give the methyl ester as an oil 1.33 g, which was used without further purification in the next step. ¹H NMR (CDCl₃) δ 10.80 (s, 1H), 7.38 (d, 1H), 7.20 (m, 2H), 7.06 (t, 1H), 6.93 (d, 2H), 6.72 (d, 2H), 4.92 (s, 2H), 3.75 (s, 3H), 2.55 (t, 2H), 2.36 (t, 2H), 1.87 (t, 2H), 1.56 (s, 6H); ¹³C NMR (CDCl₃) δ 172.0, 153.4, 150.6, 145.0, 131.9, 130.3, 129.5, 126.8, 126.3, 126.2, 126.1, 125.1, 124.6, 116.5, 76.2, 50.6, 38.5, 31.1, 24.3, 22.4, 22.2.

### 2-(4-{3-[4-(4-Methoxy-benzyl)-1-(4-methyl-benzyl)-5-oxo-4,5-dihydro-1H-[1,2,4]triazol-3-yl]-propyl}-phenoxy)-2-methyl-propionic acid methyl ester (9a)

To a solution of 2-(4-{3-[4-(4-Methoxy-benzyl)-5-oxo-4,5-dihydro-1H-[1,2,4]triazol-3-yl]-propyl}-phenoxy)-2-methyl-propionic acid methyl ester (8a) (1.16 g, 0.0026 mol) in DMF (10 ml), was added α-bromo-p-xylene (1.15 g, 0.0062 mol) and powdered K₂CO₃ (2.0 g, 0.015 mol) and the resulting mixture heated at 45° C. for overnight. After cooling to ambient temperature, the reaction mixture was diluted with water (100 mL) and extracted with EtOAc (3×30 mL). The combined organic extracts were concentrated to an oil, which was purified by flash chromatography (gradient elution, 8:2 to 1:1 Hexanes/EtOAc to give the desired product as oil 1.41 g. ¹H NMR (CDCl₃) δ 7.24 (d, 2H), 7.13 (d, 2H), 7.10 (d, 2H), 6.93 (d, 2H), 6.83 (d, 2H), 6.74 (d, 2H), 4.91 (s, 2H), 4.68 (s, 2H), 3.78 (s, 3H), 3.76 (s, 3H), 2.52 (t, 2H), 2.34 (t, 2H), 2.31 (s, 3H), 1.83 (t, 2H), 1.57 (s, 6H); ¹³C NMR (CDCl₃) δ 172.0, 157.1, 151.2, 150.7, 143.4, 134.6, 134.5, 132.0, 130.8, 127.1, 126.4, 126.2, 125.2, 116.4, 110.6, 109.8, 76.2, 52.3, 49.6, 46.0, 41.7, 31.2, 24.5, 22.4, 22.3, 18.2.

### 2-(4-{3-[4-(3-Methoxy-benzyl)-1-(4-methyl-benzyl)-5-oxo-4,5-dihydro-1H-[1,2,4]triazol-3-yl]-propyl}-phenoxy)-2-methyl-propionic acid methyl ester (9b)

To a solution of 2-(4-{3-[4-(3-Methoxy-benzyl)-5-oxo-4,5-dihydro-1H-[1,2,4]triazol-3-yl]-propyl}-phenoxy)-2-methyl-propionic acid methyl ester (8b) (0.93 g, 0.0021 mol) in DMF (25 ml), was added α-bromo-p-xylene (0.61 g, 0.0033 mol) and powdered K₂CO₃ (1.52 g, 0.011 mol) and the resulting mixture heated at 45° C. for overnight. After cooling to ambient temperature, the reaction mixture was diluted with water (100 mL) and extracted with ETOAc (3×30 mL). The combined organic extracts were concentrated to an oil, which was purified by flash chromatography (gradient elution, 8:2 to 1:1 Hexanes/ETOAc to give the desired product as oil 1.14 g. ¹H NMR (CDCl₃) δ 7.12 (d, 2H), 6.91 (d, 2H), 6.79 (d, 1H), 6.73 (t, 3H), 4.92 (s, 2H), 4.71 (s, 2H), 3.75 (s, 3H), 3.72 (s, 3H), 2.50 (t, 2H), 2.33 (t, 2H), 2.32 (s, 3H), 1.82 (t, 2H), 1.59 (s, 6H); ¹³C NMR (CDCl₃) δ 173.5, 157.1, 151.6, 150.6, 143.3, 134.5, 134.4, 131.9, 130.8, 127.0, 126.3, 126.2, 126.1, 125.1, 116.4, 110.5, 109.7, 76.1, 52.2, 49.5, 45.9, 41.7, 31.1, 24.5, 22.4, 22.3, 18.2.

### 2-(4-{3-[4-Benzyl-1-(4-methyl-benzyl)-5-oxo-4,5-dihydro-1H-[1,2,4]triazol-3-yl]-propyl}-phenoxy)-2-methyl-propionic acid methyl ester (9c)

To a solution of 2-(4-{3-[4-(3-Methoxy-benzyl)-5-oxo-4,5-dihydro-1H-[1,2,4]triazol-3-yl]-propyl}-phenoxy)-2-methyl-propionic acid methyl ester (8c) (0.55 g, 0.0013 mol) in DMF (10 ml), was added α-bromo-p-xylene (0.60 g, 0.0032 mol) and powdered K₂CO₃ (1.10 g, 0.008 mol) and the resulting mixture heated at 45° C. for overnight. After cooling to ambient temperature, the reaction mixture was diluted with water (50 mL) and extracted with EtOAc (3×30 mL). The combined organic extracts were concentrated to an oil, which was purified by flash chromatography (gradient elution, 9:1 to 7:3 Hexanes/EtOAc to give the desired product as oil 0.59 g in 88% yield. ¹H NMR (CDCl₃) δ 7.29 (m, 4H), 7.16 (t, 1H), 7.13 (m, 4H), 6.92 (d, 2H), 6.74 (d, 2H), 4.93 (s, 2H), 4.75 (s, 2H), 3.76 (s, 3H), 2.51 (t, 2H), 2.33 (t, 2H), 2.30 (s, 3H), 1.83 (t, 2H), 1.57 (s, 6H); ¹³C NMR (CDCl₃) δ 172.0, 151.3, 150.7, 143.4, 134.5, 133.0, 132.0, 130.8, 126.4, 126.2, 125.2, 125.1, 124.3, 116.4, 76.2, 49.6, 46.0, 41.8, 31.2, 24.5, 22.4, 22.3, 18.3.

### 2-(4-{3-[4-(4-Flouoro-benzyl-1-(4-methyl-benzyl)-5-oxo-4,5-dihydro-1H-[1,2,4]triazol-3-yl]-propyl}-phenoxy)-2-methyl-propionic acid methyl ester (9d)

To a solution of 2-(4-{3-[4-(4-fluoro-benzyl)-5-oxo-4,5-dihydro-1H-[1,2,4]triazol-3-yl]-propyl}-phenoxy)-2-methyl-propionic acid methyl ester (8d) (0.36 g, 0.00084 mol) in DMF (5 ml), was added α-bromo-p-xylene (0.31 g, 0.0017 mol) and powdered K₂CO₃ (0.70 g, 0.005 mol) and the resulting mixture heated at 45° C. for overnight. After cooling to ambient temperature, the reaction mixture was diluted with water (50 mL) and extracted with ETOAc (3×30 mL). The combined organic extracts were concentrated to an oil, which was purified by flash chromatography (gradient elution, 8:2 to 1:1 Hexanes/ETOAc to give the desired product as an oil 0.36 g in 78% yield. ¹H NMR (CDCl₃) δ 7.24 (d, 2H), 7.14 (m, 4H), 7.01 (t, 2H), 6.93 (d, 2H), 6.74 (d, 2H), 4.92 (s, 2H), 4.69 (s, 2H), 3.77 (s, 3H), 2.53 (t, 2H), 2.32 (t, 2H), 2.29 (s, 3H), 2.32 (t, 2H), 1.84 (t, 2H), 1.58 (s, 6H); ¹³C NMR (CDCl₃) δ 172.0, 160.4, 158.5, 151.1, 150.7, 143.2, 134.5, 131.8, 130.7, 128.9, 126.4, 126.2, 126.1, 125.2, 116.4, 113.0, 112.9, 76.2, 49.6, 46.0, 41.1, 31.2, 24.6, 22.4, 22.3, 18.2.

### 2-(4-{3-[4-(4-Methyl-benzyl-1-(4-methyl-benzyl)-5-oxo-4,5-dihydro-1H-[1,2,4]triazol-3-yl]-propyl}-phenoxy)-2-methyl-propionic acid methyl ester (9e)

To a solution of 2-(4-{3-[4-(4-methyl-benzyl)-5-oxo-4,5-dihydro-1H-[1,2,4]triazol-3-yl]-propyl}-phenoxy)-2-methyl-propionic acid methyl ester (8e) (0.71 g, 0.0017 mol) in DMF (7 ml), was added α-bromo-p-xylene (0.63 g, 0.0034 mol) and powdered K₂CO₃ (1.38 g, 0.01 mol) and the resulting mixture heated at 45° C. for overnight. After cooling to ambient temperature, the reaction mixture was diluted with water (50 mL) and extracted with ETOAc (3×30 mL). The combined organic extracts were concentrated to an oil, which was purified by flash chromatography (gradient elution, 8:2 to 1:1 Hexanes/ETOAc to give the desired product as an oil 0.72 g in 80% yield. ¹H NMR (CDCl₃) δ 7.30 (d, 2H), 7.18 (m, 4H), 7.06 (d, 2H), 6.94 (d, 2H), 6.75 (d, 2H), 4.94 (s, 2H), 4.79 (s, 2H), 3.78 (s, 3H), 2.52 (t, 2H), 2.35 (s, 6H), 2.33 (t, 2H), 1.82 (t, 2H), 1.59 (s, 6H); ¹³C NMR (CDCl₃) δ 171.9, 151.3, 150.7, 143.4, 134.8, 134.4, 132.0, 130.8, 130.1, 126.6, 126.4, 126.3, 126.2, 125.2, 124.6, 116.5, 76.2, 49.5, 46.0, 41.6, 31.2, 24.6, 22.4, 22.3, 18.2.

### 2-(4-{3-[4-(3,4,5-Trimethoxy-benzyl-1-(4-methyl-benzyl)-5-oxo-4,5-dihydro-1H-[1,2,4]triazol-3-yl]-propyl}-phenoxy)-2-methyl-propionic acid methyl ester (9f)

To a solution of 2-(4-{3-[4-(3,4,5-trimethoxy-benzyl)-5-oxo-4,5-dihydro-1H-[1,2,4]triazol-3-yl]-propyl})-phenoxy)-2-methyl-propionic acid methyl ester (8f) (0.80 g, 0.0016 mol) in DMF (10 ml), was added α-bromo-p-xylene (0.67 g, 0.0036 mol) and powdered K₂CO₃ (1.52 g, 0.011 mol) and the resulting mixture heated at 45° C. for overnight. After cooling to ambient temperature, the reaction mixture was diluted with water (50 mL) and extracted with EtOAc (3×30 mL). The combined organic extracts were concentrated to an oil, which was purified by flash chromatography (gradient elution, 9:1 to 7:3 Hexanes/EtOAc to give the desired product as an oil 0.76 g in 78% yield. ¹H NMR (CDCl₃) δ 7.23 (d, 2H), 7.10 (d, 2H), 6.93 (d, 2H), 6.72 (d, 2H), 6.34 (s, 2H), 4.91 (s, 2H), 4.66 (s, 2H), 3.79 (s, 3H), 3.74 (s, 9H), 2.52 (t, 2H), 2.36 (t, 2H), 2.29 (s, 3H), 1.85 (t, 2H), 1.54 (s, 6H); ¹³C NMR (CDCl₃) δ 171.8, 159.6, 151.2, 150.7, 143.3, 134.5, 132.0, 130.8, 128.7, 126.3, 126.1, 125.1, 116.5, 101.3, 76.2, 57.9, 53.2, 49.5, 46.0, 41.9, 31.2, 28.5, 24.5, 22.4, 18.2.

### 2-(4-{3-[4-(2-Chloro-benzyl-1-(4-methyl-benzyl)-5-oxo-4,5-dihydro-1H-[1,2,4]triazol-3-yl]-propyl}-phenoxy)-2-methyl-propionic acid methyl ester (9 g)

To a solution of 2-(4-{3-[4-(2-chloro-benzyl)-5-oxo-4,5-dihydro-1H-[1,2,4]triazol-3-yl]-propyl}-phenoxy)-2-methyl-propionic acid methyl ester (8 g) (1.33 g, 0.003 mol) in DMF (10 ml), was added α-bromo-p-xylene (1.25 g, 0.0068 mol) and powdered K₂CO₃ (2.50 g, 0.018 mol) and the resulting mixture heated at 45° C. for overnight. After cooling to ambient temperature, the reaction mixture was diluted with water (50 mL) and extracted with EtOAc (3×30 mL). The combined organic extracts were concentrated to an oil, which was purified by flash chromatography (gradient elution, 9:1, 8:2 to 7:3 Hexanes/EtOAc to give the desired product as an oil 1.45 g in 88% yield. ¹H NMR (CDCl₃) δ 7.40 (dd, 1H), 7.30 (m, 2H), 7.24 (m, 2H), 7.18 (d, 2H), 7.07 (dd, 1H), 6.94 (d, 2H), 6.74 (d, 2H), 4.97 (s, 2H), 4.94 (s, 2H), 3.79 (s, 3H), 2.53 (t, 2H), 2.36 (t, 2H), 2.34 (s, 3H), 1.83 (t, 2H), 1.59 (s, 6H); ¹³C NMR (CDCl₃) δ 172.0, 151.2, 150.6, 143.4, 134.5, 131.9, 130.7, 130.4, 129.5, 126.8, 126.4, 126.3, 126.2, 125.3, 125.2, 124.5, 116.4, 76.2, 49.6, 46.0, 39.0, 31.2, 24.6, 22.4, 22.3, 18.3.

### 2-(4-{3-[4-(4-Methyl-benzyl-1-(4-vinyl-benzyl)-5-oxo-4,5-dihydro-1H-[1,2,4]triazol-3-yl]-propyl}-phenoxy)-2-methyl-propionic acid methyl ester (9 h)

To a solution of 2-(4-{3-[4-(4-methyl-benzyl)-5-oxo-4,5-dihydro-1H-[1,2,4]triazol-3-yl]-propyl}-phenoxy)-2-methyl-propionic acid methyl ester (8e) (1.02 g, 0.0024 mol) in DMF (10 ml), was added 4-vinylbenzyl chloride (0.92 g, 0.006 mol) and powdered K₂CO₃ (1.9 g, 0.014 mol) and the resulting mixture heated at 45° C. for overnight. After cooling to ambient temperature, the reaction mixture was diluted with water (50 mL) and extracted with EtOAc (3×30 mL). The combined organic extracts were concentrated to an oil, which was purified by flash chromatography (gradient elution, 9:1 to 7:3 Hexanes/EtOAc to give the desired product as white solid 1.3 g. Crystallization by EtOAc gave white crystals 0.91 g in 70% yield, mp: 76.7° C. ¹H NMR (CDCl₃) δ 7.35 (d, 2H), 7.27 (d, 2H), 7.03 (d, 2H), 7.10 (d, 2H), 6.88 (d, 2H), 6.71 (d, 2H), 6.63 (m, 1H), 5.71 (d, 1H), 5.21 (d, 1H), 4.92 (s, 2H), 4.69 (s, 2H), 3.73 (s, 3H), 2.48 (t, 2H), 2.32 (s, 3H), 2.29 (t, 2H), 1.77 (t, 2H), 1.54 (s, 6H); ¹³C NMR (CDCl₃) δ 172.0, 151.3, 150.7, 143.5, 134.9, 134.2, 133.5, 133.3, 132.0, 130.0, 126.7, 126.2, 125.4, 124.3, 123.5, 116.4, 76.2, 49.6, 45.9, 41.7, 31.2, 24.6, 22.4, 18.2. Anal. Calcd for C₃₃H₃₇N₃O₄: C, 73.44; H, 6.91; N, 7.79. Found: C, 73.75; H, 7.01; N, 7.77.

### 2-(4-{3-[4-(4-Methoxy-benzyl)-1-(3-methoxy-benzyl)-5-oxo-4,5-dihydro-1H-[1,2,4]triazol-3-yl]-propyl}-phenoxy)-2-methyl-propionic acid methyl ester (9i)

To a solution of 2-(4-{3-[4-(4-Methoxy-benzyl)-5-oxo-4,5-dihydro-1H-[1,2,4]triazol-3-yl]-propyl}-phenoxy)-2-methyl-propionic acid methyl ester (8a) (0.33 g, 0.0008 mol) in DMF (10 ml), was added 3-methoxybenzyl bromide (0.40 g, 0.002 mol) and powdered K₂CO₃ (0.7 g, 0.005 mol) and the resulting mixture heated at 45° C. for overnight. After cooling to ambient temperature, the reaction mixture was diluted with water (50 mL) and extracted with EtOAc (3×25 mL). The combined organic extracts were concentrated to an oil, which was purified by flash chromatography (gradient elution, 8:2 to 1:1 Hexanes/EtOAc to give the desired product as oil 0.34 g in 76% yield. ¹H NMR (CDCl₃) δ 7.23 (t, 1H), 7.09 (d, 2H), 6.93 (d, 4H), 6.83 (s, 1H), 6.81 (d, 2H), 6.73 (d, 2H), 4.93 (s, 2H), 4.68 (s, 2H), 3.77 (s, 3H), 3.76 (s, 3H), 3.75 (s, 3H), 2.52 (t, 2H), 2.33 (t, 2H), 1.83 (m, 2H), 1.56 (s, 6H); ¹³C NMR (CDCl₃) δ 171.9, 156.9, 151.3, 150.7, 143.5, 134.6, 135.3, 132.0, 126.7, 126.2, 125.8, 125.1, 117.3, 116.4, 111.3, 110.5, 110.4, 76.2, 52.4, 52.3, 49.5, 46.1, 41.4, 31.2, 24.6, 22.4, 22.3.

### 2-(4-{3-[4-(4-Methoxy-benzyl)-1-(4-t-butyl-benzyl)-5-oxo-4,5-dihydro-1H-[1,2,4]triazol-3-yl]-propyl}-phenoxy)-2-methyl-propionic acid methyl ester (9j)

To a solution of 2-(4-{3-[4-(4-Methoxy-benzyl)-5-oxo-4,5-dihydro-1H-[1,2,4]triazol-3-yl]-propyl}-phenoxy)-2-methyl-propionic acid methyl ester (8a) (0.33 g, 0.0008 mol) in DMF (10 ml), was added 3-methoxybenzyl bromide (0.40 g, 0.002 mol) and powdered K₂CO₃ (0.7 g, 0.005 mol) and the resulting mixture heated at 45° C. for overnight. After cooling to ambient temperature, the reaction mixture was diluted with water (50 mL) and extracted with EtOAc (3×25 mL). The combined organic extracts were concentrated to an oil, which was purified by flash chromatography (gradient elution, 8:2 to 1:1 Hexanes/EtOAc to give the desired product as oil 0.30 g in 64% yield. ¹H NMR (CDCl₃) δ 7.34 (d, 2H), 7.28 (d, 2H), 7.10 (d, 2H), 6.94 (d, 2H), 6.84 (d, 2H), 6.75 (d, 2H), 4.92 (s, 2H), 4.68 (s, 2H), 3.79 (s, 3H), 3.76 (s, 3H), 2.52 (t, 2H), 2.35 (t, 2H), 1.82 (t, 2H), 1.57 (s, 6H), 1.32 (s, 9H); ¹³C NMR (CDCl₃) δ 172.0, 156.4, 151.3, 150.7, 147.6, 143.4, 132.0, 130.7, 126.2, 125.8, 125.1, 124.9, 122.6, 116.4, 111.3, 76.2, 52.4, 50.0, 45.8, 41.4, 31.6, 28.4, 24.6, 22.4, 22.3.

### 2-(4-{3-[4-(4-Methoxy-benzyl)-1-(4-trifluoromethyl-benzyl)-5-oxo-4,5-dihydro-1H-[1,2,4]triazol-3-yl]-propyl}-phenoxy)-2-methyl-propionic acid methyl ester (9k)

To a solution of 2-(4-{3-[4-(4-Methoxy-benzyl)-5-oxo-4,5-dihydro-1H-[1,2,4]triazol-3-yl]-propyl}-phenoxy)-2-methyl-propionic acid methyl ester (8a) (0.51 g, 0.00012 mol) in DMF (10 ml), was added 4-(trifluromethyl)benzyl bromide (0.80 g, 0.0033 mol) and powdered K₂CO₃ (1.1 g, 0.008 mol) and the resulting mixture heated at 45° C. for overnight. After cooling to ambient temperature, the reaction mixture was diluted with water (50 mL) and extracted with EtOAc (3×25 mL). The combined organic extracts were concentrated to an oil, which was purified by flash chromatography (gradient elution, 8:2 to 1:1 Hexanes/EtOAc to give the desired product as oil 0.65 g in 90% yield. ¹H NMR (CDCl₃) δ 7.59 (d, 2H), 7.45 (d, 2H), 7.10 (d, 2H), 6.94 (d, 2H), 6.85 (d, 2H), 6.75 (d, 2H), 5.00 (s, 2H), 4.69 (s, 2H), 3.79 (s, 3H), 3.76 (s, 3H), 2.54 (t, 2H), 2.36 (t, 2H), 1.84 (t, 2H), 1.57 (s, 6H); ¹³C NMR (CDCl₃) δ 171.9, 156.5, 151.3, 150.7, 143.9, 137.7, 131.8, 127.2, 126.9, 126.2, 125.8, 124.9, 122.8, 122.7, 122.6, 122.3, 116.5, 111.4, 76.2, 52.4, 49.5, 45.6, 41.5, 31.2, 24.5, 22.4, 22.3.

### 2-(4-{3-[4-(4-Methoxy-benzyl)-1-(4-trifluoromethoxy-benzyl)-5-oxo-4,5-dihydro-1H-[1,2,4]triazol-3-yl]-propyl}-phenoxy)-2-methyl-propionic acid methyl ester (91)

To a solution of 2-(4-{3-[4-(4-Methoxy-benzyl)-5-oxo-4,5-dihydro-1H-[1,2,4]triazol-3-yl]-propyl}-phenoxy)-2-methyl-propionic acid methyl ester (8a) (0.33 g, 0.0008 mol) in DMF (10 ml), was added 3-methoxybenzyl bromide (0.40 g, 0.002 mol) and powdered K₂CO₃ (0.7 g, 0.005 mol) and the resulting mixture heated at 45° C. for overnight. After cooling to ambient temperature, the reaction mixture was diluted with water (50 mL) and extracted with EtOAc (3×25 mL). The combined organic extracts were concentrated to an oil, which was purified by flash chromatography (gradient elution, 8:2 to 1:1 Hexanes/EtOAc to give the desired product as oil 0.30 g in 64% yield. ¹H NMR (CDCl₃) δ 7.38 (d, 2H), 7.36 (d, 2H), 7.09 (d, 2H), 6.94 (d, 2H), 6.85 (d, 2H), 6.74 (d, 2H), 4.95 (s, 2H), 4.68 (s, 2H), 3.79 (s, 3H), 3.76 (s, 3H), 2.53 (t, 2H), 2.36 (t, 2H), 1.83 (t, 2H), 1.57 (s, 6H); ¹³C NMR (CDCl₃) δ 172.0, 156.5, 151.2, 150.7, 145.8, 143.8, 132.5, 131.9, 126.6, 125.8, 125.0, 118.2, 116.5, 111.4, 76.2, 52.4, 49.5, 45.4, 41.4, 31.2, 24.5, 22.4.

### 2-(4-{3-[4-(4-Methoxy-benzyl)-1-(4-methyl-benzyl)-5-oxo-4,5-dihydro-1H[1,2,4]triazol-3-yl]-propyl}-phenoxy)-2-methyl-propionic acid (10a)

A solution of 2-(4-{3-[4-(4-Methoxy-benzyl)-1-(4-methyl-benzyl)-5-oxo-4,5-dihydro-1H[1,2,4]triazol-3-yl]-propyl}-phenoxy)-2-methyl-propionic acid methyl ester (9a) (1.41 g, 0.0026 mol) in methanol (10 mL) was treated with 2N NaOH (5.0 ml) and the mixture stirred overnight at ambient temperature. After concentration to dryness, the residue was dissolved in water (35 mL), the solution acidified to pH 3 with concentrated hydrochloric acid, then extracted into methylene chloride (3×20 mL). The combined organic extracts were dried over Na₂SO₄, and then concentrated in vacuo to provide the carboxylic acid as waxy oil. Solidification of the waxy oil from 9:1 ether/pet ether gave white solid (1a) 1.03 g in 75% yield, mp: 118.6° C. ¹H NMR (CDCl₃) δ 7.22 (d, 2H), 7.16 (d, 2H), 7.09 (d, 2H), 6.90 (d, 2H), 6.83 (d, 2H), 6.79 (d, 2H), 4.93 (s, 2H), 4.77 (s, 2H), 3.80 (s, 3H), 2.53 (t, 2H), 2.34 (s, 3H), 2.31 (t, 2H), 1.84 (t, 2H), 1.60 (s, 6H); ¹³C NMR (CDCl₃) δ 177.7, 160.6, 155.5, 154.3, 147.8, 138.8, 136.9, 134.9, 130.7, 130.6, 130.1, 129.5, 129.3, 121.6, 115.7, 67.2, 56.7, 50.3, 45.7, 35.4, 28.6, 26.6, 22.5. Anal. Calcd for C₃₁H₃₅N₃O₅.H₂O: C, 67.98; H, 6.81; N, 7.67. Found: C, 67.83; H, 6.69; N, 7.48.

### 2-(4-{3-[4-(3-Methoxy-benzyl)-1-(4-methyl-benzyl)-5-oxo-4,5-dihydro-1H[1,2,4]triazol-3-yl]-propyl}-phenoxy)-2-methyl-propionic acid (10b)

A solution of 2-(4-{3-[4-(3-Methoxy-benzyl)-1-(4-methyl-benzyl)-5-oxo-4,5-dihydro-1H[1,2,4]triazol-3-yl]-propyl})-phenoxy)-2-methyl-propionic acid methyl ester (9b) (1.14 g, 0.0021 mol) in methanol (10 mL) was treated with 2N NaOH (5.0 mL) and the mixture stirred overnight at ambient temperature. After concentration to dryness, the residue was dissolved in CH₂Cl₂ (10 mL), water (35 mL) was added, and the solution was acidified to pH3 with concentrated hydrochloric acid, then extracted into methylene chloride (3×20 mL). The combined organic extracts were dried over Na₂SO₄, and then concentrated in vacuo to provide the carboxylic acid as waxy oil. Solidification of the waxy oil from 9:1 ether/pet ether gave white solid (1) 0.72 g in 53% yield, mp: 117.6° C. ¹H NMR (CDCl₃) δ 7.24 (d, 2H), 7.21 (t, 1H), 7.13 (d, 3H), 6.89 (d, 2H), 6.81 (t, 2H), 6.70 (d, 2H), 4.92 (s, 2H), 4.64 (s, 2H), 3.76 (s, 3H), 2.52 (t, 2H), 2.32 (s, 3H), 2.29 (t, 2H), 1.85 (t, 2H), 1.56 (s, 6H); ¹³C NMR (CDCl₃) δ 173.3, 158.5, 152.5, 149.8, 143.8, 134.5, 134.4, 132.6, 130.6, 127.0, 126.3, 126.2, 126.1, 125.2, 117.3, 116.4, 110.5, 109.8, 76.8, 52.3, 46.0, 41.7, 31.1, 24.3, 22.2, 22.1. Anal. Calcd for C₃₁H₃₅N₃O₅. ½H₂O: C, 69.12; H, 6.54; N, 7.80. Found: C, 68.91; H, 6.47; N, 7.86.

### 2-(4-{3-[4-Benzyl-1-(4-methyl-benzyl)-5-oxo-4,5-dihydro-1H[1,2,4]triazol-3-yl]-propyl}-phenoxy)-2-methyl-propionic acid (10c)

A solution of 2-(4-{3-[4-benzyl-1-(4-methyl-benzyl)-5-oxo-4, 5-dihydro-1H-[1,2,4]triazol-3-yl]-propyl}-phenoxy)-2-methyl-propionic acid methyl ester (9c) (0.55 g, 0.001 mol) in methanol (7.5 mL) was treated with 2N NaOH (5 mL) and the mixture stirred overnight at ambient temperature. After concentration to dryness, the residue was dissolved in CH₂Cl₂ (25 mL), water (5 mL) was added, and the solution was acidified to pH3 with concentrated hydrochloric acid, then extracted into methylene chloride (3×20 mL). The combined organic extracts were dried over Na₂SO₄, and then concentrated in vacuo to provide the carboxylic acid as waxy oil, which was purified by flash chromatography (95:5 CH₂Cl₂/MeOH to give the desired product as an oil (1c) 0.39 g in 78% yield. Solidification of the waxy oil from 9:1 ether/pet ether gave white solid, mp: 49.8° C. ¹H NMR (CDCl₃) δ 7.24 (m, 4H), 7.11 (t, 4H), 6.79 (dd, 4H), 4.92 (s, 2H), 4.74 (s, 2H), 2.43 (t, 2H), 2.30 (s, 3H), 2.29 (t, 2H), 1.76 (t, 2H), 1.47 (s, 6H); ¹³C NMR (CDCl₃) δ 175.5, 151.5, 143.4, 134.4, 132.9, 132.1, 127.0, 126.9, 126.3, 125.9, 125.2, 125.0, 124.7, 124.2, 124.0, 117.4, 116.8, 76.9, 45.9, 41.8, 31.1, 26.8, 22.6, 21.6, 18.2. Anal. Calcd for C₃₀H₃₃N₃O₄.2H₂O: C, 65.08; H, 6.37; N, 7.59. Found: C, 65.02; H, 6.28; N, 7.49.

### 2-(4-{3-[4-Fluoro-benzyl-1-(4-methyl-benzyl)-5-oxo-4,5-dihydro-1H[1,2,4]triazol-3-yl]-propyl}-phenoxy)-2-methyl-propionic acid (10d)

A solution of 2-(4-{3-[4-(4-fluoro-benzyl)-1-(4-methyl-benzyl)-5-oxo-4,5-dihydro-1H[1,2,4]triazol-3-yl]-propyl}-phenoxy)-2-methyl-propionic acid methyl ester (9d) (0.36 g, 0.00068 mol) in methanol (7.5 mL) was treated with 2N NaOH (5 mL) and the mixture stirred overnight at ambient temperature. After concentration to dryness, the residue was dissolved in CH₂Cl₂ (30 mL), water (10 mL) was added, and the solution was acidified to pH3 with concentrated hydrochloric acid, then extracted into methylene chloride (2×30 mL). The combined organic extracts were dried over Na₂SO₄, and then concentrated in vacuo to provide the carboxylic acid as waxy oil. Solidification of the waxy oil from methylene chloride gave white solid (10d) 0.28 g in 80% yield, mp: 60.2° C. ¹H NMR (CDCl₃) δ 7.23 (d, 2H), 7.11 (t, 4H), 6.97 (t, 2H), 6.83 (m, 4H), 4.88 (s, 2H), 4.62 (s, 2H), 2.48 (t, 2H), 2.29 (s, 3H), 2.30 (t, 2H), 1.80 (t, 2H), 1.57 (s, 6H); ¹³C NMR (CDCl₃) δ 170.7, 160.4, 158.5, 156.4, 151.0, 143.2, 134.6, 130.6, 128.8, 126.4, 126.2, 126.1, 125.9, 125.3, 117.7, 113.0, 112.8, 76.7, 46.0, 41.1, 31.1, 24.4, 22.3, 22.2, 18.2. Anal. Calcd for C₃₀H₃₂FN₃O₄.⅔H₂O: C, 68.03; H, 6.34; N, 7.93; F, 3.58. Found: C, 67.83; H, 6.43; N, 7.72; F, 3.23.

### 2-(4-{3-[4-Methyl-benzyl-1-(4-methyl-benzyl)-5-oxo-4,5-dihydro-1H[1,2,4]triazol-3-yl]-propyl}-phenoxy)-2-methyl-propionic acid (10e)

A solution of 2-(4-{3-[4-(4-methyl-benzyl)-1-(4-methyl-benzyl)-5-oxo-4,5-dihydro-1H[1,2,4]triazol-3-yl]-propyl}-phenoxy)-2-methyl-propionic acid methyl ester (9e) (0.72 g, 0.0014 mol) in methanol (15 mL) was treated with 2N NaOH (10 mL) and the mixture stirred overnight at ambient temperature. After concentration to dryness, the residue was dissolved in CH₂Cl₂ (30 mL), water (10 mL) was added, and the solution was acidified to pH3 with concentrated hydrochloric acid, then extracted into methylene chloride (2×30 mL). The combined organic extracts were dried over Na₂SO₄, and then concentrated in vacuo to provide the carboxylic acid as an oil, which was purified by flash chromatography (98:2 CH₂Cl₂/MeOH to give the desired product as a waxy oil 0.57 g in 78% yield. ¹H NMR (CDCl₃) δ 7.24 (t, 2H), 7.12 (m, 4H), 7.07 (d, 2H), 6.83 (dd, 4H), 4.90 (s, 2H), 4.65 (s, 2H), 2.46 (t, 2H), 2.30 (s, 6H), 2.29 (t, 2H), 1.77 (t, 2H), 1.51 (s, 6H); ¹³C NMR (CDCl₃) δ 174.6, 151.2, 150.2, 143.5, 134.9, 134.8, 132.5, 126.8, 126.6, 126.3, 125.8, 125.4, 125.2, 124.9, 124.5, 124.3, 117.6, 77.1, 50.5, 46.0, 41.7, 31.2, 26.8, 24.4, 22.3, 18.2. Anal. Calcd for C₃₁H₃₅N₃O₄ 2H₂O: C, 67.73; H, 6.42; N, 7.65. Found: C, 67.83; H, 6.45; N, 7.42.

### 2-(4-{3-[3,4,5-Trimethoxy-benzyl-1-(4-methyl-benzyl)-5-oxo-4,5-dihydro-1H[1,2,4]triazol-3-yl]-propyl}-phenoxy)-2-methyl-propionic acid (10f)

A solution of 2-(4-{3-[4-(3,4,5-trimethoxy-benzyl)-1-(4-methyl-benzyl)-5-oxo-4,5-dihydro-1H[1,2,4]triazol-3-yl]-propyl}-phenoxy)-2-methyl-propionic acid methyl ester (9f) (0.76 g, 0.0013 mol) in methanol (15 mL) was treated with 2N NaOH (10 mL) and the mixture stirred five hours at ambient temperature. After concentration to dryness, the residue was dissolved in CH₂Cl₂ (35 mL), water (10 mL) was added, and the solution was acidified to pH2 with concentrated hydrochloric acid, then extracted into methylene chloride (3×30 mL). The combined organic extracts were dried over Na₂SO₄, and then concentrated in vacuo to provide the carboxylic acid as waxy oil, which was purified by flash chromatography (98:2 CH₂Cl₂/MeOH) to give the desired product which was solidified from pet. ether to give white solid (10f) 0.57 g in 74% yield, mp: 58.8° C. ¹H NMR (CDCl₃) δ 7.27 (d, 2H), 7.12 (d, 2H), 6.90 (d, 2H), 6.78 (d, 2H), 6.31 (s, 2H), 4.94 (s, 2H), 4.64 (s, 2H), 3.80 (s, 3H), 3.74 (s, 6H), 2.56 (t, 2H), 2.34 (t, 2H), 2.31 (s, 3H), 1.88 (t, 2H), 1.56 (s, 6H); ¹³C NMR (CDCl₃) δ 173.4, 151.3, 150.6, 150.0, 143.5, 134.6, 132.4, 130.7, 128.6, 126.4, 126.2, 125.2, 117.3, 101.2, 76.7, 58.0, 53.2, 46.1, 41.9, 31.0, 24.1, 22.2, 22.1, 18.2. Anal. Calcd for C₃₃H₃₉N₃O₇.½H₂O: C, 66.19; H, 6.56; N, 7.02. Found: C, 65.98; H, 6.27; N, 6.81.

### 2-(4-{3-[2-Chloro-benzyl-1-(4-methyl-benzyl)-5-oxo-4,5-dihydro-11H[1,2,4]triazol-3-yl]-propyl}-phenoxy)-2-methyl-propionic acid (10 g)

A solution of 2-(4-{3-[4-(2-chloro-benzyl)-1-(4-methyl-benzyl)-5-oxo-4,5-dihydro-1H[1,2,4]triazol-3-yl]-propyl}-phenoxy)-2-methyl-propionic acid methyl ester (9 g) (1.45 g, 0.0026 mol) in methanol (15 mL) was treated with 2N NaOH (10 mL) and the mixture stirred overnight at ambient temperature. The reaction mixture was concentrated, dissolved in CH₂Cl₂ (30 mL), water (10 mL) was added, and the solution was acidified to pH3 with concentrated hydrochloric acid, then extracted into methylene chloride (2×30 mL). The combined organic extracts were dried over Na₂SO₄, and then concentrated in vacuo to provide the carboxylic acid as waxy oil. Solidification of the waxy oil from pet.ether gave white solid (10 g) 1.02 g in 73% yield, mp: 126.5° C. ¹H NMR (CDCl₃) δ 7.36 (t, 1H), 7.27 (t, 1H), 7.20 (m, 2H), 7.14 (d, 2H), 7.03 (d, 2H), 6.90 (d, 2H), 6.79 (d, 2H), 4.94 (s, 2H), 4.88 (s, 2H), 2.52 (t, 2H), 2.34 (s, 3H), 2.31 (t, 2H), 1.82 (t, 2H), 1.56 (s, 6H); ¹³C NMR (CDCl₃) δ 173.9, 151.3, 149.9, 143.5, 134.6, 132.6, 130.5, 130.3, 129.5, 126.8, 126.4, 126.2, 125.4, 125.3, 124.6, 117.4, 76.7, 46.1, 39.0, 31.2, 24.5, 22.3, 22.2, 18.3. Anal. Calcd for C₃₀H₃₂ClN₃O₄: C, 67.47; H, 6.04; N, 7.87; Cl, 6.64. Found: C, 67.65; H, 5.90; N, 6.79, Cl, 6.93.

### 2-(4-{3-[4-(4-Methyl-benzyl-1-(4-vinyl-benzyl)-5-oxo-4,5-dihydro-1H-[1,2,4]triazol-3-yl]-propyl}-phenoxy)-2-methyl-propionic acid (10 h)

To a solution of 2-(4-{3-[4-(4-Methyl-benzyl-1-(4-vinyl-benzyl)-5-oxo-4,5-dihydro-1H-[1,2,4]triazol-3-yl]-propyl}-phenoxy)-2-methyl-propionic acid methyl ester (9 h) (0.33 g, 0.0006 mol) in methanol (7.5 mL) was treated with 2N NaOH (5 mL) and the mixture stirred 48 hours at ambient temperature. The reaction mixture was concentrated, dissolved in CH₂Cl₂ (10 mL), water (5 mL) was added, and the solution was acidified to pH3 with concentrated hydrochloric acid, then extracted into methylene chloride (2×20 mL). The combined organic extracts were dried over Na₂SO₄, and then concentrated in vacuo to provide the carboxylic acid as waxy oil. Solidification of the waxy oil from ether gave white solid (10 h) 0.25 g in 78% yield, mp: 127.4° C. ¹H NMR (CDCl₃) δ 7.37 (d, 2H), 7.32 (d, 2H), 7.11 (d, 2H), 7.04 (d, 2H), 6.90 (d, 2H), 6.81 (d, 2H), 6.71 (m, 1H), 5.73 (d, 1H), 5.23 (d, 1H), 4.95 (s, 2H), 4.68 (s, 2H), 2.51 (t, 2H), 2.34 (t, 2H), 2.31 (s, 3H), 1.82 (t, 2H), 1.57 (s, 6H); ¹³C NMR (CDCl₃) δ 173.8, 151.3, 150.0, 143.7, 134.9, 134.2, 133.5, 133.1, 132.6, 129.8, 126.7, 126.3, 125.5, 124.3, 123.6, 117.3, 111.1, 76.7, 46.0, 41.7, 41.7, 31.2, 24.4, 22.4, 22.2, 18.2. Anal. Calcd for C₃₂H₃₅N₃O₄: C, 73.12; H, 6.71; N, 7.99. Found: C, 72.85; H, 6.71; N, 7.72.

### 2-(4-{3-[4-(4-Methoxy-benzyl)-1-(3-methoxy-benzyl)-5-oxo-4,5-dihydro-1H[1,2,4]triazol-3-yl]-propyl}-phenoxy)-2-methyl-propionic acid (10i)

A solution of 2-(4-{3-[4-(4-Methoxy-benzyl)-1-(3-methoxy-benzyl)-5-oxo-4,5-dihydro-1H[1,2,4]triazol-3-yl]-propyl}-phenoxy)-2-methyl-propionic acid methyl ester (9i) (0.25 g, 0.00045 mol) in methanol (10 mL) was treated with 2N NaOH (7 ml) and the mixture stirred 4 hours at ambient temperature. After concentration to dryness, the residue was dissolved in CH₂Cl₂ (35 mL), water (10 mL), the solution acidified to pH 2 with concentrated hydrochloric acid, then extracted into methylene chloride (3×20 mL). The combined organic extracts were dried over Na₂SO₄, and then concentrated in vacuo to provide the carboxylic acid as waxy oil, which was purified by flash chromatography (95:5 CH₂Cl₂/MeOH) to give the desired product which was solidified from pet. ether to give white solid (10i) 0.19 g in 76% yield, mp: 58.8° C.? ¹H NMR (CDCl₃) δ 7.25 (m, 1H), 7.08 (d, 2H), 6.93 (m, 3H), 6.82 (m, 4H), 6.83 (d, 2H), 4.92 (s, 2H), 4.63 (s, 2H), 3.78 (s, 3H), 3.77 (s, 3H), 2.54 (t, 2H), 2.34 (t, 2H), 1.87 (t, 2H), 1.55 (s, 6H); ¹³C NMR (CDCl₃) δ 168.3, 156.9, 156.3, 143.5, 135.2, 132.8, 126.7, 126.3, 125.8, 125.1, 117.6, 111.4, 110.5, 76.1, 57.5, 52.4, 46.1, 31.1, 24.2, 22.3, 22.1. Anal. Calcd for C₃₁H₃₅N₃O₆.3/2H₂O: C, 65.01; H, 6.16; N, 7.33. Found: C, 64.77; H, 6.37; N, 7.17.

### 2-(4-{3-[4-(4-Methoxy-benzyl)-1-(4-t-butyl-benzyl)-5-oxo-4,5-dihydro-1H[1,2,4]triazol-3-yl]-propyl}-phenoxy)-2-methyl-propionic acid (10j)

A solution of 2-(4-{3-[4-(4-Methoxy-benzyl)-1-(4-t-butyl-benzyl)-5-oxo-4,5-dihydro-1H[1,2,4]triazol-3-yl]-propyl}-phenoxy)-2-methyl-propionic acid methyl ester (9j) (0.2 g, 0.00034 mol) in methanol (10 mL) was treated with 2N NaOH (7 ml) and the mixture stirred overnight at ambient temperature. After concentration to dryness, the residue was dissolved in methylene chloride (35 ml), water (10 mL), the solution acidified to pH 2 with concentrated hydrochloric acid, then extracted into methylene chloride (3×20 mL). The combined organic extracts were dried over Na₂SO₄, and then concentrated in vacuo to provide the carboxylic acid as waxy oil, which was purified by flash chromatography (95:5 CH₂Cl₂/MeOH) to give the desired product which was solidified from pet. ether to give white solid (10j) 0.15 g in 75% yield, mp: 58.8° C.? ¹H NMR (CDCl₃) δ 7.35 (d, 2H), 7.27 (m, 2H), 7.08 (d, 2H), 6.88 (d, 2H), 6.82 (d, 2H), 6.78 (d, 2H), 4.91 (s, 2H), 4.61 (s, 2H), 3.77 (s, 3H), 2.54 (t, 2H), 2.33 (s, 3H), 1.85 (t, 2H), 1.55 (s, 6H), 1.29 (s, 9H); ¹³C NMR (CDCl₃) δ 172.0, 156.3, 151.2, 149.8, 147.7, 133.7, 132.6, 130.6, 126.3, 125.8, 125.1, 125.0, 122.6, 117.6, 111.4, 77.1, 52.4, 45.9, 41.4, 31.6, 28.4, 24.3, 22.4, 22.2. Anal. Calcd for C₃₄H₄₁N₃O₅.½H₂O: C, 70.32; H, 7.12; N, 7.23. Found: C, 70.51; H, 7.26; N, 7.17.

### 2-(4-{3-[4-(4-Methoxy-benzyl)-1-(4-trifluoromethyl-benzyl)-5-oxo-4,5-dihydro-1H[1,2,4]triazol-3-yl]-propyl}-phenoxy)-2-methyl-propionic acid (10k)

A solution of 2-(4-{3-[4-(4-Methoxy-benzyl)-1-(4-trifluoromethyl-benzyl)-5-oxo-4,5-dihydro-1H[1,2,4]triazol-3-yl]-propyl}-phenoxy)-2-methyl-propionic acid methyl ester (9k) (0.72 g, 0.0012 mol) in methanol (7.5 mL) was treated with 2N NaOH (5 ml) and the mixture stirred overnight at ambient temperature. After concentration to dryness, the residue was dissolved in methylene chloride (25 ml), water (10 mL), the solution acidified to pH 2 with concentrated hydrochloric acid, then extracted into methylene chloride (3×25 mL). The combined organic extracts were dried over Na₂SO₄, and then concentrated in vacuo to provide the carboxylic acid as waxy oil, which was purified by flash chromatography (95:5 CH₂Cl₂/MeOH) to give the desired product which was solidified from pet. ether to give white solid (10k) 0.62 g in 89% yield, mp: 45.9° C. ¹H NMR (CDCl₃) δ 7.59 (d, 2H), 7.45 (d, 2H), 7.07 (d, 2H), 6.86 (d, 2H), 6.82 (d, 2H), 6.75 (d, 2H), 4.98 (s, 2H), 4.61 (s, 2H), 3.78 (s, 3H), 2.54 (t, 2H), 2.35 (s, 3H), 1.87 (t, 2H), 1.51 (s, 6H); ¹³C NMR (CDCl₃) δ 174.2, 156.4, 151.2, 149.9, 143.8, 137.5, 132.4, 127.2, 127.0, 126.2, 125.8, 125.5, 124.9, 122.8, 117.5, 111.4, 77.2, 52.4, 45.6, 41.4, 31.1, 24.4, 22.3, 22.2. Anal. Calcd for C₃₁H₃₂F₃N₃O₅.½H₂O: C, 62.82; H, 5.44; N, 7.09. Found: C, 62.60; H, 5.34; N, 6.94.

### 2-(4-{3-[4-(4-Methoxy-benzyl)-1-(4-trifluoromethox-benzyl)-5-oxo-4,5-dihydro-1H[1,2,4]triazol-3-yl]-propyl}-phenoxy)-2-methyl-propionic acid (101)

A solution of 2-(4-{3-[4-(4-Methoxy-benzyl)-1-(4-trifluoromethoxy-benzyl)-5-oxo-4, 5-dihydro-1H[1,2,4]triazol-3-yl]-propyl}-phenoxy)-2-methyl-propionic acid methyl ester (91) (0.67 g, 0.0011 mol) in methanol (7.5 mL) was treated with 2N NaOH (6 ml) and the mixture stirred overnight at ambient temperature. After concentration to dryness, the residue was dissolved in methylene chloride (25 ml), water (10 mL), the solution acidified to pH 2 with concentrated hydrochloric acid, then extracted into methylene chloride (3×20 mL). The combined organic extracts were dried over Na₂SO₄, and then concentrated in vacuo to provide the carboxylic acid as waxy oil, which was purified by flash chromatography (95:5 CH₂Cl₂/MeOH) to give the desired product which was solidified from pet. ether to give white solid (101) 0.60 g in 91% yield, mp: 58.8° C. ¹H NMR (CDCl₃) δ 7.34 (d, 2H), 7.13 (d, 2H), 7.06 (d, 2H), 6.81 (d, 2H), 6.77 (d, 2H), 6.69 (d, 2H), 4.88 (s, 2H), 4.60 (s, 2H), 3.71 (s, 3H), 2.46 (t, 2H), 2.32 (s, 3H), 1.80 (t, 2H), 1.31 (s, 6H); ¹³C NMR (CDCl₃) δ 177.5, 156.4, 151.1, 149.9, 145.8, 143.6, 132.6, 132.4, 126.6, 125.9, 125.7, 124.9, 119.1, 118.5, 118.2, 111.4, 78.9, 52.3, 45.3, 41.4, 31.2, 24.3, 22.5, 22.3. Anal. Calcd for C₃₁H₃₂F₃N₃O₆.3/2H₂O: C, 59.41; H, 5.14; N, 6.70. Found: C, 59.30; H, 5.13; N, 6.57.

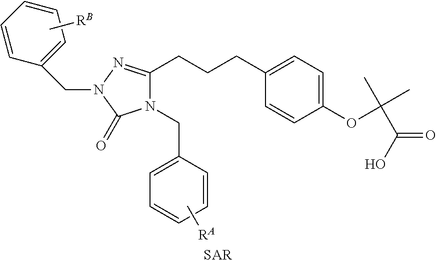

Synthesis of a focused small chemical library. A focused small chemical diverse library would provide structure-activity relationships (SAR) designed for the PPARα selective agonism modulators. To increase the likelihood of “druggability”, specific R^{A} and R^{B} groups were selected such that the final compounds adhere to Lipinski's rules (molecular weights <500 Da, calculated octanol/water partition coefficients (ClogP)<5 and less than 5 H-bond donors and 10 H-bond acceptors). Additional selection criteria included chemical compatibility, structure diversity, inclusion of drug-like substructures and commercial availability of starting materials. We propose to incorporate different R^{A} and R^{B} groups into several positions that fill the critical cavity of PPARα. The chemical structures of library was characterized by ¹H and ¹³C NMR and element analysis and their activity data were used to build SAR profiles.

### Example 2. Biological Evaluations

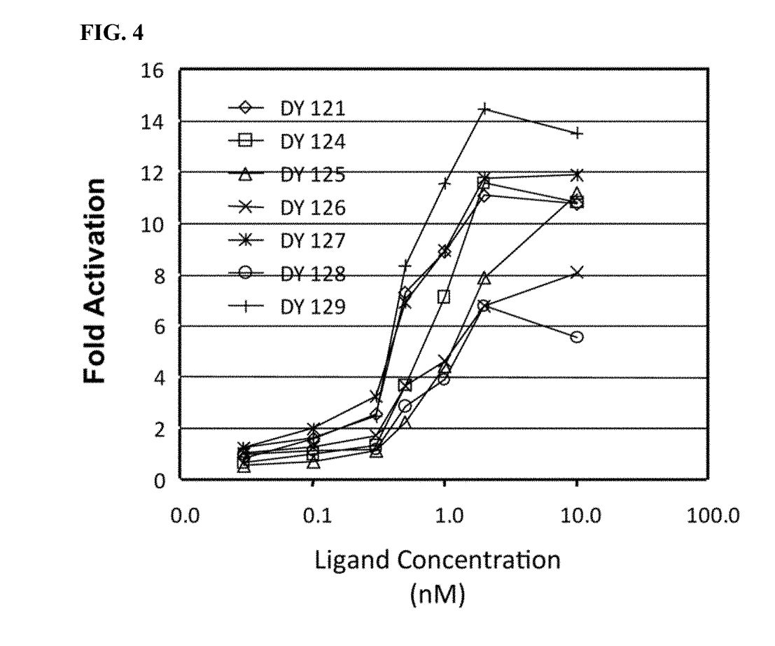

To assess the functional transcriptional activity of these compounds, cell-based assays using chimeric receptor Gal4 DNA-binding domain (DBD)-NR ligand binding domain cotransfection assay (LBDs of PPARα, PPARγ, and PPARδ) were performed. As an initial screen to determine the PPARα activity and selectivity, all compounds were evaluated for in vitro potency and selectivity by transfection testing, using CV-1 cells transfected with human and mouse PPARs. MH100×4 is a luciferase reporter with four copies of a GAL4 UASG response element, where GAL-I refers to the ligand-binding domain of the indicated receptor fused to the C-terminus of the GAL4 DNA-binding domain. Assays were performed using a Biomek automated workstation in which the genes for the nuclear receptor, as well as a plasmid containing a response element upstream of a luciferase cDNA, are transfected into CV-1 cells. Compounds were evaluated for their ability to activate human and mouse PPARs at 0.03, 0.1, 0.5, 1, 2.5, 3, and 10 M test concentrations. Those compounds that showed good PPARα potency and selectivity were evaluated at additional concentrations. DY121 has been identified as the most potent and high affinity PPARα modulator and used as a known PPARα agonist to compare the activity of unliganded PPARs. A series of synthetic compounds of this focused library has been identified and showed similar activities at the 1 M concentration as compared with DY121. DY121 was also identified as selective for hPPARα over the other subtypes from a functional perspective (EC₅₀) and is demonstrated sufficient potency and selectivity for the hPPARR receptor over other hPPAR isoforms to warrant further investigation in vivo. Mammalian transient transfections demonstrated activation of a reporter on a PPAR response element (FIG. 4). Differences in fold activation suggest selective modulation of the PPARα receptor. The transactivation activity of the series of compounds (DY121-132, 10a-g) toward PPARα was expressed as EC₅₀, which is the concentration of the test compound that affords half-maximum activity, as summarized in Table 1. All compounds listed in Table 1 (10a-g) were inactive at up to 10-30 uM with regard to PPARγ and PPARδ.

Cell Culture.

Cell-based reporter gene assays Cell based transactivation assays were performed in CV 1 cells as described (Forman, et al.). PPARα activity was assayed with a GAL4 reporter construct and fusion proteins containing the ligand binding domains of human PPARα, human PPARγ and human PPARδ linked to the DNA binding domain of yeast GAL4. Reporter constructs (300 ng/10⁵ cells) and cytomegalovirus-driven expression vectors (20-50 ng/10⁵ cells) were added as indicated along with CMX-b-gal (500 ng/10⁵ cells) as an internal control. Cells were transiently transfected by Lipofectamine as described. Cells were incubated with DNA complexed liposomes for 2 h and subsequently treated for approximately 45 h with phenol red free DMEM FBS containing the indicated compounds. After exposure to ligand, the cells were harvested and assayed for luciferase and β-galactosidase activity. All points were assayed in triplicate and varied by less than 15%. Each experiment was repeated three or more times with similar results. Fold activation is reported.

In vitro binding and transfection studies. As an initial screen to determine the PPARα, PPARγ, and PPARδ activity, all compounds were evaluated for in vitro potency and selectivity by transfection testing, using CV-1 cells transfected with human and mouse PPARs. MH100×4 is a luciferase reporter with four copies of a GAL4 USAG response element, where GAL-I refers to the ligand-binding domain of the indicated receptor fused to the C-terminus of the GAL4 DNA-binding domain. Assays were performed using a Biomek automated workstation in which the gene for the nuclear receptor, as well as a plasmid containing a response element upstream of a luciferase cDNA, are transfected into CV-1 cells. Compounds were evaluated for their ability to activate human PPARs at 1, 2.5, 3, and 5 uM test concentrations. Those compounds that showed good PPARα actication were evaluated at additional concentrations.

Plasmid Construction.

Expression plasmids containing the GAL4 DNA-binding domain (GAL4) fused to the PPARα-ligand-binding domain (LBD) (159-468 a.a.), PPARγ/δ-LBD (136-441 a.a.), and PPARγ-LBD (157-475 a.a.), as well as the PPRE-driven luciferase reporter plasmid (pPPRE-Luc), were constructed as previously reported.²⁰

Quantitative Real-Time PCR.

The following oligonucleotide primers were used to amplify insertion sequences: for PPARα, FW-5′-GCGGGATCCGTGGACACGGAAAGCCCACTCTGCCCC-3′ (SEQ ID NO:1) and Rev-5′-ATCTCAGTACATGTCCCTGTAGATCTCCTGCA-3′ (SEQ ID NO:2); for RXRα, FW-5′ ATCATGGACACCAAACATTTCC-3′ (SEQ ID NO:3) and Rev-5′-CTAAGTCATTTGGTGCGG-3′ (SEQ ID NO:4). It has been well established that PPARα agonists fibrates reduce plasma triglycerides largely by inducing hepatic PPARα-mediated transcription of key proteins in hepatic long-chain fatty acid 3-oxidation, such as CPT1 (carnitine palmitoyltransferase-1). DY121 promoted the expression of PPARα, CPT1, and LPL involved in free fatty acid β-oxidation. The mRNA expressions of the PPARα-regulated genes CPT-Ia and ACADM were measured by real-time quantitative-PCR (RT-qPCR). These data indicate that DY121 might be a gene-selective PPARα modulator, which is distinct from that of the well-known PPARα agonist WY14643. A structural specificity of DY121 may be responsible for this regulation. PPARα is predominantly expressed in liver. PPARα modulates fatty acid catabolism in the liver. We next measured the mRNA expression of PPARα target genes in the liver. PPARα agonists exert their actions and attributed to the activation of PPARα which in turn effects an increase in LPL (lipoprotein lipase) gene expression and transrepression of apoC-III (apolipoprotein C-III). Activation of PPARα by its modulators can increase PPARα levels and activate PPARα pathways, leading to increased fatty-acid oxidation and reduction of oxidative stress. DY121 and its analogs induced the expression of PPARα-regulated genes involved in lipid, glucose, and cholesterol metabolism, such as ACOX1 (acyl-CoA oxidase) and Cyp4A3 (cytochrome P450 gene subfamily). Members of the CYP4A gene subfamily catalyze the oxygenation of biologically important fatty acids. CYP4A enzymes, which are expressed in the liver, kidney and heart, Interestingly, treatment with DY121 and its analogs result in enhanced levels of CYP4A3 mRNA in the liver of wild-type but not PPARα knockout mice, indicating that in mouse the expression of CYP4A3 genes is under the control of PPARα. These results show that DY121 induces the expression of PPARα target genes in the liver.

Oil Red O Staining.

Cells on day 7 were washed twice with PBS, fixed with 10% neutral buffered formalin for 1 h at room temperature, washed with PBS and then dried completely. The fixed cells were stained with Oil Red O in an isopropanol/distilled water (6:4) solution for 30 min at room temperature and then washed twice with distilled water. The stained lipid droplets were observed, and microscopic images were obtained from randomly selected fields under a phase contrast microscope (AX70; Olympus, Tokyo, Japan) equipped with a digital camera and processed using ImagePro Plus software (Media Cybernetics, Silver Spring, Md., USA).

Statistical Analysis.

All quantitative data are presented as mean±S.E.M or mean±S. D. Significant differences between mean values were determined. A p-value of ≤0.05 was considered significant and is denoted by asterisks (*).

### Example 3. Structure-Activity Relationships (SAR)

The SAR results are summarized in Table 1. The introduction of a bulky benzyl substituent at the N⁴-position of the triazolone core was effective to improve PPARα agonistic activity and selectivity, as expected; having a benzyl group connected to the triazolone at the N⁴ position is a prerequisite for exhibiting high binding affinity and potency on the PPARα. Interestingly, while the introduction of a substituent at the N⁴-position is important to elicit PPARα agonistic activity (DY121 vs LY), the activity seems to correlate well with the benzyl bulkiness of the substituent.

The modification that significantly affects the selectivity and activity for the PPARα receptor is important at the position N⁴ to the triazolone core. It can be seen that a benzyl substituted (of the triazolone) at the N⁴ position increase PPARα agonistic activity and selectivity. In our experiments variation of a side chain on the benzyl substituted at the N⁴ position appeared to be beneficial for PPARα. All of the resulting compounds having benzyl substituted at the N⁴ position in triazolone were found to possess high potency on the PPARα. The data are shown in Table 1 with a select set only. This study confirmed that the introduction of benzyl substituted at the N⁴ position in triazolone requires to providing generally improved potencies on the PPARα.

Since compound DY121 (10a) had exhibited potent and subtype selective human PPARα activation, we selected it as a model compound and made analogs with different substituents on the para, ortho, and meta positions of the phenyl ring of benzyl substituted at the N⁴ position (Scheme 1, 2, and 3). As shown in Table 1, introduction of a 4-methoxy (10a), 4-methyl (10e), and 2-chloro (10 g) substituents on benzyl moiety of triazolone enhanced PPARα transactivation activity, and afforded activity greater than that of 10b, 10c, 10d and 10f. The bulkier substituent on 10f decreased the activity slightly. When there was a 3-methoxy constituent in the ortho position (10b) the activity was slightly less than that of its para counterpart. The potency decreased in the order of 4-MeO (10a)≥4-Me (10e)=2-Cl (10 g)≥H (10c)=4-F (10d)≥3-MeO (10b)≥3,4,5-MeO (10f). These data suggest that there are distinct steric requirements to be met for potent human PPARα transactivation activity. The effect of these analogs with benzyl substitution at N⁴ on PPARα activity was active at a concentration as low as 0.0003 uM. The study shows that benzyl substitution on the N⁴ position of the triazolone core is important for potent PPARα transactivation potency. The enhancement of the agonistic effect of compounds 10a-g in replacing the hydrogen (LY518674) on the N⁴ with benzyl substitution could be due to the hydrophobicity of the benzyl substituents. These substituents, because of conformational flexibility, would be able to establish more interactions with the receptor, suggested that interactions with the aromatic ring R⁴ region plays an important roles in determining PPARα functional activity and selectivity in this portion of the molecule: Therefore, the beneficial effect of benzyl substitution on the N⁴ position of the triazolone core is confirmed.

SAR studies revealed that an aromatic moiety, an electron-donating substituent (DY121, OCH₃), and a super conjugated group (DY127, CH₃) on the para-position of the benzyl ring at the N⁴ position are critical for high PPARα affinity. Further addition of a halogen atom at the ortho position of the benzyl moiety substitution (10 g, DY129) was also found to be important for a potent agonist of PPARα and effective in improving PPARα agonistic activity. This SAR study of the “electron donor” benzyl ring substituents showed that the 4-OCH3-benztl moiety was optimal, providing potent PPARα activity and the highest level of PPARα selectivity. Thus, we have successfully obtained a potent, selective, structurally novel PPARα agonist, DY121. Compound DY121 and its analogues selectively agonize PPARα with EC₅₀ of 1 nM. As compared to the LY518674, DY121 showed 42-fold greater PPARα agonistic activity,

TABLE 1
A select set of DY121 analogs on human and mouse PPARα
transactivation potency.

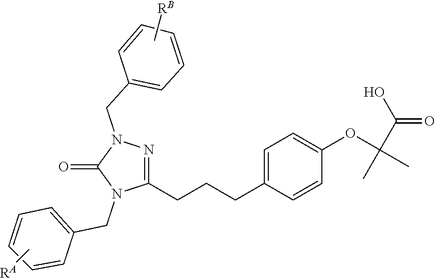

| | | | EC₅₀(nM) | |
|---|---|---|---|---|
| comp | R^{A} | R^{B} | hPPARα | mPPARα |
| 10a | 4-MeO | 4-Me | 0.85 | 3 |
| DY121 | | | | |
| 10b | 3-MeO | 4-Me | 2 | 20 |
| DY124 | | | | |
| 10c | H | 4-Me | 1.5 | 20 |
| DY125 | | | | |
| 10d | 4-F | 4-Me | 1.5 | 10 |
| DY126 | | | | |
| 10e | 4-Me | 4-Me | 1 | 3 |
| DY127 | | | | |
| 10f | 3,4,5-Tri- | | | |
| DY128 | MeO | 4-Me | 3 | 30 |
| 10g | 2-Cl | 4-Me | 1 | 10 |
| DY129 | | | | |
| 10h | 4-vinyl | 4-Me | 12 | 20 |
| DY130 | | | | |

Compounds were screened for agonist activity towards PPAR-GAL4 chimeric receptors in transiently transfected CV-1 cells as described. EC₅₀ is the concentration of test compound that gave 50% maximum efficacy.

In brief, monkey kidney CV-1 cells were co-transfected with appropriate reporter constructs and expression vectors. The fold activation of the reporter construct by ligand was determined at several concentrations of selected compounds (FIG. 4).²⁰ To examine the effect of DY-compound on PPARα-mediated PPRE transcriptional activation, a PPRE-luc reporter assay was conducted using a PPRE-driven luciferase reporter-gene (PPRE-luc) plasmid and PPARα and RXRα gene expression plasmids in CV-1 cells. DY-compound significantly increased PPRE-luc activity in cells that were co-transfected with the PPARα and RXRα gene expression plasmids, indicating that DY-compound not only activated PPARα but also promoted PPARα-mediated PPRE transcriptional activation. As shown by the luciferase reporter assays, the highest PPARα activation was observed with DY121, DY127, and DY129.

### Example 4. In Vivo Studies

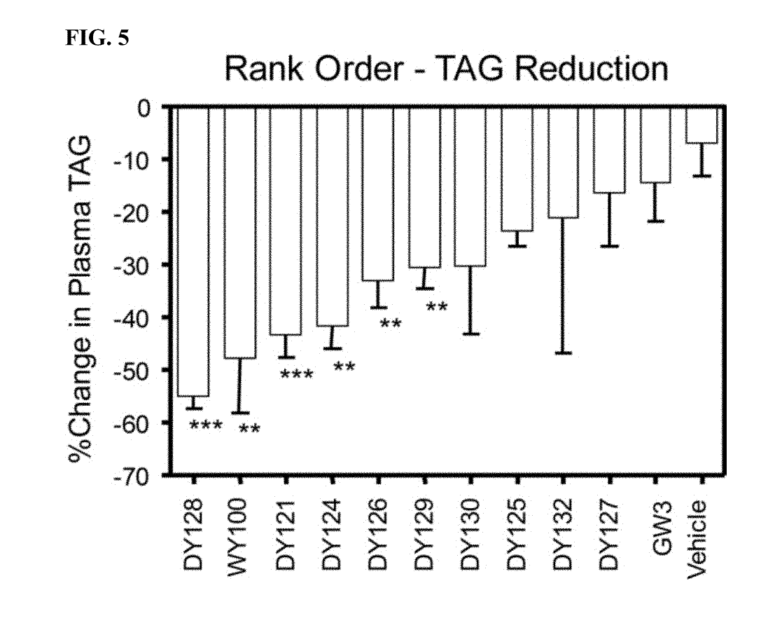

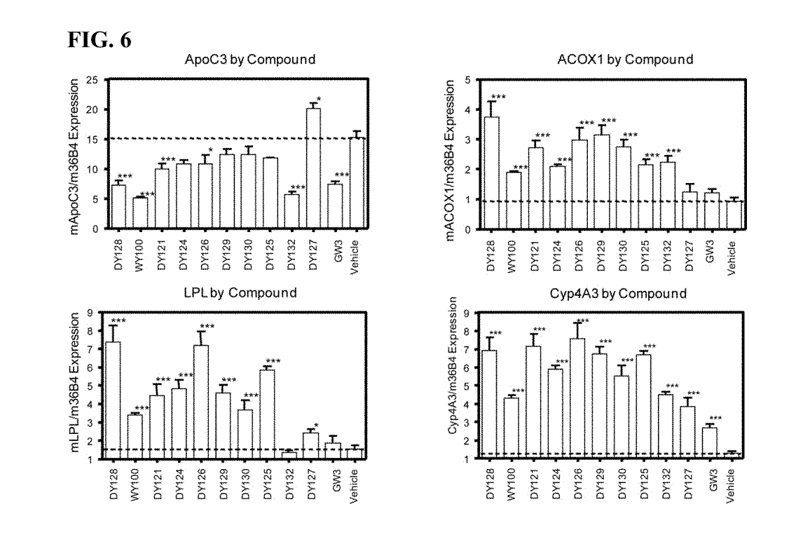

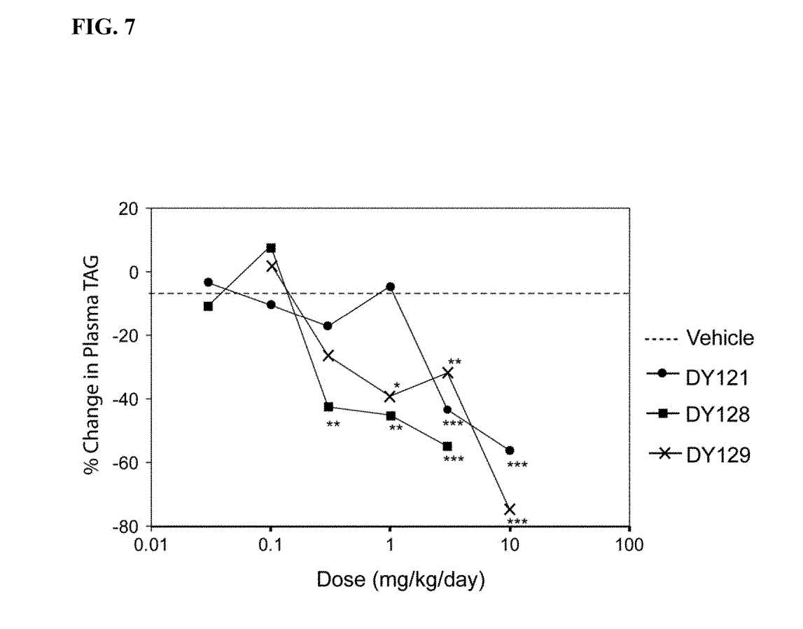

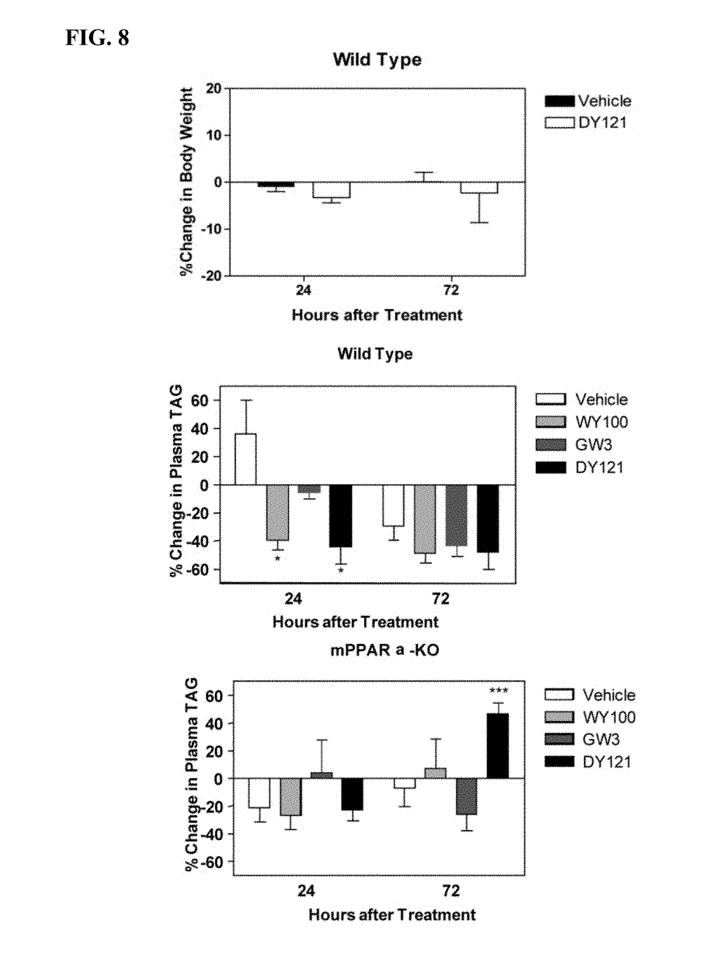

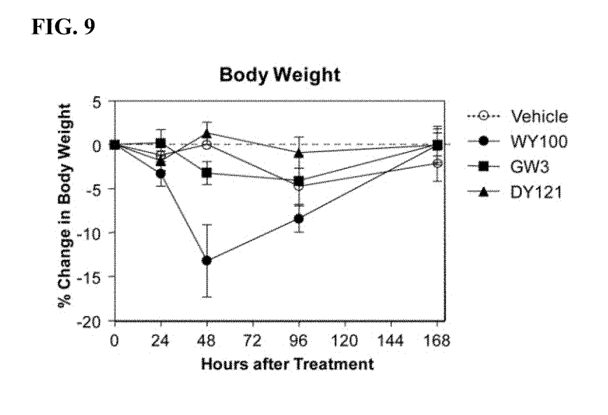

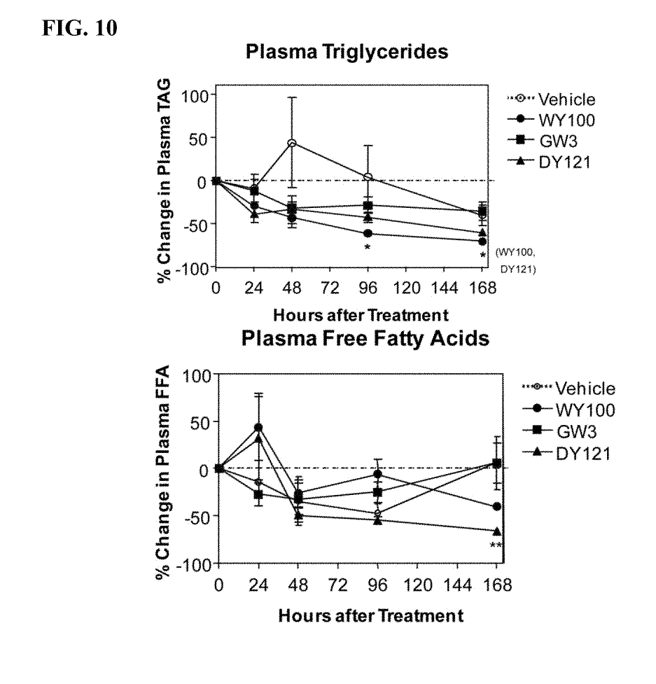

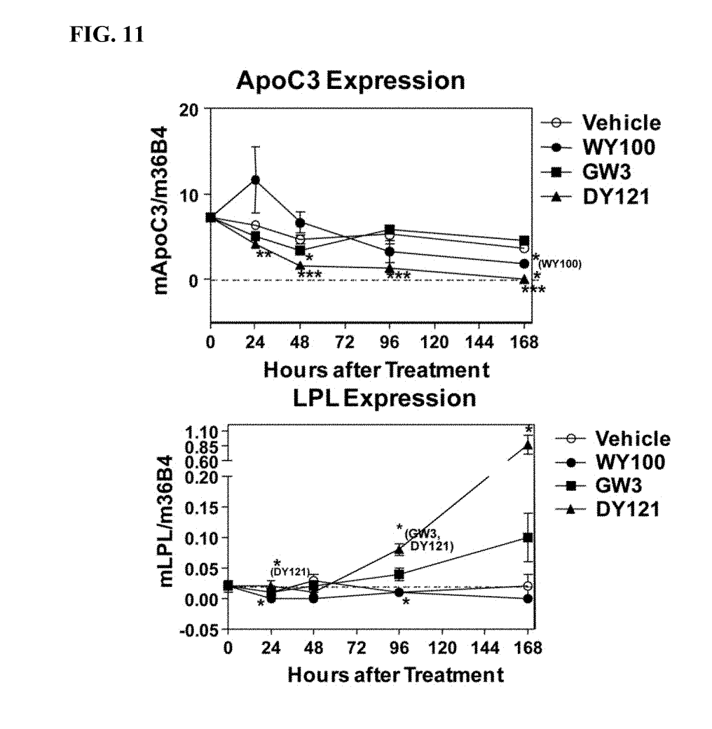

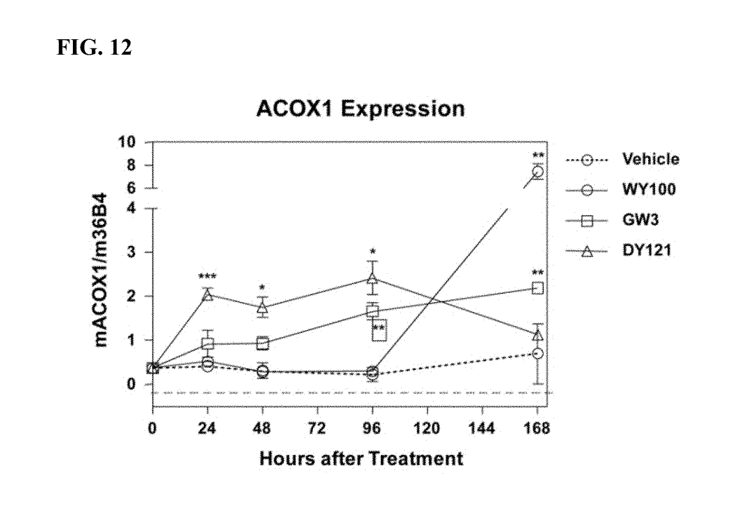

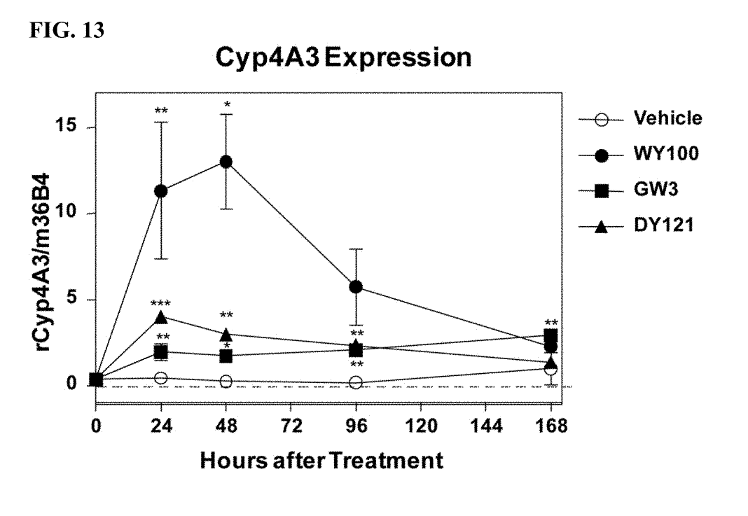

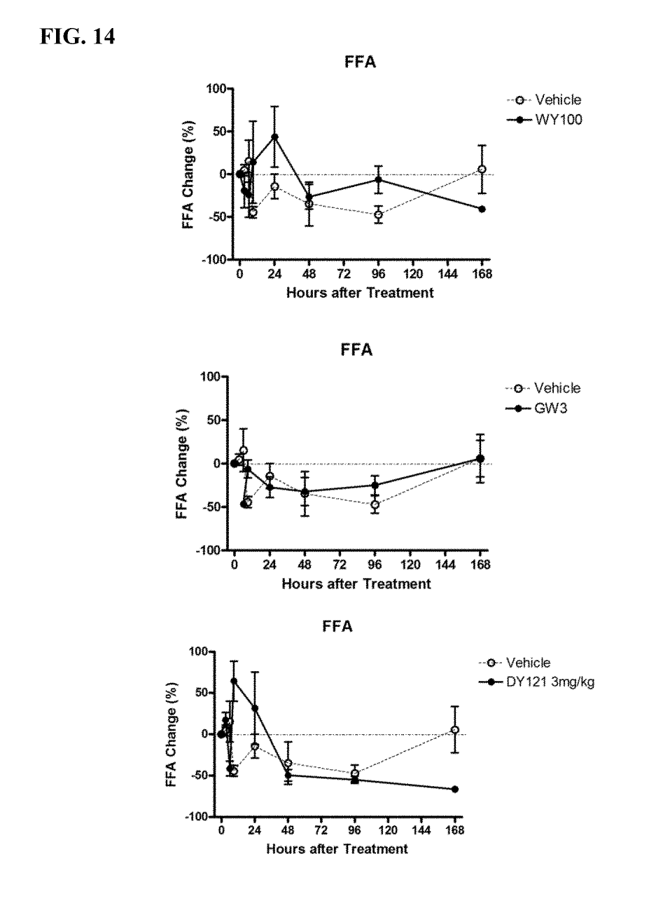

In this study, we identified DY121 and its analogs that possessed potent and selective PPARα agonist activity. Next, we would like to investigate the therapeutic potential of DY121 to see if it could be candidate for treating NAFLD/NASH. Note that the pathogenesis of NASH has not yet been fully elucidated so far; however, the ‘two-hit’ theory is widely accepted. The first hit is the accumulation of fatty acids in the liver to cause steatosis. We selected DY121 and its analogs as potent PPARα modulators for subsequent animal experiments to investigate the anti-dyslipidemic effects and found that they exerted a recognizable anti-dyslipidemic effect in vivo. The newly produced PPARα agonist DY121 and its analogs, as well as WY100=WY14643 and GW3 (GW7647), were evaluated in C₅₇Bl/6J female mice for their ability to alter serum triglyceride, liver gene expression, and glucose. Fibric acid derivatives (100 mg/kg/day) was used as a control, WY100 (WY14, 643, 100 mg/kg/day), GW3 (GW7647-positive control, 3 mg/kg/day), DY121 (˜3 mg/kg/day), respectively. DY series compounds and controls administered once to C₅₇Bl/6J female mice at 8 weeks of age. Mice were treated at 3 mg/kg for all compounds except WY14643 (100 mg/kg). DY121 and other-treated groups are shown in FIG. 5-14.

Administration of DY121 improves dyslipidemia and hepatic steatosis (NAFLD/NASH) by regulating the expression of genes involved in hepatic lipid metabolism in C₅₇Bl/6J female mice. The body weights and food intake showed no significant differences between the control and the DY121-containing groups. However, serum triglyceride (TG) concentrations significantly decreased in the DY121-treated mice. Also, TG accumulation was suppressed in the DY121-treated mice. The suppression was more apparent in the mice treated with 3 mg/kg DY121 than WY14643 (100 mg/kg). These results indicate that DY121 treatment suppresses hyperlipidemia and hepatic TG accumulation. Dyslipidemia is a direct risk factor for NAFLD/NASH disease and is characterized by a high level of triglycerides (TGs) and a low level of high-density lipoprotein (HDL) cholesterol. PPARα is expressed primarily in the liver and skeletal muscle and promotes fatty acid β-oxidation to reduce the amount of TGs in the blood, liver, and skeletal muscle. By acting as PPARα modulators, DY121 and its analogs could be important candidates for potential anti-hyperlipidemic drugs down-regulate plasma TGs and up-regulate HDL-cholesterol. DY121 and its analogs show dose-dependent anti-dyslipidemic effects in vivo and show the results of the experiments that were conducted to confirm the reproducibility and dose dependence of the effects of suppression of triglycerides.

### Example 5. Computational Modeling of PPARs and Binding of Compound DY121 (10a) to PPARα

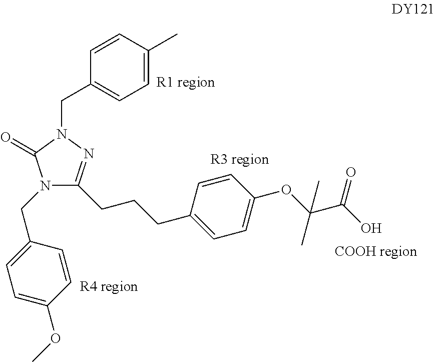 [SMILES: COc1ccc(Cn2c(CCCc3ccc(OC(C)(C)C(=O)O)cc3)nn(Cc3ccc(C)cc3)c2=O)cc1]

The labels R1, R1 region, R3, R3 region, R4, and R4 region refer to the labeled portions of the compound disclosed herein, as shown immediately above using DY121 as an example. The labels R1, R1 region, R3, R3 region, R4, and R4 region are not interchangeable with the variables R1, R3, and/or R4 used to describe substituents of the compounds described herein, including in aspects and embodiments. The labels R1, R1 region, R3, R3 region, R4, and R4 region are used to refer to portions of the compounds that form particular interactions when contacting a protein.

Furthermore, we performed in silico calculations to understand the possible binding poses and ligand-protein interactions of the inhibitors on PPAR proteins. By implementing induced fit docking methodology from Schrödinger software, we docked compound DY121 and LY518674 to PPARα, PPARγ and PPARγ proteins to evaluate the affinity and selectivity of the inhibitors to PPARα. Induced fit docking method allows the side-chain of residues to be flexible to gain better interaction with ligand. Thus it can reproduce the most important ligand-interaction regions (R1 region/R4 region/carboxyl group (COOH region)) for PPARα inhibitor in X-ray structure as depicted in FIG. 19.

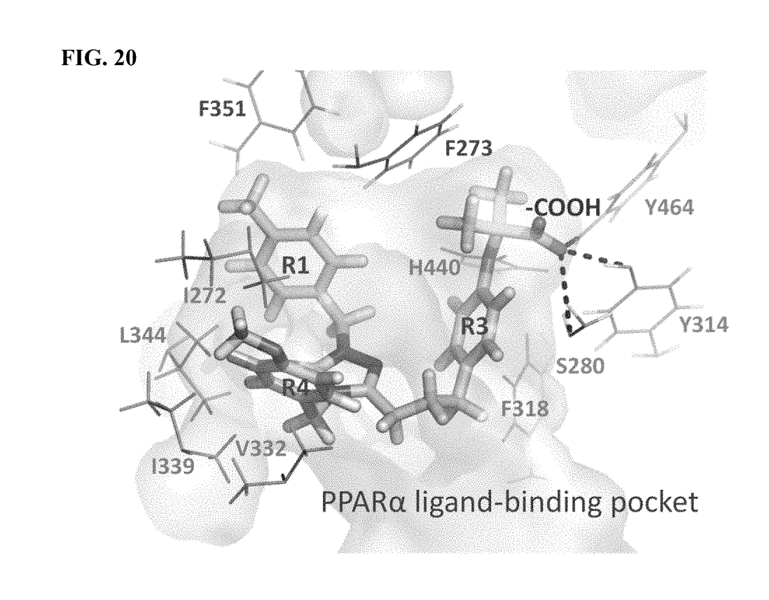

As shown in FIG. 20, the carboxyl group of DY121 can form hydrogen bonds with S280, Y314 and Y464 in PPARα protein. The aromatic ring R3 region forms strong 7t-interaction network with F318 and H440. The aromatic ring R1 region forms π-stacking interaction with F273 and F351. The unique arm of R4-region of DY121 forms extra hydrophobic interaction with I272, V332, I339, and L344, which can contribute more binding affinity to make DY121 a more efficacious PPARα agonist. For example, the docking score of DY121 is as large as −15.4 kcal/mol, which demonstrates much higher binding affinity than that of LY518674 with a lower docking score of −13.6 kcal/mol on PPARα.

PPAR has a large ligand binding pocket with a volume of ˜1,011 Å³. Compound DY121 fits well within the pocket and forms more extensive π-stacking and hydrophobic interactions with PPARα in ligand binding domain which results in higher binding affinity over PPARγ (docking score of −14.5 kcal/mol) and PPARγ (−14.9 kcal/mol). These results suggest that DY121 is a subtype-selective PPARα agonist.

### REFERENCES

1. Forman, B. M., Chen, J., Evans, R. M., Hypolipidemic drugs, polyunsaturated fatty acids, and eicosanoids are ligands for peroxisome proliferator-activated receptors alpha and delta. Proc. Natl. Acad. Sci. U.S.A., 1997, 94, 4312-4317.
2. Willson, T.; Beown, P.; Sternbach, D.; Henke, B. The PPARs: From Orphan Receptors to Drug Discovery. J. Med. Chem. 2000, 43, 527-550.
3. Francque, S. et al., PPAR gene expression correlates with severity and histological treatment response in patients with non-alcoholic steatohepatitis, Journal of Hepatology 2015, 63, 164-173.
4. Pawlak, M.; Lefebvre, P.; Staels, B. Molecular mechanism of PPAR action and its impact on lipid metabolism, inflammation and fibrosis in non-alcoholic fatty liver disease. J. Hepatol. 2015, 62, 720-733.
5. Kersten, S.; Stienstra, R. The role and regulation of the peroxisome proliferator activated receptor alpha in human liver, Biochimie, 2017, 136, 75-84.
6. Balas B, Belfort R, Harrison S A, et al. Pioglitazone treatment increases whole body fat but not total body water in patients with non-alcoholic steatohepatitis. J Hepatol. 2007, 47, 565-570.
7. Lefebvre, P., Chinetti, G., Fruchart, J.-C. & Staels, B. Sorting out the roles of PPAR in energy metabolism and vascular homeostasis. J. Clin. Invest. 2006, 116, 571-580.
8. Abdelmegeed, M. A. et al. PPARα expression protects male mice from high fat-induced nonalcoholic fatty liver. J. Nutr. 2011, 141, 603-610.
9. Staels, B.; Dallongeville, J.; Auwerx, J.; Schoonjans, K.; Leitersdorf, E.; Fruchart, J. C. Mechanism of Action of Fibrates on Lipid and Lipoprotein Metabolism. Circulation 1998, 98, 2088-2093.
10. Fruchart, J.-C. Selective peroxisome proliferator-activated receptorα modulators (SPPARMα): The next generation of peroxisome proliferator-activated receptor α-agonists. Cardiovasc. Diabetol. 2013, 12, 82.
11. Ishibashi, S. et al. Effects of K-877, a novel selective PPARα modulator (SPPARMα), in dyslipidaemic patients: a randomized, double blind, active- and placebo-controlled, phase 2 trial. Atherosclerosis 2016, 249, 36-43.
12. Huss, et al., Biochimica et Biophysica Acta, 2015, 1912-1927.
13. Stienstra, R. et al. Peroxisome proliferator-activated receptor α protects against obesity-induced hepatic inflammation. Endocrinology 2007, 148, 2753-2763.
14. Roberts L. D, Murray A J, Menassa D, Ashmore T, Nicholls A W, Griffin J L: The contrasting roles of PPARdelta and PPARgamma in regulating the metabolic switch between oxidation and storage of fats in white adipose tissue. Genome Biol 2011, 12, R75.
15. Abdelmegeed, M. A. et al. PPARα expression protects male mice from high fat-induced nonalcoholic fatty liver. J. Nutr. 2011, 141, 603-610.
16. Fujita, K. et al. Dysfunctional very-low-density lipoprotein synthesis and release is a key factor in nonalcoholic steatohepatitis pathogenesis. Hepatology 2009, 50, 772-780.
17. Brown, P. J.; Winegar, D. A.; Plunket, K. D.; Moore, L. B.; Lewis, M. C.; Wilson, J. G.; Sundseth, S. S.; Koble, C. S.; Wu, Z.; Chapman, J. M.; Lehmann, J. L.; Kliewer, S. A.; Willson, T. M. An ureido-thioisobutyric acid (GW 9578) is a subtype-selective PPARα agonist with potent lipid-lowering activity. J. Med. Chem. 1999, 42, 3785-3788.
18. Normura, M.; Tanase, T.; Ide, T.; Tsunoda, M.; Suzuki, M.; Uchiki, H.; Murakami, K.; Miyachi, H. Design, synthesis, and Evaluation of Substituted Phenylpropanoic Acid Derivatives as Human Peroxisome Proliferator Activated Receptor Activators. Discovery of Potent and Human Peroxisome proliferator Activated Receptor a Subtype-Selective Activators. J. Med. Chem. 2003, 46, 3581-3599.
19. Xu, Y.; Mayhugh, D.; Saeed, A.; Wang, X.; Thompson, R. C.; Dominianni, S. J.; Kauffman, R. F.; Singh, J.; Bean, J. S.; Bensch, W. R.; Barr, R. J.; Osborne, J.; Montrose-Rafizadeh, C.; Zink, R. W.; Yumibe, N. P.; Huang, N.; Luffer-Atlas, D.; Rungta, D.; Maise, D. E.; Mantlo, N. B. Design and Synthesis of a Potent and Selective Triazolone-Based Peroxisome Proliferator-Activated Receptor Agonist. J. Med. Chem. 2003, 46, 5121-5124.
20. Barry Forman, et al., Endogenous Bile Acids Are Ligands for the Nuclear Receptor FXR/BAR, Mol Cell. 1999, 3, 543-553.
21. Patrick Markt, et al., Discovery of Novel PPAR Ligands by a Virtual Screening Approach Based on Pharmacophore Modeling, 3D Shape, and Electrostatic Similarity Screening, J. Med. Chem. 2008, 51, 6303-6317.
22. Sherman, W.; Day, T.; Jacobson, M. P.; Friesner, R. A.; Farid, R., “Novel Procedure for Modeling Ligand/Receptor Induced Fit Effects,” J. Med. Chem., 2006, 49, 534-553.
23 Xu, H. E., Lambert, M. H., Montana, V. G., Plunket, K. D., Moore, L. B., Collins, J. L., Oplinger, J. A., Kliewer, S. A., Gampe Jr., R. T., McKee, D. D., Moore, J. T., Willson, T. M. “Structural determinants of ligand binding selectivity between the peroxisome proliferator-activated receptors.” (2001) Proc. Natl. Acad. Sci. USA 98: 13919-13924

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having the formula: 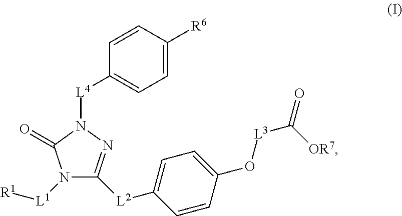
wherein,
L¹ is a bond, —C(O)—, —C(O)O—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is unsubstituted C₁-C₆ alkylene;
L³ is substituted or unsubstituted C₁-C₆ alkylene;
L⁴ is unsubstituted C₁-C₄ alkylene;
R¹ is substituted or unsubstituted cycloalkyl, substituted or unsubstituted heterocycloalkyl, substituted or unsubstituted aryl, or substituted or unsubstituted heteroaryl;
R⁶ is unsubstituted C₁-C₄ alkyl; and
R⁷ is hydrogen or, together with the oxygen to which it is attached, forms a prodrug moiety.

2. The compound of claim 1, having the formula:

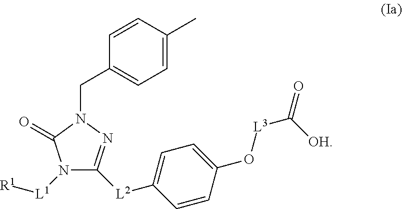

3. The compound of one of claims 1 to 2, wherein L³ is substituted or unsubstituted C₁-C₃ alkylene.

4. The compound of one of claims 1 to 2, wherein L³ is substituted or unsubstituted C₁-C₃ alkylene.

5. The compound of one of claims 1 to 2, wherein L³ is —C(CH₃)₂—.

6. The compound of one of claims 1 to 5, wherein L² is unsubstituted C₂-C₄ alkylene.

7. The compound of one of claims 1 to 5, wherein L² is unsubstituted n-propylene.

8. The compound of one of claims 1 to 7, wherein L¹ is a bond or substituted or unsubstituted alkylene.

9. The compound of one of claims 1 to 7, wherein L¹ is a bond or unsubstituted C₁-C₃ alkylene.

10. The compound of one of claims 1 to 7, wherein L¹ is an unsubstituted C₁-C₃ alkylene.

11. The compound of one of claims 1 to 7, wherein L¹ is an unsubstituted methylene.

12. The compound of one of claims 1 to 11, wherein R¹ is substituted or unsubstituted C₄-C₆ cycloalkyl, substituted or unsubstituted 4 to 6 membered heterocycloalkyl, substituted or unsubstituted phenyl, or substituted or unsubstituted 5 to 6 membered heteroaryl.

13. The compound of one of claims 1 to 11, wherein R¹ is substituted or unsubstituted phenyl or substituted or unsubstituted 5 to 6 membered heteroaryl.

14. The compound of one of claims 1 to 11, wherein R¹ is substituted or unsubstituted phenyl.

15. The compound of one of claims 1 to 11, wherein R¹ is R²-substituted or unsubstituted phenyl or R²-substituted or unsubstituted 5 to 6 membered heteroaryl;
R² is independently halogen, —CX²₃, —CHX²₂, —CH₂X², —OCX²₃, —OCH₂X², —OCHX²₂, —CN, —SOₙ₂R^{2A}, —SOᵥ₂NR^{2A}R^{2B}, —NR^{2C}C(O)NR^{2A}R^{2B}, —N(O)ₘ₂, —NR^{2A}R^{2B}, NR^{2C}NR^{2A}R^{2B}, —C(O)R^{2A}, —C(O)OR^{2A}, —C(O)NR^{2A}R^{2B}, —C(O)NR^{2C}NR^{2A}R^{2B}, —OR^{2A}, —NR^{2A}SO₂R^{2B}, —NR^{2A}C(O)R^{2B}, —NR^{2A}C(O)OR^{2B}, —NR^{2A}OR^{2B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, and R^{2C} are independently hydrogen, —CX₃, —CHX₂, —CH₂X, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
m2 is independently 1 or 2;
v2 is independently 1 or 2;
n2 is independently an integer from 0 to 4; and
X and X² are independently —Cl, —Br, —I or —F.

16. The compound of one of claims 1 to 15, having the formula: 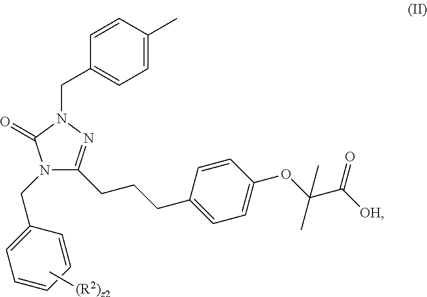
wherein z2 is an integer from 0 to 5.

17. The compound of claim 16, wherein z2 is 1.

18. The compound of claim 16, wherein z2 is 2.

19. The compound of claim 16, wherein z2 is 3.

20. The compound of one of claims 1 to 15, having the formula:

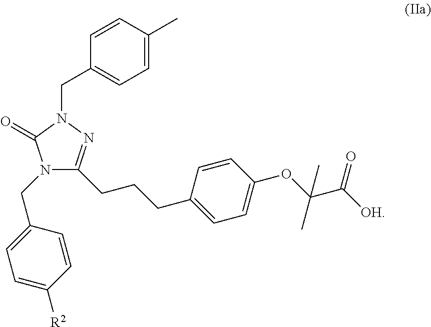

21. The compound of one of claims 1 to 15, having the formula:

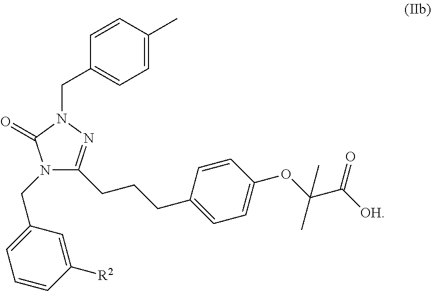

22. The compound of one of claims 1 to 15, having the formula:

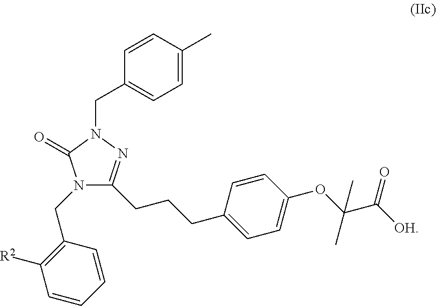

23. The compound of one of claims 1 to 15, having the formula:

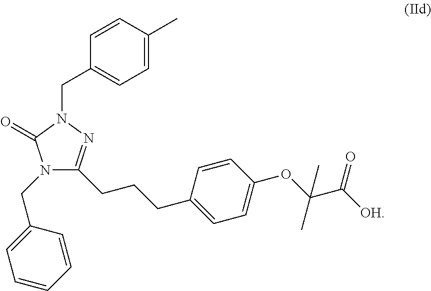 [SMILES: Cc1ccc(Cn2nc(CCCc3ccc(OC(C)(C)C(=O)O)cc3)n(Cc3ccccc3)c2=O)cc1]

24. The compound of one of claims 1 to 22, wherein R² is independently halogen, —CX²₃, —CHX²₂, —CH₂X², —OCX²₃, —OCH₂X², —OCHX²₂, —OH, substituted or unsubstituted C₁-C₄ alkyl, or substituted or unsubstituted 2 to 4 membered heteroalkyl; and
X² is independently —Cl, —Br, —I or —F.

25. The compound of one of claims 1 to 22, wherein R² is independently halogen, —OCH₃, —OH, or unsubstituted C₁-C₄ alkyl.

26. The compound of claim 1, having the formula:

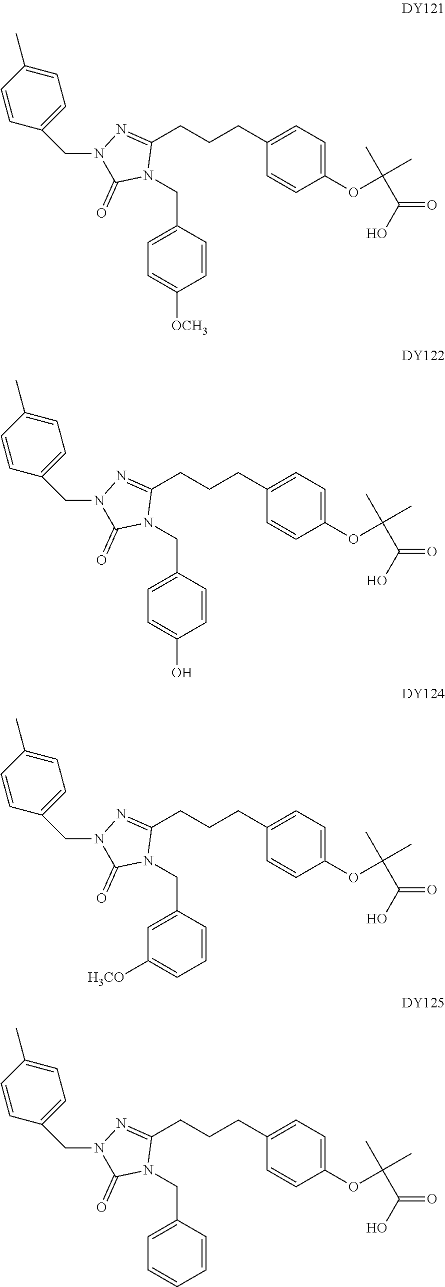 [SMILES: COc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(O)cc3)c2=O)cc1 ; COc1cccc(Cn2c(CCCc3ccc(OC(C)(C)C(=O)O)cc3)nn(Cc3ccc(C)cc3)c2=O)c1 ; Cc1ccc(Cn2nc(CCCc3ccc(OC(C)(C)C(=O)O)cc3)n(Cc3ccccc3)c2=O)cc1]

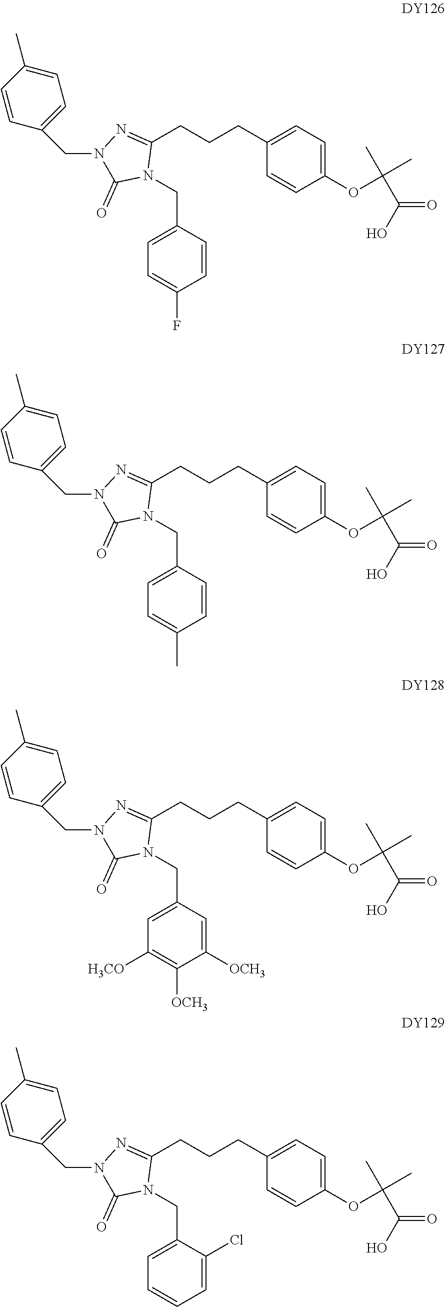 [SMILES: Cc1ccc(Cn2nc(CCCc3ccc(OC(C)(C)C(=O)O)cc3)n(Cc3ccc(F)cc3)c2=O)cc1 ; Cc1ccc(Cn2nc(CCCc3ccc(OC(C)(C)C(=O)O)cc3)n(Cc3ccc(C)cc3)c2=O)cc1 ; COc1cc(Cn2c(CCCc3ccc(OC(C)(C)C(=O)O)cc3)nn(Cc3ccc(C)cc3)c2=O)cc(OC)c1OC ; Cc1ccc(Cn2nc(CCCc3ccc(OC(C)(C)C(=O)O)cc3)n(Cc3ccccc3Cl)c2=O)cc1]

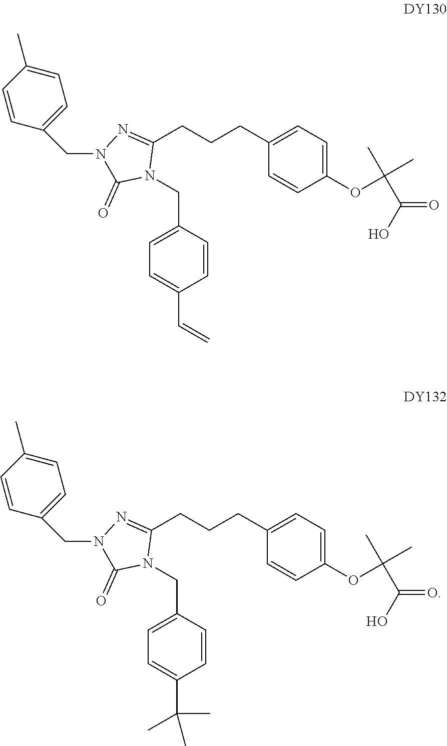 [SMILES: C=Cc1ccc(Cn2c(CCCc3ccc(OC(C)(C)C(=O)O)cc3)nn(Cc3ccc(C)cc3)c2=O)cc1 ; Cc1ccc(Cn2nc(CCCc3ccc(OC(C)(C)C(=O)O)cc3)n(Cc3ccc(C(C)(C)C)cc3)c2=O)cc1]

27. The compound of claim 1, having the formula:

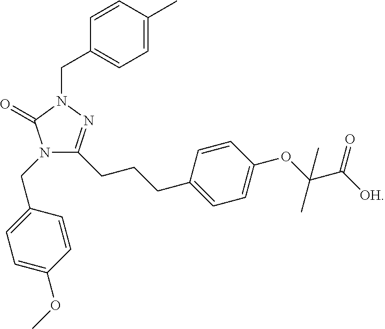 [SMILES: COc1ccc(Cn2c(CCCc3ccc(OC(C)(C)C(=O)O)cc3)nn(Cc3ccc(C)cc3)c2=O)cc1]

28. A pharmaceutical composition comprising the compound of any one of claims 1 to 27 and a pharmaceutically acceptable excipient.

29. A method of increasing a peroxisome proliferator-activated receptor activity, said method comprising contacting the peroxisome proliferator-activated receptor with the compound of one of claims 1 to 27.

30. The method of claim 29, wherein the peroxisome proliferator-activated receptor is peroxisome proliferator-activated receptor α.

31. A method of treating non-alcoholic fatty liver disease in a subject in need thereof, the method comprising administering to the subject a compound of one of claims 1 to 27.

32. A method of treating non-alcoholic steatohepatitis in a subject in need thereof, the method comprising administering to the subject a compound of one of claims 1 to 27.

33. A method of treating obesity in a subject in need thereof, the method comprising administering to the subject a compound of one of claims 1 to 27.

34. A method of treating diabetes in a subject in need thereof, the method comprising administering to the subject a compound of one of claims 1 to 27.

35. A method of treating metabolic syndrome in a subject in need thereof, the method comprising administering to the subject a compound of one of claims 1 to 27.

36. A method of treating a lipid disorder in a subject in need thereof, the method comprising administering to the subject a compound of one of claims 1 to 27.

37. The method of claim 36, wherein the lipid disorder is dyslipidemia, hyperlipidemia, hypertriglyceridemia, hypercholesterolemia, hyperlipoproteinemia, combined hyperlipidemia, hyperchylomicronemia, familial hyperchylomicronemia, familial apoprotein CII deficiency, familial hypercholesterolemia, familial combined hyperlipidemia, familial dysbetalipoproteinemia, or familial hypertriglyceridemia.

38. A method of modulating the level of a lipid in a subject in need thereof, the method comprising administering to the subject a compound of one of claims 1 to 27.

39. The method of claim 38, wherein the method modulates the level of chylomicrons, triglycerides, phospholipids, cholesterol, lipoproteins, very-low-density lipoproteins, intermediate-density lipoproteins, low-density lipoproteins, or high-density lipoproteins.

40. The method of claim 38, wherein the method decreases the level of chylomicrons, triglycerides, phospholipids, cholesterol, lipoproteins, very-low-density lipoproteins, intermediate-density lipoproteins, or low-density lipoproteins.

41. The method of claim 38, wherein the method increases the level of high-density lipoproteins.


SEQUENCE LISTING (4 sequences)

SEQ ID NO: 1 - DNA, 36 bp, Artificial sequence
  Synthetic polynucleotide
gcgggatccgtggacacggaaagcccactctgcccc

SEQ ID NO: 2 - DNA, 32 bp, Artificial sequence
  Synthetic polynucleotide
atctcagtacatgtccctgtagatctcctgca

SEQ ID NO: 3 - DNA, 22 bp, Artificial sequence
  Synthetic polynucleotide
atcatggacaccaaacatttcc

SEQ ID NO: 4 - DNA, 18 bp, Artificial sequence
  Synthetic polynucleotide
ctaagtcatttggtgcgg